(19) United States
(12) Patent Application Publication
(10) Pub. No.: US 2020/0263167 A1
(43) Pub. Date: Aug. 20, 2020

(54) DEVICES, SYSTEMS AND METHODS FOR BIOMARKER ANALYSIS

(71) Applicant: JUNO DIAGNOSTICS, INC., San Diego, CA (US)

(72) Inventors: Dirk VAN DEN BOOM, Encinitas, CA (US); Mathias EHRICH, San Diego, CA (US); Paul OETH, San Diego, CA (US); Jim CHAUVAPUN, San Diego, CA (US)

(21) Appl. No.: 16/648,819

(22) PCT Filed: Sep. 26, 2018
(86) PCT No.: PCT/US2018/052891

§ 371 Date: Mar. 19, 2020

Related U.S. Application Data
(60) Provisional application No. 62/563,314, filed on Sep. 26, 2017.
(60) Provisional application No. 62/609,086, filed on Dec. 21, 2017.

(51) Int. Cl. C12N 15/10; C12Q 1/686

(52) U.S. Cl. CPC C12N 15/1017; C12Q 2565/625; C12Q 1/686

(57) ABSTRACT

Provided herein are devices, systems, kits and methods for predicting or determining the gender of a fetus using cell free fetal nucleic acids in a small amount of maternal biological sample. Devices can be used at point of need during early stages of pregnancy and are compatible with communication devices.

### RELATED APPLICATIONS

This application claims priority to U.S. Provisional Patent Application No. 62/563,314 filed on Sep. 26, 2017 and U.S. Provisional Patent Application No. 62/609,086 filed on Dec. 21, 2017. Priority is claimed pursuant to 35 U.S.C. § 119. The above noted patent applications are incorporated by reference as if set forth fully herein.

### BACKGROUND OF THE INVENTION

Genetic testing is a means for obtaining information about a subject's DNA and/or expression of that DNA. Genetic tests are continually being developed to obtain biological information about a subject. This biological information has many uses, including determining a health status of an individual, diagnosing an individual with an infection or disease, determining a suitable treatment for the individual, solving a crime and identifying paternity. Currently, genetic testing is mainly performed in clinics and laboratories by trained personnel with expensive and bulky equipment that requires technical training and expertise to use. It typically takes days to weeks, from the time a biological sample is obtained from a patient, to provide the patient with results of a genetic test.

As an example, many people who become aware of a pregnancy are eager to know the sex (hereby referred to as gender throughout this application) of the baby as soon as possible. There are tests that allow for obtaining gender information from DNA in maternal blood. Blood obtained from the mother must be analyzed with sophisticated equipment by a highly-trained technician. If the blood is obtained at a site distant from the laboratory where DNA analysis is performed, the sample must be stored, shipped, and analyzed in a timely fashion, or otherwise risk sample degradation.

### SUMMARY OF THE INVENTION

Disclosed herein are devices, systems, kits and methods for analyzing components (e.g., nucleic acids, proteins) of a biological sample, including a sample from an animal (human or non-human), an environment (e.g., water, soil), a plant, bacteria, and food. In general, devices, systems, kits and methods disclosed herein are capable of providing genetic information from a very low volume of a sample by taking advantage of cell-free DNA fragmentation. Cell-free DNA fragmentation creates statistically independent markers from repetitive regions (e.g., regions with a common sequence) and/or multiple detection regions along a target region. By way of non-limiting example, cell-free DNA fragments from repetitive regions (e.g., regions of the genome containing multiple copies of the same or similar sequence) are present at a higher effective concentration in a sample than DNA fragments having sequences that are not present in multiple copies. Advantageously, fragments from repetitive regions may be amplified with a single pair of primers or detected with a single probe. However, multiple detection regions do not have to share similar sequences. Such fragments may also be detected in small volumes, e.g., by tagging and amplifying them with a universal primer or amplifying with multiple primer pairs (e.g. in a multiplexed format).

Analysis of cell-free circulating nucleic acids is met with a number of technical challenges. For instance, amplification of circulating nucleic acids in blood may be inhibited by some of the components in whole blood (e.g., hemoglobin and associated iron). The devices, systems, kits and methods disclosed herein aim to overcome many of these technical challenges. In addition, the devices, systems, kits and methods offer the advantage of being (1) minimally invasive, (2) applicable in home with little or no technical training (e.g., do not require complex equipment); and (3) informative at early stages of a condition (e.g., pregnancy, infection). Furthermore, the avoidance of repeated doctor/hospital visits for the purpose of blood draws and centralized testing may improve patient compliance and allow more frequent monitoring, ultimately leading to improved health outcomes at lower cost to the healthcare system.

Disclosed herein, in some aspects, are devices that comprise a sample purifier for removing a cell from a biological fluid sample to produce a cell-depleted sample; and at least one of a detection reagent and a signal detector for detecting a plurality of biomarkers in the cell-depleted sample. In some instances, the plurality of biomarkers comprises multiple cell-free DNA fragments. In some instances, each of the multiple cell-free fragments comprises a region represented by a first sequence or a second sequence at least 90% homologous to the first sequence. In some instances, the first sequence is physically distant enough from the second sequence such that the first sequence is present on a first cell-free nucleic acid of the subject and the second sequence is present on a second cell-free nucleic acid of the subject. In some instances, the device comprises at least one nucleic acid amplification reagent and a single pair of primers capable of amplifying the first sequence and the second sequence. In some instances, at least one of the first sequence and the second sequence is repeated at least two times in the genome of the subject. In some instances, at least one of the first sequence and the second sequence is repeated at least three times in the genome of the subject. In some instances, at least one of the first sequence and the second sequence is repeated at least four times in the genome of the subject. In some instances, at least one of the first sequence and the second sequence is repeated at least five times in the genome of the subject. In some instances, the first sequence and the second sequence are each at least 10 nucleotides in length. In some instances, the first sequence is on a first chromosome and the second sequence is on a second chromosome. In some instances, the first sequence and the second sequence are on the same chromosome but separated by at least 1 nucleotide. In some instances, the first sequence and the second sequence are in functional linkage. In some instances, the first sequence is at least 80% identical to the second sequence. In some instances, the sample purifier comprises a filter. In some instances, the sample purifier comprises a wicking material or capillary device for pushing the biological fluid through the filter. In some instances, the filter has a pore size of about 0.05 microns to about 2 microns. In some instances, the sample purifier comprises a binding moiety that binds a nucleic acid, protein, cell surface marker, or microvesicle surface marker in the fluid sample. In some instances, the binding moiety comprises an antibody, antigen binding antibody fragment, a ligand, a receptor, a peptide, a small molecule, or a combination thereof. In some instances, the binding moiety is capable of binding an extracellular vesicle, wherein the extracellular vesicle is released from a fetal cell or a placental cell of the female subject. In some instances, the at least one nucleic acid amplification reagent comprises at least one isothermal amplification reagent. In some instances, the at least one isothermal amplification reagent comprises a recombinase polymerase, a single-strand DNA-binding protein, a strand-displacing polymerase, or a combination thereof. In some instances, the signal detector comprises a solid support. In some instances, the solid support is a column. In some instances, the solid support comprises a binding moiety that binds the amplification product. In some instances, the binding moiety is an oligonucleotide. In some instances, the signal detector is a lateral flow strip. In some instances, devices comprise a detection reagent, wherein the detection reagent comprises a gold particle or a fluorescent particle. In some instances, the sample purifier removes cells from blood, and the cell-depleted sample is plasma. In some instances, the device is contained in a single housing. In some instances, the device operates at room temperature. In some instances, the device is capable of detecting the plurality of biomarkers in the cell-depleted sample within about five minutes to about twenty minutes of receiving the biological fluid. In some instances, devices comprise a transport or storage compartment. In some instances, the transport or storage compartment comprises an absorption pad or a fluid container. In some instances, devices comprise a communication connection. In some instances, the communication connection is a wireless communication system, a cable, or a cable port. In some instances, devices comprise a transdermal puncture device.

Further disclosed herein, in some aspects, are methods that comprise obtaining a fluid sample from a subject, wherein the volume of the biological sample is not greater than about 300 μL; contacting at least one cell free nucleic acid in the fluid sample with an amplification reagent and an oligonucleotide primer that anneals to a sequence corresponding to a sequence of interest; and detecting the presence or absence of an amplification product, wherein the presence or absence indicates a health status of the subject. In some instances, the fluid sample is a blood sample. In some instances, the volume of the blood sample is not greater than 120 μl. In some instances, the fluid sample is a plasma sample from blood. In some instances, the volume of the plasma sample is not greater than 50 μl. In some instances, the volume of the plasma sample is between about 5 μl and about 40 μl. In some instances, the volume of the plasma sample is between about 10 μl and about 40 μl. In some instances, obtaining comprises performing a finger prick. In some instances, methods comprise milking a pricked finger to increase blood that comes from the finger prick. In some instances, obtaining the blood sample does not comprise performing a phlebotomy. In some instances, the fluid sample is a urine sample. In some instances, the fluid sample comprises a lachrymal secretion (a tear). In some instances, the fluid sample comprises interstitial fluid. In some instances, the fluid sample comprises saliva. In some instances, methods comprise removing at least one of a cell, a cell fragment, and a microparticle, from the fluid sample. In some instances, the sample contains about 25 pg to about 250 pg of total circulating cell free DNA. In some instances, the sample comprises cell free DNA fragments having a length of about 20 base pairs to about 160 base pairs in length. In some instances, the sample comprises cell free DNA fragments having a length of about 20 base pairs to about 250 base pairs in length. In some instances, the sample contains about 5 to about 100 copies of a sequence of interest. In some instances, the sequence of interest is at least 10 nucleotides in length. In some instances, the copies are at least 90% identical to one another. In some instances, amplifying comprises isothermal amplification. In some instances, amplifying occurs at room temperature. In some instances, the method comprises incorporating a tag into the amplification product as the amplifying occurs, and wherein detecting the at least one amplification product comprises detecting the tag. In some instances, the tag does not comprise a nucleotide. In some instances, detecting the amplification product comprises contacting the amplification product with a binding moiety that is capable of interacting with the tag. In some instances, methods comprise contacting the amplification product with the binding moiety on a lateral flow device. In some instances, methods are performed in less than fifteen minutes. In some instances, methods are performed in less than thirty minutes. In some instances, methods are performed in less than sixty minutes. In some instances, methods are performed by the subject. In some instances, methods are performed by an individual without receiving technical training for performing the method. In some instances, methods comprise obtaining, contacting, and detecting with a single handheld device. In some instances, the subject performs the obtaining by pressing their skin against a transdermal puncture device of the handheld device. In some instances, the subject presses their skin against the transdermal puncture device not more than once. In some instances, the subject presses their skin against the transdermal puncture device not more than twice. In some instances, the health status is selected from the presence and the absence of a pregnancy. In some instances, the health status is a presence of a neurological disorder. In some instances, the health status is an absence of a neurological disorder. In some instances, the health status is a presence of a metabolic disorder. In some instances, the health status is an absence of a metabolic disorder. In some instances, the health status is a presence of a cancer. In some instances, the health status is an absence of a cancer. In some instances, the health status is a presence of an autoimmune disorder. In some instances, the health status is an absence of an autoimmune disorder. In some instances, the health status is a presence of an allergic reaction. In some instances, the health status is an absence of an allergic reaction. In some instances, the health status is a presence of an infection. In some instances, the health status is an absence of an infection. In some instances, the health status is a presence of an inherited genetic or epigenetic disease. In some instances, the health status is an absence of an inherited genetic or epigenetic disease. In some instances, the health status is a response to a drug or a therapy.

By way of non-limiting example, devices, systems, kits and methods are disclosed herein may be used for determining the gender of a fetus. Devices, systems, kits and methods disclosed herein allow for gender determination in the privacy of a home, without the need for laboratory equipment and without the risk of sample swapping. These devices, systems, kits and methods generally analyze cell free fetal DNA and/or cell free fetal RNA. Devices, systems, kits and methods disclosed herein may advantageously determine the gender of the fetus at early stages of gestation because they require very little fetal nucleic acid material. Devices, systems, kits and methods disclosed herein provide gender status from a very low volume of sample because the devices, systems, kits and methods are capable of detecting fragments of the Y chromosome including genes or any amplifiable regions which can uniquely identify the presence or absence of the Y chromosome in a biological sample, often present in multiple copies on the Y chromosome. The effective concentration of these fragments is higher than those fragments of genes that are not present in multiple copies in most cases.

In some aspects, disclosed herein are devices comprising: a sample purifier that removes a cell from a fluid sample of a female subject; at least one nucleic acid amplification reagent; at least one oligonucleotide comprising a sequence corresponding to a Y chromosome, wherein the at least one oligonucleotide and nucleic acid amplification reagent are capable of producing an amplification product; and at least one of a detection reagent or a signal detector for detecting the amplification product. In some instances, the fluid sample is blood. In some instances, the sample purifier comprises a filter. In some instances, the sample purifier comprises a wicking material or capillary device for pushing the biological fluid through the filter. In some instances, the filter has a pore size of about 0.05 microns to about 2 microns. In some instances, the sample purifier comprises a binding moiety that binds a nucleic acid, protein, cell surface marker, or microvesicle surface marker in the biological sample. In some instances, the binding moiety comprises an antibody, antigen binding antibody fragment, a ligand, a receptor, a peptide, a small molecule, or a combination thereof. In some instances, the binding moiety is capable of binding an extracellular vesicle, wherein the extracellular vesicle is released from a fetal cell or a placental cell of the female subject. In some instances, the binding moiety binds a human chorionic gonadotropin protein or a transcript of a human chorionic gonadotropin encoding gene. In some instances, the at least one oligonucleotide comprises a primer that hybridizes to a Y chromosome sequence. In some instances, the at least one oligonucleotide comprises a probe that hybridizes to a Y chromosome sequence, and wherein the probe comprises an oligonucleotide tag. In some instances, the oligonucleotide tag is not specific to a Y chromosome sequence. In some instances, the device comprises at least one primer that hybridizes to the oligonucleotide tag, and produces an amplification product in the presence of the amplification reagent. In some instances, the at least one nucleic acid amplification reagent comprises at least one isothermal amplification reagent. In some instances, the at least one isothermal amplification reagent comprises a recombinase polymerase, a single-strand DNA-binding protein, a strand-displacing polymerase, or a combination thereof. In some instances, the signal detector comprises a solid support. In some instances, the solid support is a bead. In some instances, the solid support comprises a binding moiety that binds the amplification product. In some instances, the binding moiety is an oligonucleotide. In some instances, the signal detector is a lateral flow strip. In some instances, the detection reagent comprises a gold particle. In some instances, the detection reagent comprises a fluorescent particle. In some instances, the device is contained in a single housing. In some instances, the device operates at room temperature. In some instances, the device detects the amplification product within about five minutes to about twenty minutes of receiving the biological fluid. In some instances, the device comprises a transport or storage compartment. In some instances, the transport or storage compartment comprises an absorption pad or a fluid container. In some instances, the device comprises a communication connection. In some instances, the communication connection is a wireless communication system, a cable, or a cable port. In some instances, the device comprises a transdermal puncture device.

In some aspects, disclosed herein are kits that comprise a device disclosed herein, and a transdermal puncture device. In some instances, the transdermal puncture device is a lancet. In some instances, the device comprises a capillary for drawing up blood from a transdermal puncture. In some instances, the kit comprises a container, pouch, wire or cable for heating or cooling the device of a component thereof.

In some aspects, disclosed herein are methods comprising: obtaining a fluid sample from a female pregnant subject, wherein the volume of the biological sample is not greater than about 300 μL; contacting at least one cell free nucleic acid in the fluid sample with an amplification reagent and an oligonucleotide primer that anneals to a sequence corresponding to a sex chromosome; and detecting the presence or absence of an amplification product, wherein the presence or absence indicates the gender of a fetus of the female pregnant subject. In some instances, the fluid sample is a blood sample. In some instances, obtaining comprises performing a finger prick. In some instances, obtaining comprises milking a pricked finger to increase blood that comes from the finger prick. In some instances, obtaining the blood sample does not comprise performing a phlebotomy. In some instances, the fluid sample is a urine sample. In some instances, the fluid sample is a saliva sample. In some instances, methods comprise removing at least one of a cell, a cell fragment, and a microparticle, from the fluid sample. In some instances, the sample contains about 25 pg to about 250 pg of total circulating cell free DNA. In some instances, the cell free nucleic acid comprises a cell free fetal DNA fragment. In some instances, the cell free fetal DNA fragment is about 20 base pairs to about 160 base pairs in length. In some instances, the sequence corresponding to the sex chromosome is a Y chromosome sequence that is present in at least two copies on the Y chromosome. In some instances, Y chromosome sequence is a sequence present in a DYS14 gene or a TTTY22 gene. In some instances, the sample does not contain more than about 100 copies of the cell free nucleic acid. In some instances, the sample contains about 5 to about 100 copies of the cell free nucleic acid. In some instances, the female pregnant subject is not more than 8 weeks pregnant. In some instances, amplifying comprises isothermal amplification. In some instances, amplifying occurs at room temperature. In some instances, amplifying comprises contacting the circulating cell free nucleic acid with a recombinase polymerase. In some instances, methods comprise tagging the cell free nucleic acid with an oligonucleotide tag. In some instances, amplifying comprises contacting the cell free nucleic acid with at least one oligonucleotide primer having a sequence corresponding to the oligonucleotide tag. In some instances, the oligonucleotide primer comprises a blocking group that prevents extension of the oligonucleotide primer until at least one of an amplification condition and amplification reagents are provided. In some instances, methods comprises incorporating a tag into the amplification product as the amplifying occurs, and wherein detecting the at least one amplification product comprises detecting the tag. In some instances, detecting the amplification product comprises detecting an amplified oligonucleotide tag. In some instances, the tag comprises a nucleotide. In some instances, the tag does not comprise a nucleotide. In some instances, detecting the amplification product comprises contacting the amplification product with a binding moiety that is capable of interacting with the tag or oligonucleotide tag. In some instances, methods comprise contacting the amplification product with the binding moiety on a lateral flow device. In some instances, steps (a) through (c) are performed in less than fifteen minutes. In some instances, the method is performed by the subject. In some instances, methods are performed by an individual without receiving technical training to perform the method. In some instances, the volume is not greater than 120 μL.

In some aspects, disclosed herein are methods comprising obtaining a fluid sample from a female pregnant subject with a handheld device, wherein the volume of the fluid sample is not greater than about 300 μL; sequencing at least one cell free nucleic acid in the fluid sample with the handheld device; detecting the presence or absence of a sequence corresponding to a Y chromosome through a display in the handheld device, thereby determining a gender of a fetus in the female pregnant subject; and communicating, with the handheld device, the gender to another subject. In some instances, detecting and communicating occur simultaneously. In some instances, the volume is not greater than 120 μL. In some instances, obtaining does not comprise a phlebotomy. In some instances, the female pregnant subject performs the obtaining by pressing her skin against a transdermal puncture device of the handheld device. In some instances, the female pregnant subject presses a finger against the transdermal puncture device. In some instances, the female pregnant subject presses her skin against the transdermal puncture device not more than once. In some instances, the female pregnant subject presses her skin against the transdermal puncture device not more than twice.

In some aspects, disclosed herein are devices that comprise a sample purifier for removing a cell from a biological fluid sample to produce a cell-depleted sample; and at least one of a detection reagent and a signal detector for detecting a plurality of cell-free DNA fragments in the cell-depleted sample. In some instances, a first sequence is present on a first cell-free DNA fragment of the plurality of cell-free DNA fragments and a second sequence is present on a second cell-free DNA fragment of the plurality of cell-free DNA fragments, and wherein the first sequence is at least 80% identical to the second sequence. In some instances, the device comprises at least one nucleic acid amplification reagent and a single pair of primers capable of amplifying the first sequence and the second sequence. In some instances, at least one of the first sequence and the second sequence is repeated at least twice in a genome of a subject. In some instances, the first sequence and the second sequence are each at least 10 nucleotides in length. In some instances, the first sequence is on a first chromosome and the second sequence is on a second chromosome. In some instances, the first sequence and the second sequence are on the same chromosome but separated by at least 1 nucleotide. In some instances, the first sequence and the second sequence are in functional linkage. In some instances, the sample purifier comprises a filter, and wherein the filter has a pore size of about 0.05 microns to about 2 microns. In some instances, the filter is a vertical filter. In some instances, the sample purifier comprises a binding moiety selected from an antibody, antigen binding antibody fragment, a ligand, a receptor, a peptide, a small molecule, and a combination thereof. In some instances, the binding moiety is capable of binding an extracellular vesicle. In some instances, the at least one nucleic acid amplification reagent comprises an isothermal amplification reagent. In some instances, the signal detector is a lateral flow strip. In some instances, the device is contained in a single housing. In some instances, the device operates at room temperature. In some instances, the device is capable of detecting the plurality of biomarkers in the cell-depleted sample within about five minutes to about twenty minutes of receiving the biological fluid. In some instances, the device comprises a communication connection. In some instances, the device comprises a transdermal puncture device.

Further disclosed herein, in some aspects, are methods that comprise obtaining a fluid sample from a subject, wherein the volume of the biological sample is not greater than about 120 microliters; contacting at least one cell free nucleic acid in the fluid sample with an amplification reagent and an oligonucleotide primer that anneals to a sequence corresponding to a sequence of interest in order to produce an amplification product; and detecting the presence or absence of the amplification product, wherein the presence or absence indicates a health status of the subject. In some instances, the fluid sample is a blood sample. In some instances, the fluid sample is a plasma sample from blood. In some instances, the volume of the plasma sample is not greater than 50 In some instances, the volume of the plasma sample is between about 10 μl and about 40 μl. In some instances, the sample contains about 25 pg to about 250 pg of total circulating cell free DNA. In some instances, the sample contains about 5 to about 100 copies of the sequence of interest. In some instances, the copies are at least 90% identical to one another. In some instances, the sequence of interest is at least 10 nucleotides in length. In some instances, contacting comprises performing isothermal amplification. In some instances, contacting occurs at room temperature. In some instances, the method comprises incorporating a tag into the amplification product as the amplifying occurs, and wherein detecting the presence of the amplification product comprises detecting the tag. In some instances, the tag does not comprise a nucleotide. In some instances, detecting the amplification product comprises contacting the amplification product with a binding moiety that is capable of interacting with the tag. In some instances, methods comprise contacting the amplification product with the binding moiety on a lateral flow device. In some instances, steps (a) through (c) are performed in less than fifteen minutes. In some instances, the method is performed by the subject. In some instances, the method is performed by an individual without receiving technical training for performing the method. In some instances, obtaining, contacting, and detecting is performed with a single handheld device. In some instances, the health status is selected from the presence and the absence of a pregnancy. In some instances, the health status is selected from the presence and the absence of a neurological disorder, a metabolic disorder, a cancer, an autoimmune disorder, an allergic reaction, and an infection. In some instances, the health status is a response to a drug or a therapy.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the methods, devices, systems and kits disclosed herein are set forth with particularity in the appended claims. A better understanding of the features and advantages of the methods, devices, systems and kits disclosed herein will be obtained by reference to the following detailed description that sets forth illustrative embodiments, in which the principles of the methods, devices, systems and kits disclosed herein are utilized, and the accompanying drawings of which:

FIG. 1 shows success of amplification and detection of both single target sequences and multi-copy target sequences in the form of (unfragmented) genomic DNA and cell-free DNA.

FIG. 2 shows an exemplary workflow for methods using devices, systems and kits disclosed herein.

FIG. 3 shows amplification of DNA from a Y chromosome using 80 μl of whole blood applied to a device disclosed herein.

FIG. 4 shows a Y chromosome DNA amplified by recombinase polymerase amplification on a polyacrylamide gel. Specific primer sequences and amplicon sequences are listed in Tables 3 and 4. Expected sizes, amplicons and primers are as follows: Lane 1: LMW Ladder, Lane 2: NTC TwistDx, Lane 3: PosCon TwistDx (143 bp); Lane 4: SRY 6—NTC, primers not shown; Lane 5: SRY 6—Male (370 bp), primers not shown; Lane 6: DYS14—Amp10 (134 bp), primers DYS14_1_F_Long and DYS14_5_R_Long: Lane 7: DYS14—Amp 11 (148 bp), primers DYS14_5_F_long and DYS14_4_R_long; Lane 8: TTTY22—Amp 10 (118 bp), primers TTTY22_1_F_long and TTTY22_2_R_long; Lane 9: TTTY22—Amp 11 (121 bp), primers TTTY22_7_F_long and TTTY22_6_R_long; Lane 10: TTTY22—Amp 12 (123 bp), primers TTTY22_6_F-long and TTTY22_4_R_long; Lane 11: SRY—Amp 10 (178 bp), primers not shown; Lane 12: SRY—Amp 11 (213 bp), primers not shown; Lane 13: SRY—Amp 12 (161 bp), primers not shown; Lane 14: LMW Ladder.

FIG. 5 shows real-time detection of DYS14 Y-chromosome amplification products from recombinase polymerase amplification.

FIG. 6 shows real-time detection of DYS14 Y-chromosome amplification products from recombinase polymerase amplification with female control samples.

FIG. 7 shows lateral flow detection of Y-chromosome DYS14 recombinase polymerase amplification products

FIG. 8 shows examples of how a mobile device may be used to display, interpret, and/or share results obtained from devices and methods disclosed herein. FIG. 8A shows an overview of the functionality of a mobile application that can be used in connection with devices, systems and kits disclosed herein. FIG. 8B shows a non-limiting example of a graphic user interface for a mobile application; in this case, an interface providing a step-by-step walkthrough to guide a user through use of the devices, systems and kits disclosed herein. FIG. 8C shows a non-limiting example of a graphic user interface for a mobile application; in this case, an interface providing a home screen allowing a user to access the mobile application functionality disclosed herein. FIG. 8D shows a non-limiting example of a graphic user interface for a mobile application; in this case, an interface providing a progress diagram informing a user of the status of a process for connecting to a device, system, or kit disclosed herein to receive information. FIG. 8E shows a non-limiting example of a graphic user interface for a mobile application; in this case, an interface providing a gender test report to a user. FIG. 8F shows a non-limiting example of a graphic user interface for a mobile application; in this case, an interface providing a social sharing screen allowing a user to access features to share gender test results. FIG. 8G shows a non-limiting example of a graphic user interface for a mobile application; in this case, an interface providing a home screen allowing a user to access additional features such as a pregnancy blog and timeline of important pregnancy-related events.

FIG. 9 shows an agarose gel with RPA products generated for the TSPY1 (DYS14) loci on the Y chromosome.

FIG. 10 shows nucleic acid lateral flow immunoassay strips with human TSPY1 (DYS14) Y chromosome RPA-LF products.

FIG. 11 shows amplicons from a highly repetitive Y-chromosome region (HRYR) are generated with a sample from a male human.

FIG. 12 shows amplicons from a highly repetitive Y-chromosome region (HRYR) are not generated with a sample from a female human.

FIG. 13 shows a comparison between plasma separated from less than 50 microliters (μ1) of male whole blood using the Vivid™ Membrane vs. standard centrifugation methodology.

FIG. 14 shows yields from bead based and column based purification of 20 μl of human plasma as input for extraction from male and female subjects.

FIG. 15A-C show an exemplary device disclosed herein. FIG. 15A shows a side view of the exemplary device. FIG. 15B shows a top view of the exemplary device. FIG. 15C shows a front view of the exemplary device.

### CERTAIN TERMINOLOGIES

The following descriptions are provided to aid the understanding of the methods, systems and kits disclosed herein. The following descriptions of terms used herein are not intended to be limiting definitions of these terms. These terms are further described and exemplified throughout the present application.

In general, the terms “cell free polynucleotide,” and “cell free nucleic acid,” used interchangeably herein, refer to polynucleotides or nucleic acids that can be isolated from a sample without extracting the polynucleotide or nucleic acid from a cell. A cell-free nucleic acid is a nucleic acid that is not contained within a cell membrane, i.e., it is not encapsulated in a cellular compartment. In some embodiments, a cell-free nucleic acid is a nucleic acid that is not bounded by a cell membrane and is circulating or present in blood or other fluid. In some embodiments, the cell-free nucleic acid is cell-free before and/or upon collection of the biological sample containing it, and is not released from the cell as a result of sample manipulation by man, intentional or otherwise, including manipulation upon or after collection of the sample. In some instances, cell-free nucleic acids are produced in a cell and released from the cell by physiological means, including, e.g., apoptosis, and non-apoptotic cell death, necrosis, autophagy, spontaneous release (e.g., of a DNA/RNA-lipoprotein complex), secretion, and/or mitotic catastrophe. In some embodiments, a cell-free nucleic acid comprises a nucleic acid that is released from a cell by a biological mechanism, (e.g., apoptosis, cell secretion, vesicular release). In further or additional embodiments, a cell-free nucleic acid is not a nucleic acid that has been extracted from a cell by human manipulation of the cell or sample processing (e.g., cell membrane disruption, lysis, vortex, shearing, etc.).

In some instances, the cell-free nucleic acid is a cell-free fetal nucleic acid. In general, the term, “cell free fetal nucleic acid,” as used herein, refers to a cell-free nucleic acid, as described herein, wherein the cell-free nucleic acid is from a cell that comprises fetal DNA. Often, a large portion of cell-free fetal nucleic acids are found in maternal biological samples as a result of placental tissue being regularly shed during the pregnant subject's pregnancy. Often, many of the cells in the placental tissue shed are cells that contain fetal DNA. Thus, in some instances, a cell-free fetal nucleic acid is a nucleic acid released from a placental cell.

In some instances, cellular nucleic acids (nucleic acids contained by cells) are intentionally or unintentionally released from cells by devices and methods disclosed herein. However, these are not considered “cell-free nucleic acids,” as the term is used herein. In some instances, devices, systems, kits and methods disclosed herein provide for analyzing cell-free nucleic acids in biological samples, and in the process analyze cellular nucleic acids as well.

As used herein, the term “cellular nucleic acid” refers to a polynucleotide that is contained in a cell. A cellular nucleic acid may be described as a nucleic acid that can be released from a cell due to manipulation of the biological sample. Non-limiting examples of manipulation of the biological sample include centrifuging, vortexing, shearing, mixing, lysing, and adding a reagent (e.g., detergent, buffer, salt, enzyme) to the biological sample that is not present in the biological sample when it is obtained. A cellular nucleic acid may be described as a nucleic acid that can be released from a cell due to lysis conditions (e.g., shearing, lysis buffers). A cellular nucleic acid may be described as a nucleic acid that can be released from a cell due to contacting the biological sample with a lysis reagent. Exemplary lysis reagents are disclosed herein. In some instances, the cellular nucleic acid is a nucleic acid that has been released from a cell due to disruption or lysis of the cell by a machine, human or robot.

As used herein, the term “biomarker” generally refers to any marker of a subject's biology or condition. A biomarker may be an indicator or result of a disease or condition. A biomarker may be an indicator of health. A biomarker may be an indicator of a genetic abnormality or inherited condition. A biomarker may be a circulating biomarker (e.g., found in a biological fluid such as blood). A biomarker may be a tissue biomarker (e.g., found in a solid organ such as liver or bone marrow). Non-limiting examples of biomarkers include nucleic acids, epigenetic modifications, proteins, peptides, antibodies, antibody fragments, lipids, fatty acids, sterols, polysaccharides, carbohydrates, viral particles, microbial particles. In some cases, biomarkers may even include whole cells or cell fragments.

As used herein, the term “genetic information” generally refers to one or more nucleic acid sequences. In some instances, genetic information may be a single nucleotide or amino acid. For example, genetic information could be the presence (or absence) of a single nucleotide polymorphism. Unless specified otherwise, the term “genetic information” may also refer to epigenetic modification patterns, gene expression data, and protein expression data. In some instances, the presence, absence or quantity of a biomarker provides genetic information. For instance, cholesterol levels may be indicative of a genetic form of hypercholesterolemia. Thus, genetic information should not be limited to nucleic acid sequences.

As used herein, the term “genomic equivalent” generally refers to the amount of DNA necessary to be present in a purified sample to guarantee that all genes will be present.

As used herein, the terms, “clinic,” “clinical setting,” “laboratory” or “laboratory setting” refer to a hospital, a clinic, a pharmacy, a research institution, a pathology laboratory, a or other commercial business setting where trained personnel are employed to process and/or analyze biological and/or environmental samples. These terms are contrasted with point of care, a remote location, a home, a school, and otherwise non-business, non-institutional setting.

As used herein, the term ‘about’ with reference to a number indicates a range including that number plus or minus 10% of that number. The term ‘about’ with reference to a numerical range refers to that range minus 10% of its lowest value and plus 10% of its greatest value.

As used herein, the term “specific to,” refers to a sequence or biomarker that is found only in, on or at the thing that the sequence or biomarker is specific to. For example, if a sequence is specific to a Y chromosome that means that it is only found on the Y chromosome and not on another chromosome.

As used in the specification and claims, the singular forms “a”, “an” and “the” include plural references unless the context clearly dictates otherwise. For example, the term “a sample” includes a plurality of samples, including mixtures thereof.

As used herein, the terms, “homolog,” “homologous,” “homology,” or “percent homology” describe sequence similarity of a first amino acid sequence or a nucleic acid sequence relative to a second amino acid sequence or a nucleic acid sequence. In some instances, homology can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application. In some cases, 2 or more sequences may be homologous if they share at least 20%, 25%, 30%. 35%, 40%, 45% 50%, 55%, 60% identity, 65%, 70%, 75%, 80%, 85%, 90%, 91%, 92%, 93%, 94%, 95%, 96%, 97%, 98%, 99% or higher identity when compared and aligned for maximum correspondence over a comparison window, or designated region as measured using one of the following sequence comparison algorithms or by manual alignment and visual inspection. In some cases, 2 or more sequences may be homologous if they share at most 20%, 25%, 30%, 35%, 40%, 45% 50%, 55%, 60% identity, 65%, 70%, 7:5%, 80%, 85%, 90%, 91%, 92%, 93%, 94%, 95%, 96%, 97%, 98%, 99% or higher identity. Preferably, the % identity or homology exists over a region that is at least 10 amino acids or nucleotides in length. In some cases, the % identity or homology exists over a region that is about 25 to about 100 amino acids or nucleotides in length. In some cases, the % identity or homology exists over a region that is about 50 to about 100 amino acids or nucleotides in length. In some cases, the % identity or homology exists over a region that is about 100 to about 1000 amino acids or nucleotides in length. In some cases, 2 or more sequences may be homologous and share at least 20% identity over at least 100 amino acids in a sequence. In some cases, 2 or more sequences may be homologous and share at least 50% identity over at least 100 amino acids in a sequence. For sequence comparison, generally one sequence acts as a reference sequence, to which test sequences may be compared. When using a sequence comparison algorithm, test and reference sequences may be entered into a computer, subsequent coordinates may be designated, if necessary, and sequence algorithm program parameters may be designated. Any suitable algorithm may be used, including but not limited to Smith-Waterman alignment algorithm, Viterbi, Bayesians, Hidden Markov and the like. Default program parameters can be used, or alternative parameters can be designated. The sequence comparison algorithm may then be used to calculate the percent sequence identities for the test sequences relative to the reference sequence, based on the program parameters. Any suitable algorithm may be used, whereby a percent identity is calculated. Some programs for example, calculate percent identity as the number of aligned positions that identical residues, divided by the total number of aligned positions. A “comparison window”, as used herein, includes reference to a segment of any one of the number of contiguous or non-contiguous positions which may range from 10 to 600 positions. In some cases the comparison window may comprise at least 10, 20, 50, 100, 200, 300, 400, 500, or 600 positions. In some cases the comparison window may comprise at most 10, 20, 50, 100, 200, 300, 400, 500, or 600 positions. In some cases the comparison window may comprise at least 50 to 200 positions, or at least 100 to at least 150 positions in which a sequence may be compared to a reference sequence of the same number of contiguous or non-contiguous positions after the two sequences are optimally aligned. Methods of alignment of sequences for comparison are well-known in the art. Optimal alignment of sequences for comparison can be conducted, e.g., by the local homology algorithm of Smith and Waterman, Adv. Appl. Math. 2:482 (1981), by the homology alignment algorithm of Needleman and Wunsch, J. Mot 48:443 (1970), by the search for similarity method of Pearson and Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al, eds. 1995 supplement)). In some cases, a comparison window may comprise any subset of the total alignment, either contiguous positions in primary sequence, adjacent positions in tertiary space but discontinuous in the primary sequence, or any other subset of 1 up to all residues in the alignment.

Throughout the application, there is recitation of the phrases “nucleic acid corresponding to a chromosome,” and “sequence corresponding to a chromosome,” e.g., “nucleic acid corresponding to a Y chromosome,” and “sequence corresponding to a Y chromosome.” As used herein, these phrases are intended to convey that the “nucleic acid corresponding to the chromosome” is represented by a nucleic acid sequence that is identical or homologous to a sequence found in that chromosome. The term “homologous” is described in the foregoing description.

Throughout the application, there is recitation of chromosome positions. These position numbers are in reference to Genome Build hg38 (UCSC) and GRCh38 (NCBI). A genome build may also be referred to in the art as a reference genome or reference assembly. It may be derived from multiple subjects. It is understood that there are multiple reference assemblies available and more reference assemblies may be produced over time. However, one skilled in the art would be able to determine the relative positions provided herein in another genome build or reference genome.

### DETAILED DESCRIPTION OF THE INVENTION

Genetic testing is traditionally performed in a laboratory or clinical setting. However, in many instances where genetic testing would be useful, access to a laboratory or clinic is unavailable or impractical. Thus, genetic tests that are operable at a point of need (e.g., locations remote from laboratories and clinics) are desirable. Genetic tests for operation at a point of need (e.g., home, school, farm) are preferably cost effective and simple for an untrained individual to perform. Genetic tests at point of need preferably require only small amounts of a biological sample. Traditionally, genetic testing requires a venous blood draw (phlebotomy) to obtain milliliters of blood containing enough DNA to be analyzed. However, a phlebotomy is not practical at a point of need. Ideally, a genetic test would only require amounts of blood achieved through the retrieval of capillary blood, e.g., via finger prick. This means point of need devices and methods for genetic testing need to be designed to function with low inputs of sample and a lower abundance of target molecules that are intended to be detected.

To exemplify the scaling challenge of analyzing circulating nucleic acids in capillary blood with an at-home or point of need device, one can use the analysis of cell-free DNA for the purpose of determining fetal gender. Traditionally, this is done from a venous blood draw of eight milliliters of blood. Up to four milliliters of plasma can be obtained from an eight milliliter blood draw. The amount of capillary blood from a finger prick (about 20 μl) is about 1/400 of a blood draw. On average, there are four thousand genome equivalents (or genome copies) in the form of circulating cell-free DNA represented in the four milliliters of plasma. Correspondingly, a finger prick amount will contain only about ten genome equivalents (or copies). In a pregnant woman, 10% of the circulating cell-free DNA on average is fetal in origin. Hence, the venous blood sample will have an average of four hundred fetal genome copies and the finger prick sample will only have an average of 1 fetal genome copy. As is eminent from these calculations, the assay performance (e.g., the sensitivity) of any assay targeting a single genome region will be limited by statistical sampling. For example, attempting to detect the fetal gender in a pregnant women from a finger prick amount of blood using a genomic region that is present only a single time and a single target region (e.g. using the SRY gene) would require at least 120 μl of capillary blood to have at least 1 copy of the target region represented in 95% of all samples tested.

In addition to accommodating low inputs of sample, it is desirable to have a genetic test that is capable of analyzing circulating cell free nucleic acids (DNA and RNA), e.g., circulating cell-free fetal DNA, circulating tumor DNA, circulating DNA from a transplanted donor organ, and circulating DNA released from a specific tissue as part of a health related issue, disease progression or treatment response. However, analysis of circulating cell-free nucleic acids is challenging due to their short half-life and therefore low abundance. In addition, circulating cell free nucleic acids in blood can be diluted by DNA released from white blood cells if care is not taken with the sample to avoid white blood cell lysis. White blood cell DNA creates background noise during detection of circulating cell-free nucleic acids, decreasing assay sensitivity and specificity.

Devices, systems, kits and methods disclosed herein overcome these challenges by combining gentle and efficient processing of small sample volumes (e.g., less than 1 ml) with a unique target region selection and assay design that takes advantage of the highly fragmented nature of circulating cell-free DNA (cfDNA). For example, devices, systems, kits and methods disclosed herein may provide reliable genetic information from a single finger prick. Devices, systems, kits and methods disclosed herein provide for analysis of multiple target regions along a target gene that are spaced far enough apart that the target regions are likely going to be physically separate when the target gene is fragmented in circulation. Thus, while the above described limits of statistical sampling exist for individual long DNA fragments that are traditionally analyzed in genetic testing, the sampling statistics change favorably for cfDNA fragments. While there may be a summation of only 1 genome equivalent present in a capillary blood sample, there are many individual cfDNA fragments. Consequently, sensitive amplification can be achieved from low input amounts if multiple target regions on separate cell free fragments are analyzed.

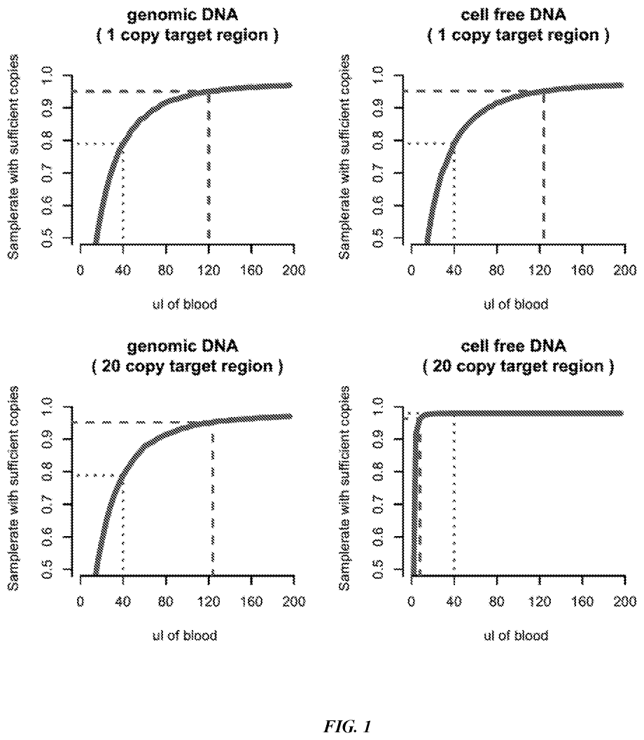

FIG. 1 shows the success rate of detecting multiple target regions when detecting or amplifying genomic DNA versus circulating cell-free DNA. If only a single copy region or target sequence is amplified, a relatively high amount of blood is required to make sure that at least 95% of samples have 1 copy (which is the minimum required to have any chance of amplification or detection). If a multi-copy region or multiple copies of a target sequence are amplified (e.g., 20 copies), but they are present in the sample as long DNA molecules such as genomic DNA, a relatively high amount of blood is still required to detect at least 1 copy of the target sequence. In contrast, the success rate of amplification and detection increases dramatically for target regions in cell free DNA from relatively low amounts of sample. By way of non-limiting example, a relatively low amount would be the amount of blood from a finger prick and a relatively high amount would be the amount of blood from a phlebotomy.

As an example, if twenty target regions are present along a genomic region and they are spaced far enough apart that they can be independently analyzed and detected when the DNA is fragmented, the input volume required to have at least 1 target region in 95% of all samples changes from 140 μl (genomic DNA) to less than 25 μl (cfDNA), significantly increasing sensitivity. In some instances, the target regions contain identical sequences or similar sequences. These target regions may be referred to as copies. A non-limiting example for this is the TTTSY region on chromosome Y, which has about 20 homologs. This is an example of a highly repetitive region, which is further described herein. Advantageously, all twenty regions may be amplified with the same primer pair. The concentration of the fragments containing the target region is twenty times higher than a non-fragmented Y chromosome or a fragment of the Y chromosome that is not repeated. Thus, there will be twenty times more signal from the TTTSY region than one would get from a non-fragmented Y chromosome or a region of the Y chromosome that is not repeated. Another way to look at this is that it will be twenty times more likely that one copy of the target region is present in a low volume of sample than a non-target region that is not repeated or does not share some detectable commonality with another region.

In other instances, target regions may not share similar sequences, but share another characteristic such as a similar epigenetic status. For example, the multiple target regions may have different sequences but they are all hyper-methylated. Regardless of the specific types of epigenetic modifications, the sites of epigenetic modification are spaced appropriately to leverage the fragmentation pattern of circulating cell free DNA which produces many circulating cfDNA fragments of which at least one can be detected in a small volume. By way of non-limiting example, selected target regions that are distant enough from each other to be on separate cfDNA fragments and are all hyper-methylated when a subject has cancer can be detected with bisulfite sequencing. In a small sample volume (e.g., a finger prick of blood), the likelihood that all of these fragments are present (which is equivalent to non-fragmented DNA) is low, but the likelihood that at least one fragment is present is high, and the cancer can be detected.

In yet other instances, the target regions may not contain similar sequences and may not contain similar epigenetic status. In this case, detection may require multiple primer sets or library preparation followed by amplification with universal primers to detect several distinct target regions. By way of non-limiting example, the detection of a fetal RHD gene in an RHD negative pregnant mother could be achieved from a finger prick amount of blood by using multiple sets of primers to detect multiple different exons of the RHD gene in cell-free fetal DNA fragments. By using twenty sets of primers, the same sensitivity that was achieved using twenty repeat sequences in the example of the TTTSY region above can be achieved. Sensitivity can be increased by choosing primers that amplify regions that are physically distant in the RHD gene and therefore likely to be present on different cell-free DNA fragments. Detecting a fetal RHD gene in an RHD negative pregnant mother is important to prevent hemolytic disease of a newborn by the mother having antibodies against the child's blood. RHD testing is currently performed today from full blood draws (eight milliliters of blood) to achieve the appropriate reliable results. This volume is believed to be necessary to achieve reliable results because it is based on the likelihood that the entire RHD gene will be present in the sample. Based on this assumption, the likelihood of getting the whole RHD gene in a finger prick amount of blood is low and would easily lead to false negative results.

Regardless of how target regions are chosen, these regions are present in the sample as individual biomarkers when amplification or detection is performed on cell free fragmented DNA. The concentration of the fragments containing the target region is greater than the corresponding non-fragmented DNA or a fragment that cannot be assayed as a group. Thus, there will be more signal from the target region than one would get from non-fragmented DNA or from assaying for one copy of the target region. One will be much more likely to detect a target region present in a low volume of sample than a non-target region that is not repeated or does not share some commonality with another region. By assaying multiple target regions in multiple DNA fragments, assay sensitivity is increased relative to traditional testing.

Blood is a reliable source of cell-free nucleic acids. Most methods for analyzing cell-free nucleic acids from blood involve isolating the plasma or serum fraction containing the cell-free nucleic acids. Devices, systems, kits and methods disclosed herein allow for gentle processing of a blood sample at a point of need. This may avoid, prevent or reduce white blood cell lysis. Devices, systems, kits and methods disclosed herein allow for rapid processing of a blood sample at a point of need. This avoids elongated storage and shipment of samples that can lead to blood cell lysis. In some instances, devices disclosed herein perform integrated separation, e.g. immediate isolation of plasma through filtration, to avoid, reduce or prevent cell lysis. Immediate separation of cells from cfDNA may be desirable when a reagent (e.g., probe, primer, antibody) or detection method does not provide much specificity. In some instances, methods are performed with whole blood in an effort to avoid any white blood cell lysis. When relatively higher specificity can be achieved, analysis from whole blood may be more desirable.

In addition to requiring only small volumes of samples, devices, systems, kits and methods disclosed herein are highly desirable for at least the following reasons. Devices, systems, kits and methods disclosed herein generally require little to no technical training. Thus, the costs of performing genetic testing is reduced relative to the cost of testing performed by trained personnel, and the test is available to subjects who do not have access to trained personnel. Furthermore, results may be obtained within minutes (e.g., less than an hour). This may be especially important when testing for an infection. An individual or animal testing positive for an infection may be isolated and treated quickly, preventing the spread of infection. Moreover, results may be obtained privately. In some cases, only the patient that is being tested is privy to the genetic information obtained. Devices, systems and kits disclosed herein are generally lightweight and handheld, making them suitable and accessible to remote locations. Thus, they may be employed at home, in a school, on a battlefield, on a farm, or any other site where it would be impractical or inconvenient to visit a laboratory or clinical setting. Furthermore, since the sample may be analyzed at the point of care, the sample does not need to be stored or shipped, reducing the risk of sample degradation and misidentification (e.g., sample swapping).

In some instances, devices, systems, kits and methods disclosed herein are desirable because the genetic information can be kept private to the user. In fact, even the use of the device can be kept private. Alternatively, devices, systems, kits and methods are configured to share information with others or can be easily adapted by the user to share information (e.g., turning on a Bluetooth signal). For example, information may be easily shared with a nurse or doctor. In some instances, the device or system can send/share test results through a secure portal or application programming interface (API) to a medical practitioner or staff at an office or hospital. In some instances, the user may choose to share information with the medical practitioner in person after receiving the result. In some instances, the information may even be shared in real-time. For example, gender determination may be shared in real time with family and friends via communication components of the devices, systems, and kits. This kind of communication would be desirable for couples or families that are split up, for example, by military commitments, employment obligations, migration policies, or health issues. In the example of gender determination provided above, a pregnant woman, in the privacy of her own home, may be able to videoconference (e.g., Skype, Face Time) with her husband overseas to simultaneously determine the gender of their baby.

There are myriad applications for the devices, systems, kits, and methods disclosed herein. Devices, systems, kits and methods disclosed herein allow for diagnosing and monitoring medical conditions. Non-limiting examples of medical conditions include autoimmune conditions, metabolic conditions, cancer, and neurological conditions. Devices, systems, kits and methods disclosed herein allow for personalized medicine, including microbiome testing, determining an appropriate personal medical dosage and/or detecting a response to a drug or dose thereof. Devices, systems, kits and methods disclosed herein also allow for detecting a food allergen and detecting food/water contamination. Devices, systems, kits and methods disclosed herein provide for detecting an infection by a pathogen and/or a subject's resistance to drugs that could be used to treat the infection. In almost all cases, there is little to no need for technical training or large, expensive laboratory equipment.

### I. Devices, Systems and Kits

In some aspects, disclosed herein are devices, systems and kits for obtaining genetic information. As described herein, devices, systems and kits disclosed herein allow a user to collect and test a sample at a location of choice to determine the presence and/or quantity of a target analyte in the sample. The sample may be a sample from a subject, such as a biological fluid (e.g., blood, urine). The sample may be an environmental sample (e.g., waste water, soil, food/beverage).

In some instances, devices, systems, and kits disclosed herein comprise a sample purifier that removes at least one biological component from a biological fluid sample of a subject; at least one nucleic acid amplification reagent; at least one oligonucleotide comprising a sequence corresponding to a region of interest, wherein the at least one oligonucleotide and nucleic acid amplification reagent are capable of producing an amplification product; and at least one of a detection reagent or a signal detector for detecting the amplification product. In some instances, the at least one biological component is a cell, a cell fragment, a microparticle, an exosome, a nucleosome, a protein, or a combination thereof. By way of non-limiting example, the subject may be a pregnant subject and the region of interest may be a region on a Y chromosome. By way of non-limiting example, a region of interest may be in a gene implicated in a cancer, an autoimmune condition, a neurological disorder, a metabolic disorder, a cardiovascular disease, immunity (e.g., infection susceptibility or resistance), and drug metabolism. A gene implicated in a disease, disorder or condition is considered a gene that when mutated, deleted, copied, epigenetically modified, under- or overexpressed, changes at least one of a symptom, outcome, duration, or onset of the disease, disorder or condition.

In some instances, devices, systems, and kits disclosed herein comprise a sample purifier that removes a cell from a biological fluid sample of a subject; at least one nucleic acid amplification reagent; at least one oligonucleotide comprising a sequence corresponding to a region of interest, wherein the at least one oligonucleotide and nucleic acid amplification reagent are capable of producing an amplification product; and at least one of a detection reagent or a signal detector for detecting the amplification product. In some instances, devices, systems, and kits disclosed herein comprise a miniaturized digital nucleic acid amplification platform. By way of non-limiting example, the miniaturized nucleic acid amplification platform may be located on a chip within a device disclose herein, thereby keeping the entire device or system to a handheld size (e.g., similar to a cell phone). In some instances, the miniaturized nucleic acid amplification platform incorporates or is accompanied by digital output for ease of test result display.

In some instances, devices, systems, and kits disclosed herein comprise a sample purifier that removes a cell from a biological sample of a subject; a nucleic acid sequencer for obtaining sequencing reads from nucleic acids in the biological sample; and at least one of a detection reagent or a signal detector for detecting the sequencing reads. Non-limiting examples of a nucleic acid sequencer include next generation sequencing machines, nanopore sequencers, single molecule counters (e.g., counting sequences that are bar-coded/tagged).

In general, devices, systems, and kits disclosed herein, integrate multiple functions, e.g., purification, amplification, detection, and determination of the target analyte (including amplification products thereof), and combinations thereof. In some instances, the multiple functions are carried out within a single assay assembly unit or a single device. In some instances, all of the functions occur outside of the single unit or device. In some instances, at least one of the functions occurs outside of the single unit or device. In some instances, only one of the functions occurs outside of the single unit or device. In some instances, the sample purifier, nucleic acid amplification reagent, oligonucleotide, and detection reagent or component are housed in a single device. In general, devices, systems, and kits disclosed herein comprise a display, a connection to a display, or a communication to a display for relaying information about the biological sample to one or more people.

In some instances, devices, systems and kits comprise an additional component disclosed herein. Non-limiting examples of an additional component include a sample transportation compartment, a sample storage compartment, a sample and/or reagent receptacle, a temperature indicator, an electronic port, a communication connection, a communication device, a sample collection device, and a housing unit. In some instances, the additional component is integrated with the device. In some instances, the additional component is not integrated with the device. In some instances, the additional component is housed with the sample purifier, nucleic acid amplification reagent, oligonucleotide, and detection reagent or component in a single device. In some instances, the additional component is not housed within the single device.

In some instances, devices, systems and kits comprise a receptacle for receiving the biological sample. The receptacle may be configured to hold a volume of a biological sample between 1 μl and 1 ml. The receptacle may be configured to hold a volume of a biological sample between 1 μl and 500 μl. The receptacle may be configured to hold a volume of a biological sample between 1 μl and 200 μl. The receptacle may have a defined volume that is the same as a suitable volume of sample for processing and analysis by the rest of the device/system components. This would preclude the need for a user of the device, system or kit to measure out a specified volume of the sample. The user would only need to fill the receptacle and thereby be assured that the appropriate volume of sample had been delivered to the device/system. In some instances, devices, systems and kits do not comprise a receptacle for receiving the biological sample. In some instances, the sample purifier receives the biological sample directly. Similar to the description above for the receptacle, the sample purifier may have a defined volume that is suitable for processing and analysis by the rest of the device/system components.

In general, devices, systems, and kits disclosed herein are intended to be used entirely at point of care. However, in some instances, the user may want to preserve or send the analyzed sample to another location (e.g., lab, clinic) for additional analysis or confirmation of results obtained at point of care. In some instances, devices, systems and kits comprise a transport compartment or storage compartment for these purposes. The transport compartment or storage compartment may be capable of containing a biological sample, a component thereof, or a portion thereof. The transport compartment or storage compartment may be capable of containing the biological sample, portion thereof, or component thereof, during transit to a site remote to the immediate user. Non-limiting examples of a site remote to the immediate user may be a laboratory or a clinic when the immediate user is at home. In some instances, the home does not have a machine or additional device to perform an additional analysis of the biological sample. The transport compartment or storage compartment may be capable of containing a product of a reaction or process that occurs in the device. In some instances, the product of the reaction or process is a nucleic acid amplification product or a reverse transcription product. In some instances, the product of the reaction or process is a biological sample component bound to a binding moiety described herein. The biological sample component may comprise a nucleic acid, cell fragment, an extracellular vesicle, a protein, a peptide, a sterol, a lipid, a vitamin, or glucose, any of which may be analyzed at a remote location to the user. In some instances, the transport compartment or storage compartment comprises an absorption pad, a paper, a glass container, a plastic container, a polymer matrix, a liquid solution, a gel, a preservative, or a combination thereof. In some instances, the device, system or kit comprises a stabilizer (chemical or structure (e.g., matrix)) that reduces enzymatic activity during storage and/or transportation.

Generally, devices and systems disclosed herein are portable for a single person. In some instances, devices and systems are handheld. In some instances, devices and systems have a maximum length, maximum width or maximum height. In some instances, devices and systems are housed in a single unit having a maximum length, maximum width or maximum height. In some instances the maximum length is not greater than 12 inches. In some instances the maximum length is not greater than 10 inches. In some instances the maximum length is not greater than 8 inches. In some instances the maximum length is not greater than 6 inches. In some instances the maximum width is not greater than 12 inches. In some instances the maximum width is not greater than 10 inches. In some instances the maximum width is not greater than 8 inches. In some instances the maximum width is not greater than 6 inches. In some instances the maximum width is not greater than 4 inches. In some instances the maximum height is not greater than 12 inches. In some instances the maximum height is not greater than 10 inches. In some instances the maximum height is not greater than 8 inches. In some instances the maximum height is not greater than 6 inches. In some instances the maximum height is not greater than 4 inches. In some instances the maximum height is not greater than 2 inches. In some instances the maximum height is not greater than 1 inch.

### Sample Collection

In some instances, devices, systems and kits disclosed herein comprise a sample collector. In some instances, the sample collector is provided separately from the rest of the device, system or kit. In some instances, the sample collector is physically integrated with the device, system or kit, or a component thereof. In some instances, the sample collector is integrated with a receptacle described herein. In some instances, the sample collector may be a cup, tube, capillary, or well for applying the biological fluid. Biological fluids are described herein and throughout. In some instances, the sample collector may be a cup for applying urine. In some instances, the sample collector may comprise a pipet for applying urine in the cup to the device, system or kit. In some instances, the sample collector may be a capillary integrated with a device disclosed herein for applying blood. In some instances, the sample collector may be tube, well, pad or paper integrated with a device disclosed herein for applying saliva. In some instances, the sample collector may be pad or paper for applying sweat.

In some instances, devices, systems and kits disclosed herein comprise a transdermal puncture device. Non-limiting examples of transdermal puncture devices are needles and lancets. In some instances, the sample collector comprises the transdermal puncture device. In some instances, devices, systems and kits disclosed herein comprise a microneedle, microneedle array or microneedle patch. In some instances, devices, systems and kits disclosed herein comprise a hollow microneedle. By way of non-limiting example, the transdermal puncture device is integrated with a well or capillary so that as the subject punctures their finger, blood is released into the well or capillary where it will be available to the system or device for analysis of its components. In some instances, the transdermal puncture device is a push button device with a needle or lancet in a concave surface. In some instances, the needle is a microneedle. In some instances, the transdermal puncture device comprises an array of microneedles. By pressing an actuator, button or location on the non-needle side of the concave surface, the needle punctures the skin of the subject in a more controlled manner than a lancet. Furthermore, the push button device may comprise a vacuum source or plunger to help draw blood from the puncture site.

In some instances, devices disclosed herein comprise a transdermal puncture device, wherein the device stabilizes blood. The device or a portion thereof (e.g., storage/shipping compartment, filter pad or paper) containing the stabilized blood may be sent to a laboratory for additional processing and analysis. In some instances, devices disclosed herein comprise a transdermal puncture device, wherein the device comprises a sample purifier that separates plasma from red blood cells. The device or a portion thereof containing the plasma may be sent to a laboratory for additional processing and analysis.

### Sample Purification

Disclosed herein are devices, systems and kits that comprise a sample purifier to remove an unwanted substance or non-target component of a biological sample, thereby modifying the sample. Depending on the source of the biological sample, unwanted substances can include, but are not limited to, proteins (e.g., antibodies, hormones, enzymes, serum albumin, lipoproteins), free amino acids and other metabolites, microvesicles, nucleic acids, lipids, electrolytes, urea, urobilin, pharmaceutical drugs, mucous, bacteria, and other microorganisms, and combinations thereof. In some instances, the sample purifier separates components of a biological sample disclosed herein. In some instances, sample purifiers disclosed herein remove components of a sample that would inhibit, interfere with or otherwise be detrimental to the later process steps such as nucleic acid amplification or detection. In some instances, the resulting modified sample is enriched for target analytes. This can be considered indirect enrichment of target analytes. Alternatively or additionally, target analytes may be captured directly, which is considered direct enrichment of target analytes.

In some instances, the sample purifier comprises a separation material for removing unwanted substances other than patient cells from the biological sample. Useful separation materials may include specific binding moieties that bind to or associate with the substance. Binding can be covalent or noncovalent. Any suitable binding moiety known in the art for removing a particular substance can be used. For example, antibodies and fragments thereof are commonly used for protein removal from samples. In some instances, a sample purifier disclosed herein comprises a binding moiety that binds a nucleic acid, protein, cell surface marker, or microvesicle surface marker in the biological sample. In some instances, the binding moiety comprises an antibody, antigen binding antibody fragment, a ligand, a receptor, a peptide, a small molecule, or a combination thereof.

In some instances, sample purifiers disclosed herein comprise a filter. In some instances, sample purifiers disclosed herein comprise a membrane. Generally the filter or membrane is capable of separating or removing cells, cell particles, cell fragments, blood components other than cell-free nucleic acids, or a combination thereof, from the biological samples disclosed herein.

In some instances, the sample purifier facilitates separation of plasma from cellular components of a blood sample before starting a molecular amplification reaction. Plasma separation may be achieved by several different methods such as centrifugation, sedimentation or filtration. In some instances, sample purifiers disclosed herein comprise a filter. In some instances, the sample purifier employs vertical filtration. Vertical filtration is filtration driven by a capillary force to separate the plasma from the blood. In some instances, the sample purifier comprises a filter matrix for receiving whole blood, the filter matrix having a pore size that is prohibitive for cells to pass through, while plasma can pass through the filter matrix uninhibited. In some instances, the filter matrix combines a large pore size at the top with a small pore size at the bottom of the filter, which leads to very gentle treatment of the cells preventing cell degradation or lysis, during the filtration process. This is advantageous because cell degradation or lysis would result in release of nucleic acids from blood cells or maternal cells that would contaminate target cell-free nucleic acids. Non-limiting examples of such filters include Pall Vivid™ GR membrane, Munktell Ahlstrom filter paper (see, e.g., WO2017017314), TeraPore Technologies filters.

In some instances, the sample purifier comprises an appropriate separation material, e.g., a filter or membrane that removes unwanted substances from a biological sample without removing cell-free nucleic acids. In some instances, the separation material separates substances in the biological sample based on size, for example, the separation material has a pore size that excludes a cell but is permeable to cell-free nucleic acids. Therefore, when the biological sample is blood, the plasma and/or serum can move more rapidly than a blood cell through the separation material in the sample purifier, and the plasma or serum containing any cell-free nucleic acids permeates the holes of the separation material. In some instances, the biological sample is blood, and the cell that is slowed and/or trapped in the separation material is a red blood cell, a white blood cell, or a platelet. In some instances, the cell is from a tissue that contacted the biological sample in the body, including, but not limited to, a bladder or urinary tract epithelial cell (in urine), or a buccal cell (in saliva). In some instances, the cell is a bacterium or other microorganism.

In some instances, the sample purifier is capable of slowing and/or trapping a cell without damaging the cell, thereby avoiding the release of cell contents including cellular nucleic acids and other proteins or cell fragments that could interfere with subsequent evaluation of the cell-free nucleic acids. This can be accomplished, for example, by a gradual, progressive reduction in pore size along the path of a lateral flow strip or other suitable assay format, to allow gentle slowing of cell movement, and thereby minimize the force on the cell. In some instances, at least 95%, at least 98%, at least 99%, or 100% of the cells in a biological sample remain intact when trapped in the separation material. In addition to or independently of size separation, the separation material can trap or separate unwanted substances based on a cell property other than size, for example, the separation material can comprise a binding moiety that binds to a cell surface marker. In some instances, the binding moiety is an antibody or antigen binding antibody fragment. In some instances, the binding moiety is a ligand or receptor binding protein for a receptor on a blood cell or microvesicle.

In some instances devices, systems, and kits disclosed herein employ vertical filtration, driven by capillary force to separate a component or fraction from a sample (e.g., plasma from blood). By way of non-limiting example, vertical filtration may comprise gravitation assisted plasma separation. A high-efficiency superhydrophobic plasma separator is described, e.g., by Liu et al., A High Efficiency Superhydrophobic Plasma Separation, Lab Chip 2015.

The sample purifier may comprise a lateral filter (e.g., sample does not move in a gravitational direction or the sample moves perpendicular to a gravitational direction). The sample purifier may comprise a vertical filter (e.g., sample moves in a gravitational direction). The sample purifier may comprise vertical filter and a lateral filter. The sample purifier may be configured to receive a sample or portion thereof with a vertical filter, followed by a lateral filter. The sample purifier may be configured to receive a sample or portion thereof with a lateral filter, followed by a vertical filter. In some instances, a vertical filter comprises a filter matrix. In some instances, the filter matrix of the vertical filter comprises a pore with a pore size that is prohibitive for cells to pass through, while plasma can pass the filter matrix uninhibited. In some instances, the filter matrix comprises a membrane that is especially suited for this application because it combines a large pore size at the top with a small pore size at the bottom of the filter, which leads to very gentle treatment of the cells preventing cell degradation during the filtration process.

In some instances, devices disclosed herein comprise a separation material that moves, draws, pushes, or pulls the biological sample through the sample purifier, filter and/or membrane. In some instances, the material is a wicking material. Examples of appropriate separation materials used in the sample purifier to remove cells include, but are not limited to, polyvinylidene difluoride, polytetrafluoroethylene, acetylcellulose, nitrocellulose, polycarbonate, polyethylene terephthalate, polyethylene, polypropylene, glass fiber, borosilicate, vinyl chloride, silver. Suitable separation materials may be characterized as preventing passage of the cells. In some instances, the separation material can prevent passage of red blood cells. In some instances, the separation material is a hydrophobic filter, for example a glass fiber filter, a composite filter, for example Cytosep (e.g., Ahlstrom Filtration or Pall Specialty Materials, Port Washington, N.Y.), or a hydrophilic filter, for example cellulose (e.g., Pall Specialty Materials). In some instances, whole blood can be fractionated into red blood cells, white blood cells and serum components for further processing according to the methods devices, systems and kits disclosed herein using a commercially available kit (e.g., Arrayit® Blood Card Serum Isolation Kit, Cat. ABCS, Arrayit Corporation, Sunnyvale, Calif.).

In some instances the sample purifier comprises at least one filter or at least one membrane characterized by at least one pore size. In some instances, the sample purifier comprises multiple filters and/or membranes, wherein the pore size of at least a first filter or membrane differs from a second filter or membrane. In some instances, at least one pore size of at least one filter/membrane is about 0.05 microns to about 10 microns. In some instances, at least one pore size of at least one filter/membrane is about 0.05 microns to about 8 microns. In some instances, at least one pore size of at least one filter/membrane is about 0.05 microns to about 6 microns. In some instances, at least one pore size of at least one filter/membrane is about 0.05 microns to about 4 microns. In some instances, at least one pore size of at least one filter/membrane is about 0.05 microns to about 2 microns. In some instances, at least one pore size of at least one filter/membrane is about 0.05 microns to about 1 micron.

Gentle sample purifiers, such as those comprising a filter matrix, a vertical filter, a wicking material, or a membrane with pores that do not allow passage of cells, are particularly useful for analyzing cell-free nucleic acids. For example, prenatal applications of cell-free fetal nucleic acids in maternal blood are presented with the additional challenge of analyzing cell-free fetal nucleic acids in the presence of cell-free maternal nucleic acids, the latter of which create a large background signal to the former. By way of non-limiting example, a sample of maternal blood may contain about 500 to 750 genome equivalents of total cell free DNA (maternal and fetal) per milliliter of whole blood when the sample is obtained without cell lysis or other cell disruption caused by the sample collection method. The fetal fraction in blood sampled from pregnant women may be around 10%, about 50 to 75 genome equivalents per ml. The process of obtaining cell-free nucleic acids usually involves obtaining plasma from the blood. If not performed carefully, maternal white blood cells may be destroyed, releasing additional cellular nucleic acids into the sample, creating a lot of background noise to the fetal cell-free nucleic acids. The typical white cell count is around 4*10\hat{ }6 to 10*10\hat{ }6 cells per ml of blood and therefore the available nuclear DNA is around 4,000 to 10,000 times higher than the overall cell-free DNA (cfDNA). Consequently, even if only a small fraction of maternal white blood cells is destroyed, releasing nuclear DNA into the plasma, the fetal fraction is reduced dramatically. For example, a white cell degradation of 0.01% may reduce the fetal fraction from 10% to about 5%. Devices, systems, and kits disclosed herein aim to reduce these background signals.

In some instances, devices, systems and kits disclosed herein comprise a binding moiety for producing a modified sample depleted of cells, cell fragments, nucleic acids or proteins that are unwanted or of no interest. In some instances, devices, systems and kits disclosed herein comprise a binding moiety for reducing cells, cell fragments, nucleic acids or proteins that are unwanted or of no interest, in a biological sample. In some instances, devices, systems and kits disclosed herein comprise a binding moiety for producing a modified sample enriched with target cell, target cell fragments, target nucleic acids or target proteins.

In some instances, devices, systems and kits disclosed herein comprise a binding moiety capable of binding a nucleic acid, a protein, a peptide, a cell surface marker, or microvesicle surface marker. In some instances, devices, systems and kits disclosed herein comprise a binding moiety for capturing an extracellular vesicle or extracellular microparticle in the biological sample. In some instances, the extracellular vesicle contains at least one of DNA and RNA. In some instances, devices, systems and kits disclosed herein comprise reagents or components for analyzing DNA or RNA contained in the extracellular vesicle. In some instances, the binding moiety comprises an antibody, antigen binding antibody fragment, a ligand, a receptor, a protein, a peptide, a small molecule, or a combination thereof.

In some instances, devices, systems and kits disclosed herein comprise a binding moiety capable of interacting with or capturing an extracellular vesicle that is released from a cell. In some instances, the cell is a fetal cell. In some instances, the cell is a placental cell. The fetal cell or the placental cell may be circulating in a biological fluid (e.g., blood) of a female pregnant subject. In some instances, the extracellular vesicle is released from an organ, gland or tissue. By way of non-limiting example, the organ, gland or tissue may be diseased, aging, infected, or growing. Non-limiting examples of organs, glands and tissues are brain, liver, heart, kidney, colon, pancreas, muscle, adipose, thyroid, prostate, breast tissue, and bone marrow.

In some instances, devices, systems and kits disclosed herein disclosed are capable of capturing and discarding an extracellular vesicle or extracellular microparticle from a maternal sample to enrich the sample for fetal/placental nucleic acids. In some instances, the extracellular vesicle is fetal/placental in origin. In some instances, the extracellular vesicle originates from a fetal cell. In some instances, the extracellular vesicle is released by a fetal cell. In some instances, the extracellular vesicle is released by a placental cell. The placental cell may be a trophoblast cell. In some instances, devices, systems and kits disclosed herein comprise a cell-binding moiety for capturing placenta educated platelets, which may contain fetal DNA or RNA fragments. These can be captured/enriched for with antibodies or other methods (low speed centrifugation). In such instances, the fetal DNA or RNA fragments may be analyzed as described herein to determine or indicate chromosomal information (e.g., gender). Alternatively or additionally, devices, systems and kits disclosed herein comprise a binding moiety for capturing an extracellular vesicle or extracellular microparticle in the biological sample that comes from a maternal cell.

In some instances, the binding moiety is attached to a solid support, wherein the solid support can be separated from the rest of the biological sample or the biological sample can be separated from the solid support, after the binding moiety has made contact with the biological sample. Non-limiting examples of solid supports include a bead, a nanoparticle, a magnetic particle, a chip, a microchip, a fibrous strip, a polymer strip, a membrane, a matrix, a column, a plate, or a combination thereof.

Devices, systems and kits disclosed herein may comprise a cell lysis reagent. Non-limiting examples of cell lysis reagents include detergents such as NP-40, sodium dodecyl sulfate, and salt solutions comprising ammonium, chloride, or potassium. Devices, systems and kits disclosed herein may have a cell lysis component. The cell lysis component may be structural or mechanical and capable of lysing a cell. By way of non-limiting example, the cell lysis component may shear the cells to release intracellular components such as nucleic acids. In some instances, devices, systems and kits disclosed herein do not comprise a cell lysis reagent. Some devices, systems and kits disclosed herein are intended to analyze cell-free nucleic acids.

### Nucleic Acid Amplification

Generally, devices, systems and kits disclosed herein are capable of amplifying a nucleic acid. In some instances, the nucleic acid comprises DNA. DNA may be genomic. DNA may be mitochondrial. In some instances, the nucleic acid comprises RNA. In some instances, the nucleic acid comprises cell-free DNA. In some instances, the nucleic acid comprises cell-free genomic DNA. In some instances, the devices, systems and kits disclosed herein comprise a reverse transcriptase enzyme to produce complementary DNA (cDNA) from RNA in biological samples disclosed herein, wherein the cDNA can be amplified and/or analyzed similarly to genomic DNA as described herein. The RNA may comprise circulating cell-free RNA. The nucleic acid may be a cell-free fetal nucleic acid.

In some instances, devices, systems, kits, and methods disclosed herein comprise at least one nucleic acid amplification reagent, or use thereof. Non-limiting examples of nucleic acid amplification reagents are polymerases, primers, nucleic acid amplification buffers, and free nucleotides.

A traditional polymerase chain reaction requires thermocycling. This would be possible, but inconvenient for a typical at-home user without a thermocycler machine. In some instances, devices, systems and kits disclosed herein are capable of amplifying a nucleic acid without changing the temperature of the device or system or a component thereof. In some instances, devices, systems and kits disclosed herein are capable of amplifying a nucleic acid isothermally. Non-limiting examples of isothermal amplification are as follows: loop-mediated isothermal amplification (LAMP), strand displacement amplification (SDA), helicase dependent amplification (HDA), nicking enzyme amplification reaction (NEAR), and recombinase polymerase amplification (RPA). Thus, devices, systems and kits disclosed herein may comprise reagents necessary to carry out an isothermal amplification. Non-limiting examples of isothermal amplification reagents include recombinase polymerases, single-strand DNA-binding proteins, and strand-displacing polymerases. Generally, isothermal amplification using recombinase polymerase amplification (RPA) employs three core enzymes, recombinase, single-strand DNA-binding protein, and strand-displacing polymerase, to (1) pair oligonucleotide primers with homologous sequence in DNA, (2) stabilize displaced DNA strands to prevent primer displacement, and (3) extend the oligonucleotide primer using a strand displacing DNA polymerase. Using paired oligonucleotide primers, exponential DNA amplification can take place with incubation at room temperature (optimal at 37° C.).

In some instances, devices, systems and kits disclosed herein are capable of amplifying a nucleic acid at a single temperature. In some instances, devices, systems and kits disclosed herein may advantageously be operated at room temperature. In some instances, devices, systems and kits disclosed herein are capable of amplifying a nucleic acid isothermally at temperatures ranging from about 20° C. to about 65° C. In some instances, devices, systems and kits disclosed herein are capable of amplifying a nucleic acid isothermally at about 23° C. to about 27° C. In some instances, devices, systems and kits disclosed herein are capable of amplifying a nucleic acid at not more than two temperatures. In some instances, devices, systems and kits disclosed herein are capable of amplifying a nucleic acid at not more than three temperatures. In some instances, devices, systems and kits disclosed herein only require initially heating one reagent or component of the device, system or kit.

In some instances, devices, systems, kits, and methods disclosed herein comprise a hybridization probe with an abasic site, a fluorophore and a quencher to monitor amplification. Endo or exo-nucleases such as Endonuclease IV or Exonuclease III may be included to cleave the abasic site and release the quencher to allow fluorescent excitation. In some instances, amplification products are detected or monitored via lateral flow by attaching a capture molecule (e.g. Biotin) to one of the amplification primers and labeling a hybridization primer with a 5′-antigenic molecule (e.g. fluorescein derivative FAM) for capture to allow for detection. As such, in some instances, devices, systems, kits, and methods disclosed herein provide for detection of nucleic acids and amplification products on a lateral flow device. Lateral flow devices are described herein.

In some instances, devices, systems and kits disclosed herein comprise at least one nucleic acid amplification reagent and at least one oligonucleotide primer capable of amplifying a first sequence in a genome and a second sequence in a genome, wherein the first sequence and the second sequence are similar, and wherein the first sequence is physically distant enough from the second sequence such that the first sequence is present on a first cell-free nucleic acid of the subject and the second sequence is present on a second cell-free nucleic acid of the subject. In some instances, the at least two sequences are immediately adjacent. In some instances, the at least two sequences are separated by at least one nucleotide. In some instances, the at least two sequences are separated by at least two nucleotides. In some instances, the at least two sequences are separated by at least about 5, at least about 10, at least about 15, at least about 20, at least about 30, at least about 40, at least about 50, or at least about 100 nucleotides. In some instances, the at least two sequences are at least about 50% identical. In some instances, the at least two sequences are at least about 60% identical, at least about 60% identical, at least about 60%, at least about 70%, at least about 80%, at least about 90%, at least about 95%, at least about 99%, or 100% identical. In some instances, the first sequence and the second sequence are each at least 10 nucleotides in length. In some instances, the first sequence and the second sequence are each at least about 10, at least about 15, at least about 20, at least about 30, at least about 50, or at least about 100 nucleotides in length. In some instances, the first sequence and the second sequence are on the same chromosome. In some instances, the first sequence is on a first chromosome and the second sequence is on a second chromosome. In some instances, the first sequence and the second sequence are in functional linkage. For example, all CpG sites in the promotor region of gene AOX1 show the same hypermethylation in prostate cancer, so these sites are in functional linkage because they functionally carry the same information but are located one or more nucleotides apart.

In some instances, devices, systems and kits disclosed herein comprise at least one of an oligonucleotide probe or oligonucleotide primer that is capable of annealing to a strand of a cell-free nucleic acid, wherein the cell-free nucleic acid comprises a sequence corresponding to a region of interest or a portion thereof. In some instances, the region of interest is a region of a Y chromosome. In some instances, the region of interest is a region of an X chromosome. In some instances, the region of interest is a region of an autosome. In some instances, the region of interest, or portion thereof, comprises a repeat sequence as described herein that is present in a genome more than once.

In some instances, a region of interest disclosed herein is about 10 nucleotides to about 1,000,000 nucleotides in length. In some instances, the region of interest is at least 10 nucleotides in length. In some instances, the region of interest is at least 100 nucleotides in length. In some instances, the region is at least 1000 nucleotides in length. In some instances, the region of interest is about 10 nucleotides to about 500,000 nucleotides in length. In some instances, the region of interest is about 10 nucleotides to about 300,000 nucleotides in length. In some instances, the region of interest is about 100 nucleotides to about 1,000,000 nucleotides in length. In some instances, the region of interest is about 100 nucleotides to about 500,000 nucleotides in length. In some instances, the region of interest is about 100 nucleotides to about 300,000 base pairs in length. In some instances, the region of interest is about 1000 nucleotides to about 1,000,000 nucleotides in length. In some instances, the region of interest is about 1000 nucleotides to about 500,000 nucleotides in length. In some instances, the region of interest is about 1000 nucleotides to about 300,000 nucleotides in length. In some instances, the region of interest is about 10,000 nucleotides to about 1,000,000 nucleotides in length. In some instances, the region of interest is about 10,000 nucleotides to about 500,000 nucleotides in length. In some instances, the region of interest is about 10,000 nucleotides to about 300,000 nucleotides in length. In some instances, the region of interest is about 300,000 nucleotides in length.

In some instances, the sequence corresponding to the region of interest is at least about 5 nucleotides in length. In some instances, the sequence corresponding to the region of interest is at least about 8 nucleotides in length. In some instances, the sequence corresponding to the region of interest is at least about 10 nucleotides in length. In some instances, the sequence corresponding to the region of interest is at least about 15 nucleotides in length. In some instances, the sequence corresponding to the region of interest is at least about 20 nucleotides in length. In some instances, the sequence corresponding to the region of interest is at least about 50 nucleotides in length. In some instances, the sequence corresponding to the region of interest is at least about 100 nucleotides in length. In some instances, the sequence is about 5 nucleotides to about 1000 nucleotides in length. In some instances, the sequence is about 10 nucleotides to about 1000 nucleotides in length. In some instances, the sequence is about 10 nucleotides to about 500 nucleotides in length. In some instances, the sequence is about 10 nucleotides to about 400 nucleotides in length. In some instances, the sequence is about 10 nucleotides to about 300 nucleotides in length. In some instances, the sequence is about 50 nucleotides to about 1000 nucleotides in length. In some instances, the sequence is about 50 nucleotides to about 500 nucleotides in length.

In some instances, devices, systems and kits disclosed herein comprise at least one of an oligonucleotide probe and oligonucleotide primer that is capable of annealing to a strand of a cell-free nucleic acid, wherein the cell-free nucleic acid comprises a sequence corresponding to a sub-region of interest disclosed herein. In some instances, the sub-region is represented by a sequence that is present in the region of interest more than once. In some instances, the sub-region is about 10 to about 1000 nucleotides in length. In some instances, the sub-region is about 50 to about 500 nucleotides in length. In some instances, the sub-region is about 50 to about 250 nucleotides in length. In some instances, the sub-region is about 50 to about 150 nucleotides in length. In some instances, the sub-region is about 100 nucleotides in length.

In some instances, devices, systems and kits disclosed herein comprise at least one oligonucleotide primer, wherein the oligonucleotide primer has a sequence complementary to or corresponding to a Y chromosome sequence. In some instances, devices, systems and kits disclosed herein comprise a pair of oligonucleotide primers, wherein the pair of oligonucleotide primers have sequences complementary to or corresponding to a Y chromosome sequence. In some instances, devices, systems and kits disclosed herein comprise at least one oligonucleotide primer, wherein the oligonucleotide primer comprises a sequence complementary to or corresponding to a Y chromosome sequence. In some instances, devices, systems and kits disclosed herein comprise a pair of oligonucleotide primers, wherein the pair of oligonucleotide primers comprise sequences complementary to or corresponding to a Y chromosome sequence. In some instances, devices, systems and kits disclosed herein comprise at least one oligonucleotide primer, wherein the oligonucleotide primer consists of a sequence complementary to or corresponding to a Y chromosome sequence. In some instances, devices, systems and kits disclosed herein comprise a pair of oligonucleotide primers, wherein the pair of oligonucleotide primers consists of sequences complementary to or corresponding to a Y chromosome sequence. In some instances, the sequence(s) complementary to or corresponding to a Y chromosome sequence is at least 75% identical to a wild-type human Y chromosome sequence. In some instances, the sequence(s) complementary to or corresponding to a Y chromosome sequence is at least 80% identical to a wild-type human Y chromosome sequence. In some instances, the sequence(s) complementary to or corresponding to a Y chromosome sequence is at least 85% identical to a wild-type human Y chromosome sequence. In some instances, the sequence(s) complementary to or corresponding to a Y chromosome sequence is at least 80% identical to a wild-type human Y chromosome sequence. In some instances, the sequence(s) complementary to or corresponding to a Y chromosome sequence is at least 90% identical to a wild-type human Y chromosome sequence. In some instances, the sequence(s) complementary to or corresponding to a Y chromosome sequence is at least 95% identical to a wild-type human Y chromosome sequence. In some instances, the sequence(s) complementary to or corresponding to a Y chromosome sequence is at least 97% identical to a wild-type human Y chromosome sequence. In some instances, the sequence(s) complementary to or corresponding to a Y chromosome sequence is 100% identical to a wild-type human Y chromosome sequence.

In some instances, devices, systems and kits disclosed herein comprise at least one of an oligonucleotide probe and oligonucleotide primer that is capable of annealing to a strand of a cell-free nucleic acid, wherein the cell-free nucleic acid comprises a sequence corresponding to a Y chromosome region, or portion thereof, wherein the portion thereof has a given length. In some instances, the length of the portion thereof is about 10 nucleotides to about 100 nucleotides. In some instances, the length of the portion thereof is about 100 nucleotides to about 1000 nucleotides. In some instances, the length of the portion thereof is about 1000 nucleotides to about 10,000 nucleotides. In some instances, the length of the portion thereof is about 10,000 nucleotides to about 100,000 nucleotides.

In some instances, the region of interest is a Y chromosome region, or portion thereof, that comprises a sequence that is present on the Y chromosome more than once. In some instances, the Y chromosome region is located between position 20000000 and position 21000000 of the Y chromosome. In some instances, the Y chromosome region is located between position 20500000 and position 21000000 of the Y chromosome. In some instances, the Y chromosome region is located between position 20000000 and position 20500000 of the Y chromosome. In some instances, the Y chromosome region is located between position 20000000 and position 20250000 of the Y chromosome. In some instances, the Y chromosome region is located between position 20250000 and position 20500000 of the Y chromosome. In some instances, the Y chromosome region is located between position 20500000 and position 20750000 of the Y chromosome. In some instances, the Y chromosome region is located between position 20750000 and position 21000000 of the Y chromosome. In some instances, the Y chromosome region is located between position 20080000 and position 20400000 of the Y chromosome. In some instances, the Y chromosome region is located between position 20082000 and position 20351000 of the Y chromosome. In some instances, the Y chromosome region is located between position 20082183 and position 20350897 of the Y chromosome.

In some instances, devices, systems and kits disclosed herein comprise at least one of an oligonucleotide probe and oligonucleotide primer that is capable of annealing to a strand of a cell-free nucleic acid, wherein the cell free nucleic acid comprises a sequence corresponding to a Y chromosome sub-region. In some instances, corresponding is 100% identical. In some instances, corresponding is at least 99% identical. In some instances, corresponding is at least 98% identical. In some instances, corresponding is at least 95% identical. In some instances, corresponding is at least 90% identical.

In some instances, devices, systems and kits disclosed herein comprise at least one of an oligonucleotide probe and oligonucleotide primer that is capable of annealing to a strand of a cell-free nucleic acid, wherein the cell free nucleic acid comprises a sequence corresponding to a Y chromosome sub-region between start position 20350799 and end position 20350897 of the Y chromosome. In some instances, the sequence corresponds to at least 10 nucleotides of a Y chromosome sub-region between start position 20350799 and end position 20350897 of the Y chromosome. In some instances, the sequence corresponds to at least 50 nucleotides of a Y chromosome sub-region between start position 20350799 and end position 20350897 of the Y chromosome. In some instances, the sequence corresponds to at least about 10 to at least about 1000 nucleotides of a Y chromosome sub-region between start position 20350799 and end position 20350897 of the Y chromosome. In some instances, the sequence corresponds to at least about 50 to at least about 500 nucleotides of a Y chromosome sub-region between start position 20350799 and end position 20350897 of the Y chromosome. In some instances, the sequence corresponds to at least about 50 to at least about 150 nucleotides of a Y chromosome sub-region between start position 20350799 and end position 20350897 of the Y chromosome.

In some instances, devices, systems and kits disclosed herein comprise at least one of an oligonucleotide probe and oligonucleotide primer that is capable of annealing to a strand of a cell-free nucleic acid, wherein the cell free nucleic acid comprises a sequence corresponding to a Y chromosome sub-region between start position 20349236 and end position 20349318 of the Y chromosome. In some instances, the sequence corresponds to at least 10 nucleotides of a Y chromosome sub-region between start position 20349236 and end position 20349318 of the Y chromosome. In some instances, the sequence corresponds to at least 50 nucleotides of a Y chromosome sub-region between start position 20349236 and end position 20349318 of the Y chromosome. In some instances, the sequence corresponds to at least about 10 to at least about 1000 nucleotides of a Y chromosome sub-region between start position 20349236 and end position 20349318 of the Y chromosome. In some instances, the sequence corresponds to at least about 50 to at least about 500 nucleotides of a Y chromosome sub-region between start position 20349236 and end position 20349318 of the Y chromosome. In some instances, the sequence corresponds to at least about 50 to at least about 150 nucleotides of a Y chromosome sub-region between start position 20349236 and end position 20349318 of the Y chromosome.

In some instances, devices, systems and kits disclosed herein comprise at least one of an oligonucleotide probe and oligonucleotide primer that is capable of annealing to a strand of a cell-free nucleic acid, wherein the cell free nucleic acid comprises a sequence corresponding to a Y chromosome sub-region between start position 20350231 and end position 20350323 of the Y chromosome. In some instances, the sequence corresponds to at least 10 nucleotides of a Y chromosome sub-region between start position 20350231 and end position 20350323 of the Y chromosome. In some instances, the sequence corresponds to at least 50 nucleotides of a Y chromosome sub-region between start position 20350231 and end position 20350323 of the Y chromosome. In some instances, the sequence corresponds to at least about 10 to at least about 1000 nucleotides of a Y chromosome sub-region between start position 20350231 and end position 20350323 of the Y chromosome. In some instances, the sequence corresponds to at least about 50 to at least about 500 nucleotides of a Y chromosome sub-region between start position 20350231 and end position 20350323 of the Y chromosome. In some instances, the sequence corresponds to at least about 50 to at least about 150 nucleotides of a Y chromosome sub-region between start position 20350231 and end position 20350323 of the Y chromosome.

In some instances, devices, systems and kits disclosed herein comprise at least one of an oligonucleotide probe and oligonucleotide primer that is capable of annealing to a strand of a cell-free nucleic acid, wherein the cell free nucleic acid comprises a sequence corresponding to a Y chromosome sub-region between start position 20350601 and end position 20350699 of the Y chromosome. In some instances, the sequence corresponds to at least 10 nucleotides of a Y chromosome sub-region between start position 20350601 and end position 20350699 of the Y chromosome. In some instances, the sequence corresponds to at least 50 nucleotides of a Y chromosome sub-region between start position 20350601 and end position 20350699 of the Y chromosome. In some instances, the sequence corresponds to at least about 10 to at least about 1000 nucleotides of a Y chromosome sub-region between start position 20350601 and end position 20350699 of the Y chromosome. In some instances, the sequence corresponds to at least about 50 to at least about 500 nucleotides of a Y chromosome sub-region between start position 20350601 and end position 20350699 of the Y chromosome. In some instances, the sequence corresponds to at least about 50 to at least about 150 nucleotides of a Y chromosome sub-region between start position 20350601 and end position 20350699 of the Y chromosome.

In some instances, devices, systems and kits disclosed herein comprise at least one of an oligonucleotide probe and oligonucleotide primer that is capable of annealing to a strand of a cell-free nucleic acid, wherein the cell free nucleic acid comprises a sequence corresponding to a Y chromosome sub-region between start position 20082183 and end position 20082281 of the Y chromosome. In some instances, the sequence corresponds to at least 10 nucleotides of a Y chromosome sub-region between start position 20082183 and end position 20082281 of the Y chromosome. In some instances, the sequence corresponds to at least 50 nucleotides of a Y chromosome sub-region between start position 20082183 and end position 20082281 of the Y chromosome. In some instances, the sequence corresponds to at least about 10 to at least about 1000 nucleotides of a Y chromosome sub-region between start position 20082183 and end position 20082281 of the Y chromosome. In some instances, the sequence corresponds to at least about 50 to at least about 500 nucleotides of a Y chromosome sub-region between start position 20082183 and end position 20082281 of the Y chromosome. In some instances, the sequence corresponds to at least about 50 to at least about 150 nucleotides of a Y chromosome sub-region between start position 20082183 and end position 20082281 of the Y chromosome.

In some instances, devices, systems and kits disclosed herein comprise at least one of an oligonucleotide probe and oligonucleotide primer that is capable of annealing to a strand of a cell-free nucleic acid, wherein the cell free nucleic acid comprises a sequence corresponding to a Y chromosome sub-region between start position 56673250 and end position 56771489 of the Y chromosome. In some instances, the sequence corresponds to at least 10 nucleotides of a Y chromosome sub-region between start position 56673250 and end position 56771489 of the Y chromosome. In some instances, the sequence corresponds to at least 50 nucleotides of a Y chromosome sub-region between start position 56673250 and end position 56771489 of the Y chromosome. In some instances, the sequence corresponds to at least about 10 to at least about 1000 nucleotides of a Y chromosome sub-region between start position 56673250 and end position 56771489 of the Y chromosome. In some instances, the sequence corresponds to at least about 50 to at least about 500 nucleotides of a Y chromosome sub-region between start position 56673250 and end position 56771489 of the Y chromosome. In some instances, the sequence corresponds to at least about 50 to at least about 150 nucleotides of a Y chromosome sub-region between start position 56673250 and end position 56771489 of the Y chromosome. In some instances, devices, systems and kits disclosed herein comprise at least one of an oligonucleotide probe and oligonucleotide primer that is capable of annealing to a strand of a cell-free nucleic acid, wherein the cell-free nucleic acid comprises a sequence corresponding to a Y chromosome sub-region, wherein the sequence is selected from SEQ ID NOS.: 1-5, shown in Table 1. In some instances, the sequence is at least 60% identical to a sequence selected from SEQ ID NOS.: 1-5. In some instances, the sequence is at least 65% identical to a sequence selected from SEQ ID NOS.: 1-5. In some instances, the sequence is at least 70% identical to a sequence selected from SEQ ID NOS.: 1-5. In some instances, the sequence is at least 75% identical to a sequence selected from SEQ ID NOS.: 1-5. In some instances, the sequence is at least 80% identical to a sequence selected from SEQ ID NOS.: 1-5. In some instances, the sequence is at least 85% identical to a sequence selected from SEQ ID NOS.: 1-5. In some instances, the sequence is at least 90% identical to a sequence selected from SEQ ID NOS.: 1-5. In some instances, the sequence is at least 95% identical to a sequence selected from SEQ ID NOS.: 1-5. In some instances, the sequence is at least 98% identical to a sequence selected from SEQ ID NOS.: 1-5. In some instances, the sequence is at least 99% identical to a sequence selected from SEQ ID NOS.: 1-5. In some instances, corresponding is 100% identical. In some instances, the sequence comprises at least 10 consecutive nucleotides of SEQ ID NOS.: 1-5. In some instances, the sequence comprises at least 15 consecutive nucleotides of SEQ ID NOS.: 1-5. In some instances, the sequence comprises at least 20 consecutive nucleotides of SEQ ID NOS.: 1-5. In some instances, the sequence comprises at least 25 consecutive nucleotides of SEQ ID NOS.: 1-5. In some instances, the sequence comprises at least 50 consecutive nucleotides of SEQ ID NOS.: 1-5.

TABLE 1
Sequences of Y chromosome sub-regions

| | |
|---|---|
| SEQ ID | TTACAGCAGTTAAAGGTGTTATGTCCAGAGTTTGTTTCT |
| NO. 1 | GCAGATGTGTCCAGAGTTTCTTCCTTCTGGCAGGTTCAT |
| | GGTCTTTCTCACTTCAAGAATGA |
| SEQ ID | TTCTGGCAGGTTCATGGTCTTGCTCACTTCAAGAATGAA |
| NO. 2 | GCTGCAGACTTTTGTGGTGAGTGTTACAGCAGTTAAAGT |
| | TGTTATGTC |
| SEQ ID | TCTTCCTTCTGGCAGGTTCATGGTCTTGCTCACTTCACT |
| NO. 3 | AATGAAGGTGCAGACCTTACTGGTGAGTGTTACAGCACT |
| | TAAAGGTGTTATGTCC |
| SEQ ID | AGTTTCTTCCTTCTGGCAGGTTCATGGTCTTGTTCACTT |
| NO. 4 | CAAGAATGAAGCTGCAGACCTTAGTGGTGAGTGTTACAG |
| | CACTTAAAGGTGTTATGTCCAGAGTT |
| SEQ ID | TAACACCTTTAAGTGCTGTAACACTCACCACTAAATTCT |
| NO. 5 | GCAGCTTCACTCTTGAAGTGAGCAAGACCATGAACCTGC |
| | CAGAAGGAAGAAACTCTGAACACATCTG |

In some instances, devices, systems and kits disclosed herein comprise at least one of an oligonucleotide probe and oligonucleotide primer that is capable of annealing to a strand of a cell-free nucleic acid, wherein the cell-free nucleic acid comprises a sequence corresponding to a Y chromosome sub-region, wherein the sequence is selected from SEQ ID NOS.: 30-34, shown in Table 3. In some instances, the sequence is at least 60% identical to a sequence selected from SEQ ID NOS.: 30-34. In some instances, the sequence is at least 65% identical to a sequence selected from SEQ ID NOS.: 30-34. In some instances, the sequence is at least 70% identical to a sequence selected from SEQ ID NOS.: 30-34. In some instances, the sequence is at least 75% identical to a sequence selected from SEQ ID NOS.: 30-34. In some instances, the sequence is at least 80% identical to a sequence selected from SEQ ID NOS.: 30-34. In some instances, the sequence is at least 85% identical to a sequence selected from SEQ ID NOS.: 30-34. In some instances, the sequence is at least 90% identical to a sequence selected from SEQ ID NOS.: 30-34. In some instances, the sequence is at least 95% identical to a sequence selected from SEQ ID NOS.: 30-34. In some instances, the sequence is at least 98% identical to a sequence selected from SEQ ID NOS.: 30-34. In some instances, the sequence is at least 99% identical to a sequence selected from SEQ ID NOS.: 30-34. In some instances, corresponding is 100% identical. In some instances, the sequence comprises at least 10 consecutive nucleotides of SEQ ID NOS.: 30-34. In some instances, the sequence comprises at least 15 consecutive nucleotides of SEQ ID NOS.: 30-34. In some instances, the sequence comprises at least 20 consecutive nucleotides of SEQ ID NOS.: 30-34. In some instances, the sequence comprises at least 25 consecutive nucleotides of SEQ ID NOS.: 30-34. In some instances, the sequence comprises at least 50 consecutive nucleotides of SEQ ID NOS.: 30-34. Example 3 describes results of assays that analyze Y chromosome sub-regions having sequences selected from SEQ ID NOS.: 30-34.

In some instances, devices, systems and kits disclosed herein comprise at least one of an oligonucleotide probe and oligonucleotide primer that is capable of annealing to a strand of a cell-free nucleic acid, wherein the cell-free nucleic acid comprises a sequence between chrY:56672250 and chrY:56772489, (according to Genome Build 38). In some instances, devices, systems and kits disclosed herein comprise at least one of an oligonucleotide probe and oligonucleotide primer that is capable of annealing to a strand of a cell-free nucleic acid, wherein the cell-free nucleic acid comprises a sequence between chrY:56673250 and chrY:56771489 (according to Genome Build 38). Example 4 presents results using a pair of primers that amplify such sequences (shown in Table 5). The pair of primers may be selected from primers represented by two sequences selected from SEQ ID NOS.: 37 and 38, 39 and 40, 41 and 42, 43 and 44, 45 and 46, 47 and 48, 49 and 50, 51 and 52, 53 and 54, 55 and 56, 57 and 58, 59 and 60, 61 and 62, 63 and 64, 65 and 66, 67 and 68, 69 and 70, 71 and 72, 73 and 74, 75 and 76, 77 and 78, 79 and 80, 81 and 82, 83 and 84, 85 and 86, 87 and 88, 89 and 90, 91 and 92, 93 and 94, 95 and 96, 97 and 98, 99 and 100, 101 and 102, 103 and 104, 105 and 106, 107 and 108, 109 and 110, 111 and 112, 113 and 114, 115 and 116, 117 and 118, 119 and 120, 121 and 122, 123 and 124, 125 and 126, 127 and 128, 129 and 130, 131 and 132, 133 and 134, 135 and 136, 137 and 138, and 139 and 140. In some instances, the pair of primers are represented by a sequence that is at least 80% identical to a sense primer in Table 5 and at least 90% identical to an antisense primer in Table 5. In some instances, the pair of primers are represented by a sequence that is at least 90% identical to a sense primer in Table 5 and at least 90% identical to an antisense primer in Table 5.

In some instances, devices, systems and kits disclosed herein comprise at least one of an oligonucleotide probe and oligonucleotide primer that is capable of annealing to a strand of a cell-free nucleic acid, wherein the cell-free nucleic acid comprises a sequence corresponding to a Y chromosome sub-region, wherein the sequence is selected from SEQ ID NOS.: 141-192, shown in Table 5. In some instances, the sequence is at least 60% identical to a sequence selected from SEQ ID NOS.: 141-192. In some instances, the sequence is at least 65% identical to a sequence selected from SEQ ID NOS.: 141-192. In some instances, the sequence is at least 70% identical to a sequence selected from SEQ ID NOS.: 141-192. In some instances, the sequence is at least 75% identical to a sequence selected from SEQ ID NOS.: 141-192. In some instances, the sequence is at least 80% identical to a sequence selected from SEQ ID NOS.: 141-192. In some instances, the sequence is at least 85% identical to a sequence selected from SEQ ID NOS.: 141-192. In some instances, the sequence is at least 90% identical to a sequence selected from SEQ ID NOS.: 141-192. In some instances, the sequence is at least 95% identical to a sequence selected from SEQ ID NOS.: 141-192. In some instances, the sequence is at least 98% identical to a sequence selected from SEQ ID NOS.: 141-192. In some instances, the sequence is at least 99% identical to a sequence selected from SEQ ID NOS.: 141-192. In some instances, corresponding is 100% identical. In some instances, the sequence comprises at least 10 consecutive nucleotides of SEQ ID NOS.: 141-192. In some instances, the sequence comprises at least 15 consecutive nucleotides of SEQ ID NOS.: 141-192. In some instances, the sequence comprises at least 20 consecutive nucleotides of SEQ ID NOS.: 141-192. In some instances, the sequence comprises at least 25 consecutive nucleotides of SEQ ID NOS.: 141-192. In some instances, the sequence comprises at least 50 consecutive nucleotides of SEQ ID NOS.: 141-192. Example 4 describes results of assays that analyze Y chromosome sub-regions having sequences selected from SEQ ID NOS.: 141-192.

### Nucleic Acid Sequencing

In some instances, devices, systems and kits disclosed herein comprise a nucleic acid sequencer. In some instances, devices, systems and kits disclosed herein are configured to amplify nucleic acids and sequence the resulting amplified nucleic acids. In some instances, devices, systems and kits disclosed herein are configured to sequence nucleic acids without amplifying nucleic acids. In some instances, devices, systems and kits disclosed herein comprise a nucleic acid sequencer, but do not comprise a nucleic acid amplifying reagent or nucleic acid amplifying component. In some instances, the nucleic acid sequencer comprises a signal detector that detects a signal that reflects successful amplification or unsuccessful amplification. In some instances, the nucleic acid sequencer is the signal detector. In some instances, the signal detector comprises the nucleic acid sequencer.

In some instances, the nucleic acid sequencer has a communication connection with an electronic device that analyzes sequencing reads from the nucleic acid sequencer. In some instances the communication connection is hard wired. In some instances the communication connection is wireless. For example, a cell phone app or computer software, such as those disclosed herein, may receive the sequencing reads, and based on the sequencing reads, display or report genetic information about the sample (e.g., presence of a disease/infection, response to a drug, gender of a fetus).

In some instances, the nucleic acid sequencer comprises a nanopore sequencer. In some instances, the nanopore sequencer comprises a nanopore. In some instances, the nanopore sequencer comprises a membrane and solutions that create a current across the membrane and drive movement of charged molecules (e.g., nucleic acids) through the nanopore. In some instances, the nanopore sequencer comprises a transmembrane protein, a portion thereof, or a modification thereof. In some instances, the transmembrane protein is a bacterial protein. In some instances, the transmembrane protein is not a bacterial protein. In some instances, the nanopore is synthetic. In some instances, the nanopore performs solid state nanopore sequencing. In some instances, the nanopore sequencer is described as pocket-sized, portable, or roughly the size of a cell phone. In some instances, the nanopore sequencer is configured to sequence at least one of RNA and DNA. Non-limiting examples of nanopore sequencing devices include Oxford Nanopore Technologies MinION and SmidgION nanopore sequencing USB devices. Both of these devices are small enough to be handheld. Nanopore sequencing devices and components are further described in reviews by Howorka (Nat Nanotechnol. 2017 Jul. 6; 12(7):619-630), and Garrido-Cardenas et al. (Sensors (Basel). 2017 Mar. 14; 17(3)), both incorporated herein by reference. Other non-limiting examples of nanopore sequencing devices are offered by Electronic Biosciences, Two Pore Guys, Stratos, and Agilent (technology originally from Genia).

In some instances, devices, systems and kits disclosed herein comprise reagents and components required for bisulfite sequencing to detect epigenetic modifications. For instance, a long region with many methylation markers can be fragmented. Here, each fragment carrying a methylation marker can be an independent signal. Signals from all the fragments are sufficient in combination to obtain useful genetic information.

### Capture and Detection

In some instances, devices, systems and kits disclosed herein comprise at least one of a capture component, signal detector, and a detection reagent for detecting a nucleic acid in the biological sample. In some instances, the capture component and the signal detector are integrated. In some instances, the capture component comprises a solid support. In some instances the solid support comprises a bead, a chip, a strip, a membrane, a matrix, a column a plate, or a combination thereof. In some instances, the capture component comprises a binding moiety disclosed herein.

In some instances, devices, systems and kits disclosed herein comprise at least one probe for a nucleic acid having a sequence of interest. In some instances, the sequence of interest is specific to a Y chromosome. In some instances, devices, systems and kits disclosed herein comprise at least one probe for a paternally inherited sequence that is not present in the maternal DNA. In some instances, devices, systems and kits disclosed herein comprise at least one probe for a paternally inherited single nucleotide polymorphism. In some instances, devices, systems and kits disclosed herein comprise at least one probe for an epigenetically modified region of a chromosome or fragment thereof. In some instances, the epigenetic modification of the epigenetically modified region of a chromosome is indicative of gender or a marker of gender. In some instances, the chromosome is a Y chromosome. In some instances, the chromosome is an X chromosome. In some instances, the chromosome is an autosome. In some instances, the probe comprises a peptide, an antibody, an antigen binding antibody fragment, a nucleic acid or a small molecule.

In some instances, the capture component comprises a binding moiety described herein. In some instances, the binding moiety is present in a lateral flow assay. In some instances, the binding moiety is added to the sample before the sample is added to the lateral flow assay. In some instances, the binding moiety comprises a signaling molecule. In some instances, the binding moiety is physically associated with a signaling molecule. In some instances, the binding moiety is capable of physically associating with a signaling molecule. In some instances, the binding moiety is connected to a signaling molecule. Non-limiting examples of signaling molecules include a gold particle, a fluorescent particle, a luminescent particle, and a dye molecule. In some instances the capture component comprises a binding moiety that is capable of interacting with an amplification product described herein. In some instances the capture component comprises a binding moiety that is capable of interacting with a tag on an amplification product described herein.

In some instances, devices, systems and kits disclosed herein comprise a detection system. In some instances, the detection system comprises a signal detector. Non-limiting examples of a signal detector include a fluorescence reader, a colorimeter, a sensor, a wire, a circuit, a receiver. In some instances, the detection system comprises a detection reagent. Non-limiting examples of a detection reagent include a fluorophore, a chemical, a nanoparticle, an antibody, and a nucleic acid probe. In some instances, the detection system comprises a pH sensor and a complementary metal-oxide semiconductor, which can be used to detect changes in pH. In some instances, production of an amplification product by devices, systems, kits or methods disclosed herein changes the pH, thereby indicating gender.

In some instances, the detection system comprises a signal detector. In some instances, the signal detector is a photodetector that detects photons. In some instances, the signal detector detects fluorescence. In some instances, the signal detector detects a chemical or compound. In some instances, the signal detector detects a chemical that is released when the amplification product is produced. In some instances, the signal detector detects a chemical that is released when the amplification product is added to the detection system. In some instances, the signal detector detects a compound that is produced when the amplification product is produced. In some instances, the signal detector detects a compound that is produced when the amplification product is added to the detection system.

In some instances, the signal detector detects an electrical signal. In some instances, the signal detector comprises an electrode. In some instances, the signal detector comprises a circuit a current, or a current generator. In some instances, the circuit or current is provided by a gradient of two or more solutions or polymers. In some instances, the circuit or current is provided by an energy source (e.g., battery, wire from electrical outlet). In some instances, nucleic acids, amplification products, chemicals or compounds disclosed herein provide an electrical signal by disrupting the current and the signal detector detects the electrical signal. In some instances, the signal detector detects light. In some instances, the signal detector comprises a light sensor. In some instances, the signal detector comprises a camera. In some instances, the signal detector comprises a cell phone camera or a component thereof.

In some instances, the signal detector comprises a nanowire that detects the charge of different bases in nucleic acids. In some instances, the nanowire has a diameter of about 1 nm to about 99 nm. In some instances, the nanowire has a diameter of about 1 nm to about 999 nm. In some instances, the nanowire comprises an inorganic molecule, e.g., nickel, platinum, silicon, gold, zinc, graphene, or titanium. In some instances, the nanowire comprises an organic molecule (e.g., a nucleotide).

In some instances, the detection system comprises an assay assembly, wherein the assay assembly is capable of detecting a target analyte (e.g., nucleic acid amplification product). In some instances, the assay assembly comprises a lateral flow strip, also referred to herein and in the field, as a lateral flow assay, lateral flow test or lateral flow device. In some instances, a lateral flow assay provides a fast, inexpensive, and technically simple method to detect amplification products disclosed herein. Generally, lateral flow assays disclosed herein comprise a porous material or porous matrix that transports a fluid, and a detector that detects the amplification product when it is present. The porous material may comprise a porous paper, a polymer structure, a sintered polymer, or a combination thereof. In some instances, the lateral flow assay transports the biological fluid or portion thereof (e.g., plasma of blood sample). In some instances, the lateral flow assay transports a solution containing the biological fluid or portion thereof. For instance, methods may comprise adding a solution to the biological fluid before or during addition of the sample to the device or system. The solution may comprise a salt, a polymer, or any other component that facilitates transport of the sample and or amplification product through the lateral flow assay. In some instances, nucleic acids are amplified after they have traveled through the lateral flow strip.

In some instances, devices, the detection system comprises a lateral flow device, wherein the lateral flow device comprises multiple sectors or zones, wherein each desired function can be present in a separate sector or zone. In general, in a lateral flow device, a liquid sample, e.g., a body fluid sample as described herein, containing the target analyte moves with or without the assistance of external forces through sectors or zones of the lateral flow device. In some instances, the target analyte moves without the assistance of external forces, e.g., by capillary action. In some instances, the target analyte moves with assistance of external forces, e.g., by facilitation of capillary action by movement of the lateral flow device. Movement can comprise any motion caused by external input, e.g., shaking, turning, centrifuging, applying an electrical field or magnetic field, applying an active pump, applying a vacuum, or rocking of the lateral flow device.

In some instances, the lateral flow device is a lateral flow test strip, comprising zones or sectors that are situated laterally, e.g., behind or ahead of each other. In general, a lateral flow test strip allows accessibility of the functional zones or sectors from each side of (e.g., above and below) the test strip as a result of exposure of a large surface area of each functional zone or sector. This facilitates the addition of reagents, including those used in sample purification, or target analyte amplification, detection, and/or determination.

Any suitable lateral flow test strip detection format known to those of skill in the art is contemplated for use in an assay assembly of the methods, devices, systems and kits disclosed herein. Lateral flow test strip detection formats are well known and have been described in the literature. Lateral flow test strip assay formats are generally described by, e.g., Sharma et al., (2015) Biosensors 5:577-601, incorporated by reference herein in its entirety. Detection of nucleic acids using lateral flow test strip sandwich assay formats is described by, e.g., U.S. Pat. No. 9,121,849, “Lateral Flow Assays,” incorporated by reference herein in its entirety. Detection of nucleic acids using lateral flow test strip competitive assay formats is described by, e.g., U.S. Pat. No. 9,423,399, “Lateral Flow Assays for Tagged Analytes,” incorporated by reference herein in its entirety.

In some instances, a lateral flow test strip detects the target analyte in a test sample using a sandwich format, a competitive format, or a multiplex detection format. In a traditional sandwich assay format, the detected signal is directly proportional to the amount of the target analyte present in the sample, so that increasing amounts of the target analyte lead to increasing signal intensity. In traditional competitive assay formats, the detected signal has an inverse relationship with the amount of analyte present, and increasing amounts of analyte lead to decreasing signal intensity.

In a lateral flow sandwich format, the test sample typically is applied to a sample application pad at one end of a test strip. The applied test sample flows through the test strip, from the sample application pad to a conjugate pad located adjacent to the sample application pad, where the conjugate pad is downstream in the direction of sample flow. In some instances, the conjugate pad comprises a labeled, reversibly-immobilized probe, e.g., an antibody or aptamer labeled with, e.g., a dye, enzyme, or nanoparticle. A labeled probe-target analyte complex is formed if the target analyte is present in the test sample. This complex then flows to a first test zone or sector (e.g., a test line) comprising an immobilized second probe which is specific to the target analyte, thereby trapping any labeled probe-target analyte complex. In some instances, the intensity or magnitude of signal, e.g., color, at the first test zone or sector is used to indicate the presence or absence, quantity, or presence and quantity of target analyte in the test sample. A second test zone or sector can comprise a third probe that binds to excess labeled probe. If the applied test sample comprises the target analyte, little or no excess labeled probe will be present on the test strip following capture of the target analyte by the labeled probe on the conjugate pad. Consequently, the second test zone or sector will not bind any labeled probe, and little or no signal (e.g., color) at the second test zone or sector is expected to be observed. The absence of signal at the second test zone or sector thus can provide assurance that signal observed in the first test zone or sector is due to the presence of the target analyte.

In some instances, devices and systems disclosed herein comprise a sandwich assay. In some instances, the sandwich assay is configured to receive a biological sample disclosed herein and retain sample components (e.g., nucleic acids, cells, microparticles). In some instances, the sandwich assay is configured to receive a flow solution that flushes non-nucleic acid components of the biological sample (e.g., proteins, cells, microparticles), leaving nucleic acids of the biological sample behind. In some instances, the sandwich assay comprises a membrane that binds nucleic acids to help retain the nucleic acids when the flow solution is applied. Non-limiting examples of a membrane the binds nucleic acids includes chitosan modified nitrocellulose.

Similarly, in a lateral flow competitive format a test sample is applied to a sample application pad at one end of a test strip, and the target analyte binds to a labeled probe to form a probe-target analyte complex in a conjugate pad downstream of the sample application pad. In the competitive format, the first test zone or sector typically comprises the target analyte or an analog of the target analyte. The target analyte in the first test zone or sector binds any free labeled probe that did not bind to the test analyte in the conjugate pad. Thus, the amount of signal observed in the first test zone or sector is higher when there is no target analyte in the applied test sample than when target analyte is present. A second test zone or sector comprises a probe that specifically binds to the probe-target analyte complex. The amount of signal observed in this second test zone or sector is higher when the target analyte is present in the applied test sample.

In a lateral flow test strip multiplex detection format, more than one target analyte is detected using the test strip through the use of additional test zones or sectors comprising, e.g., probes specific for each of the target analytes.

In some instances, the lateral flow device is a layered lateral flow device, comprising zones or sectors that are present in layers situated medially, e.g., above or below each other. In some instances, one or more zones or sectors are present in a given layer. In some instances, each zone or sector is present in an individual layer. In some instances, a layer comprises multiple zones or sectors. In some instances, the layers are laminated. In a layered lateral flow device, processes controlled by diffusion and directed by the concentration gradient are possible driving forces. For example, multilayer analytical elements for fluorometric assay or fluorometric quantitative analysis of an analyte contained in a sample liquid are described in EP0097952, “Multilayer analytical element,” incorporated by reference herein.

A lateral flow device can comprise one or more functional zones or sectors. In some instances, the test assembly comprises 1 to 20 functional zones or sectors. In some instances, the functional zones ore sectors comprise at least one sample purification zone or sector, at least one target analyte amplification zone or sector, at least one target analyte detection zone or sector, and at least one target analyte determination zone or sector.

In some instances, the target analyte is a nucleic acid sequence, and the lateral flow device is a nucleic acid lateral flow assay. In some instances, devices, systems and kits disclosed herein comprise a nucleic acid lateral flow assay, wherein the nucleic acid lateral flow assay comprises nucleic acid amplification function. In some instances, target nucleic acid amplification that is carried out by the nucleic acid amplification function takes place prior to, or at the same time as, detection of the amplified nucleic acid species. In some instances, detection comprises one or more of qualitative, semi-quantitative, or quantitative determination of the presence of the target analyte.

In some instances, the devices, systems and kits disclosed herein comprise an assay assembly wherein a target nucleic acid analyte is amplified in a lateral flow test strip to generate labeled amplification products, or amplification products that can be labeled after amplification. In some instances, a label is present on one or more amplification primers, or subsequently conjugated to one or more amplification primers, following amplification. In some instances, at least one target nucleic acid amplification product is detected on the lateral flow test strip. For example, one or more zones or sectors on the lateral flow test strip may comprise a probe that is specific for a target nucleic acid amplification product.

In some instances, the devices, systems and kits disclosed herein comprise a detector, wherein the detector comprises a graphene biosensor. Graphene biosensors are described, e.g., by Afsahi et al., in the article entitled, “Novel graphene-based biosensor for early detection of Zika virus infection, Biosensor and Bioelectronics,” (2018) 100:85-88.

In some instances, a detector disclosed herein comprises a nanopore, a nanosensor, or a nanoswitch. For instance, the detector may be capable of nanopore sequencing, a method of transporting a nucleic acid through a nanpore based on an electric current across a membrane, the detector measuring disruptions in the current corresponding to specific nucleotides. A nanoswitch or nanosensor undergoes a structural change upon exposure to the detectable signal. See, e.g., Koussa et al., “DNA nanoswitches: A quantitative platform for gel-based biomolecular interaction analysis,” (2015) Nature Methods, 12(2): 123-126

In some instances, the detector comprises a rapid multiplex biomarker assay where probes for an analyte of interest are produced on a chip that is used for real-time detection. Thus, there is no need for a tag, label or reporter. Binding of analytes to these probes causes a change in a refractive index that corresponds to a concentration of the analyte. All steps may be automated. Incubations may be not be necessary. Results may be available in less than an hour (e.g., 10-30 minutes). A non-limiting examples of such a detector is the Genalyte Maverick Detection System.

### Additional Tests

In some instances, devices, systems and kits disclosed herein comprise additional features, reagents, tests or assays for detection or analysis of biological components besides nucleic acids. By way of non-limiting example, the biological component may be selected from a protein, a peptide, a lipid, a fatty acid, a sterol, a carbohydrate, a viral component, a microbial component, and a combination thereof. These additional assays may be capable of detecting or analyzing biological components in the small volumes or sample sizes disclosed herein and throughout. An additional test may comprise a reagent capable of interacting with a biological component of interest. Non-limiting examples of such reagents include antibodies, peptides, oligonucleotides, aptamers, and small molecules, and combinations thereof. The reagent may comprise a detectable label. The reagent may be capable of interacting with a detectable label. The reagent may be capable of providing a detectable signal.

Additional tests may require one or more antibodies. For instance, the additional test may comprise reagents or components that provide for performing Immuno-PCR (IPCR). IPCR is a method wherein a first antibody for a protein of interest is immobilized and exposed to a sample. If the sample contains the protein of interest, it will be captured by the first antibody. The captured protein of interest is then exposed to a second antibody that binds the protein of interest. The second antibody has been coupled to a polynucleotide that can be detected by real-time PCR. Alternatively or additionally, the additional test may comprise reagents or components that provide for performing a proximity ligation assay (PLA), wherein the sample is exposed to two antibodies specific for a protein of interest, each antibody comprising an oligonucleotide. If both antibodies bind to the protein of interest, the oligonucleotides of each antibody will be close enough to be amplified and/or detected.

In some instances, devices, systems and kits disclosed herein comprise additional tests or assays beyond an assay for nucleic acids corresponding to the Y chromosome. In some instances, methods disclosed herein comprise testing a biological sample beyond testing for presence of a Y chromosome (gender test). In some instances, methods disclosed herein comprise characterizing a biological sample beyond testing for a presence of a Y chromosome. In some instances, devices, systems and kits disclosed herein comprise a test for a protein or peptide. In some instances, the protein is a hormone. In some instances, methods disclosed herein comprise testing, assaying or quantifying a protein. In some instances, devices, systems and kits disclosed herein comprise an assay for a presence or quantity of a nucleic acid and a presence or quantity of a protein or peptide. In some instances, the additional test is a test for gestational age. In some instances, the test for gestational age ensures the gender test is performed at a gestational age that is feasible for accurate gender detection. In some instances, the additional test is a pregnancy test. In some instances, the pregnancy test confirms that female is subject if a gender and/or gestational age are undetectable or undiscernible by a device, system or kit disclosed herein.

In some instances, devices, systems and kits disclosed herein comprise a pregnancy test for indicating, determining or verifying the female subject is pregnant. In some instances the pregnancy test comprises a reagent or component for measuring a pregnancy related factor. By way of non-limiting example, the pregnancy related factor may be human chorionic gonadotropin protein (hCG) and the reagent or component for hCG comprising an anti-hCG antibody. Also by way of non-limiting example, the pregnancy related factor may be an hCG transcript and the reagent or component for measuring the hCG transcript is an oligonucleotide probe or primer that hybridizes to the hCG transcript. In some instances, the pregnancy related factor is heat shock protein 10 kDa protein 1, also known as early-pregnancy factor (EPF).

In some instances, devices, systems and kits disclosed herein are capable of conveying the age of the fetus. For example, a signal may be generated from the device or system, wherein the level of the signal corresponds to the amount of hCG in the sample from the subject. This level or strength of the signal may be translated or equivocated with a numerical value representing the amount of hCG in the sample. The amount of hCG may indicate an approximate age of the fetus.

In some instances, devices, systems and kits disclosed herein provide an indication or verification of pregnancy, an indication or verification of gestational age, and an indication or verification of gender. In some instances, devices, systems and kits disclosed herein provide an indication of pregnancy, gestational age, and/or gender with at least about 90% confidence (e.g., 90% of the time, the indication is accurate). In some instances, devices, systems and kits disclosed herein provide an indication of pregnancy, gestational age, and/or gender with at least about 95% confidence. In some instances, devices, systems and kits disclosed herein provide an indication of pregnancy, gestational age, and/or gender with at least about 99% confidence.

### Performance Parameters

In some instances, the devices, systems and kits disclosed herein are operable at one or more temperatures. In some instances, the temperature of a component or reagent of the device system, or kit needs to be altered in order for the device system, or kit to be operable. Generally, devices, systems and kits are considered operable when they provide information (e.g., gender, infection, contamination) conveyed by biomarkers (e.g., RNA/DNA, peptides) in the biological sample. In some instances, temperature(s) at which the devices, systems, kits, components thereof, or reagents thereof are operable are obtained in a common household. By way of non-limiting example, temperature(s) obtained in a common household may be provided by room temperature, a refrigerator, a freezer, a microwave, a stove, an electric hot pot, hot/cold water bath, or an oven.

In some instances, devices, systems, kits, components thereof, or reagents thereof, as described herein, are operable at a single temperature. In some instances, devices, systems, kits, components thereof, or reagents thereof, as described herein, only require a single temperature to be operable. In some instances, devices, systems, kits, components thereof, or reagents thereof, as described herein, only require two temperatures to be operable. In some instances, devices, systems, kits, components thereof, or reagents thereof, as described herein, only require three temperatures to be operable.

In some instances, temperature at which the devices, systems, kits, components thereof, or reagents thereof are operable at a temperature range or at least one temperature that falls within a temperature range. In some instances, the range of temperatures is about −50° C. to about 100° C. In some instances, the range of temperatures is about −50° C. to about 90° C. In some instances, the range of temperatures is about −50° C. to about 80° C. In some instances, the range of temperatures is about is about −50° C. to about 70° C. In some instances, the range of temperatures is about −50° C. to about 60° C. In some instances, the range of temperatures is about −50° C. to about 50° C. In some instances, the range of temperatures is about −50° C. to about 40° C. In some instances, the range of temperatures is about −50° C. to about 30° C. In some instances, the range of temperatures is about −50° C. to about 20° C. In some instances, the range of temperatures is about −50° C. to about 10° C. In some instances, the range of temperatures is about 0° C. to about 100° C. In some instances, the range of temperatures is about 0° C. to about 90° C. In some instances, the range of temperatures is about 0° C. to about 80° C. In some instances, the range of temperatures is about is about 0° C. to about 70° C. In some instances, the range of temperatures is about 0° C. to about 60° C. In some instances, the range of temperatures is about 0° C. to about 50° C. In some instances, the range of temperatures is about 0° C. to about 40° C. In some instances, the range of temperatures is about 0° C. to about 30° C. In some instances, the range of temperatures is about 0° C. to about 20° C. In some instances, the range of temperatures is about 0° C. to about 10° C. In some instances, the range of temperatures is about 15° C. to about 100° C. In some instances, the range of temperatures is about 15° C. to about 90° C. In some instances, the range of temperatures is about 15° C. to about 80° C. In some instances, the range of temperatures is about is about 15° C. to about 70° C. In some instances, the range of temperatures is about 15° C. to about 60° C. In some instances, the range of temperatures is about 15° C. to about 50° C. In some instances, the range of temperatures is about 15° C. to about 40° C. In some instances, the range of temperatures is about 15° C. to about 30° C. In some instances, the range of temperatures is about 10° C. to about 30° C. In some instances, devices, systems, kits disclosed herein, including all components thereof, and all reagents thereof, are completely operable at room temperature, not requiring cooling, freezing or heating.

In some instances, devices, systems, and kits disclosed herein comprise a heating device or a cooling device to allow a user to obtain the at least one temperature or temperature range. Non-limiting examples of heating devices and cooling devices are pouches or bags of material that can be cooled in a refrigerator or freezer, or heated with a microwave, oven or stove top. In some instances, the heating or cooling device is plugged into an electrical socket, and subsequently applied to devices disclosed herein or components thereof, thereby transmitting heat to the device or component thereof or cooling the device or component thereof. Another non-limiting example of a heating device is an electrical wire or coil that runs through the device or portion thereof. The electrical wire or coil may be activated by external (e.g. solar, outlet) or internal (e.g., battery) power to convey heat to the device or portion thereof. In some instances, devices, systems, kits disclosed herein comprise a thermometer or temperature indicator to assist a user with determining that a suitable temperature or temperature range has been obtained for the device, system or component thereof. Alternatively, or additionally, the user employs a device in a typical home setting (e.g., thermometer, cell phone, etc.) to assess the temperature.

In some instances, devices, systems and kits disclosed herein detect components of the biological sample or products thereof (e.g., amplification products, conjugation products, binding products) within a time range of receiving the biological sample. In some instances, detecting occurs via a signaling molecule described herein. In some instances, the time range is about one second to about one minute. In some instances, the time range is about ten seconds to about one minute. In some instances, the time range is about twenty seconds to about one minute. In some instances, the time range is about thirty seconds to about one minute. In some instances, the time range is about 10 seconds to about 2 minutes. In some instances, the time range is about 10 seconds to about 3 minutes. In some instances, the time range is about 10 seconds to about 5 minutes. In some instances, the time range is about 10 seconds to about 10 minutes. In some instances, the time range is about 10 seconds to about 15 minutes. In some instances, the time range is about 10 seconds to about 20 minutes. In some instances, the time range is about 30 seconds to about 2 minutes. In some instances, the time range is about 30 seconds to about 5 minutes. In some instances, the time range is about 30 seconds to about 10 minutes. In some instances, the time range is about 30 seconds to about 15 minutes. In some instances, the time range is about 30 seconds to about 20 minutes. In some instances, the time range is about 30 seconds to about 30 minutes. In some instances, the time range is about 1 minute to about 2 minutes. In some instances, the time range is about 1 minute to about 5 minutes. In some instances, the time range is about 1 minute to about 10 minutes. In some instances, the time range is about 1 minute to about 20 minutes. In some instances, the time range is about 1 minute to about 30 minutes. In some instances, the time range is about 5 minute to about 10 minutes. In some instances, the time range is about 5 minute to about 15 minutes. In some instances, the time range is about 5 minute to about 20 minutes. In some instances, the time range is about 5 minute to about 30 minutes. In some instances, the time range is about 5 minute to about 60 minutes.

In some instances, devices, systems and kits disclosed herein detect a component of a biological sample or a product thereof (e.g., amplification product, conjugation product, binding product) in less than a given amount of time. In some instances, devices, systems and kits disclosed herein provide an analysis of a component of a biological sample or product thereof in less than a given amount of time. In some instances, the amount of time is less than 1 minute. In some instances, the amount of time is less than 5 minutes. In some instances, the amount of time is less than 10 minutes. In some instances, the amount of time is less than 15 minutes. In some instances, the amount of time is less than 20 minutes. In some instances, the amount of time is less than 30 minutes. In some instances, the amount of time is less than 60 minutes. In some instances, the amount of time is less than 2 hours. In some instances, the amount of time is less than 8 hours.

### Communication & Information Storage

Preferably, devices, systems and kits disclosed herein comprise a communication connection or interface so that genetic information obtained can be shared with others not physically present. The communication connection or interface may also allow for input from other sources. In some instances, devices, systems and kits disclosed herein comprise an interface for receiving information based on the genetic information obtained. The interface or communication connection may also receive non-genetic information from the user (e.g., medical history, medical conditions, age, weight, etc.). The interface or communication connection may also receive information provided by someone or something other than the user. By way of non-limiting example, this includes web-based information, information from a medical practitioner, and information from an insurance company. For example, devices, systems and kits disclosed herein may comprise, or communicate with, an artificial intelligence interface that markets gender-related or gender-specific products to a pregnant subject based on a gender result of the test. In some instances, devices, systems and kits disclosed herein comprise an information storage unit, e.g., a computer chip. In some instances, the devices, systems and kits disclosed herein comprise means to store genetic information securely. For example, devices, systems and kits disclosed herein may comprise a data chip or a connection (wired or wireless) to a hard drive, server, database or cloud.

In some instances, the devices, systems and kits disclosed herein are capable of communicating information about biomarkers in the biological sample to a communication device. In some instances the communication device is connected to the internet. In some instances the communication device is not connected to the internet. In some instances, devices, systems and kits disclosed herein are capable of communicating information about biomarkers in the biological sample through the communication device to the internet. Non-limiting examples of communication devices are cell phones, electronic notepads, and computers.

In some instances, devices, systems and kits disclosed herein are capable of identifying and storing intermediate results of a corresponding test. Intermediate results may be indicative of which test parameters (e.g., analytes, reagents, labels, methods, or device components) were useful or accurate. This information may be useful feedback to a team developing a test or assay with devices, systems and kits disclosed herein. A team receiving this feedback may choose new, better or optimal parameters based on this information or be reassured that they have chosen optimal parameters.

In some instances, devices, systems and kits disclosed herein comprise a communication connection or a communication interface. In some embodiments, the communication interface provides a wired interface. In further embodiments, the wired communications interface utilizes Universal Serial Bus (USB) (including mini-USB, micro-USB, USB Type A, USB Type B, and USB Type C), IEEE 1394 (FireWire), Thunderbolt, Ethernet, and optical interconnect.

In some embodiments, the communication interface provides a wireless interface. In further embodiments, the wireless communications interface utilizes a wireless communications protocol such as infrared, near-field communications (NFC) (including RFID), Bluetooth, Bluetooth Low Energy (BLE), ZigBee, ANT, IEEE 802.11 (Wi-Fi), Wireless Local Area Network (WLAN), Wireless Personal Area Network (WPAN), Wireless Wide Area Network (WWAN), WiMAX, IEEE 802.16 (Worldwide Interoperability for Microwave Access (WiMAX)), or 3G/4G/LTE/5G cellular communication methods.

In some embodiments, devices, systems, kits, and methods described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device includes a communication interface (e.g., network adapter) for communicating with one or more peripheral devices, one or more distinct digital processing devices, one or more computing systems, one or more computer networks, and/or one or more communications networks.

In some embodiments, the digital processing device is communicatively coupled to a computer network (“network”) with the aid of the communication interface. Suitable networks include, a personal area network (PAN), a local area networks (LAN), a wide area network (WAN), an intranet, an extranet, the Internet (providing access to the World Wide Web) and combinations thereof. The network in some cases is a telecommunication and/or data network. The network, in various cases, includes one or more computer servers, which enable distributed computing, such as cloud computing. The network, in some cases and with the aid of the device, implements a peer-to-peer network, which enables devices coupled to the device to behave as a client or a server.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, and personal digital assistants. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. In some instances, the operating system comprises an Internet of Things (IoT) device. Non-limiting examples of an IoT device include Amazon's Alexa®, Microsoft's Cortana®, Apple Home Pod®, and Google Speaker®. In some instances, devices, systems, and kits disclosed herein comprise a virtual reality and/or augmented reality system.

In some embodiments, devices, systems, and kits disclosed herein comprise a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with the digital processing device, such as a VR headset.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

### Mobile Application

In some embodiments, devices, systems, kits, and methods disclosed herein comprise a digital processing device, or use of the same, wherein the digital processing device is provided with executable instructions in the form of a mobile application. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, and Samsung® Apps.

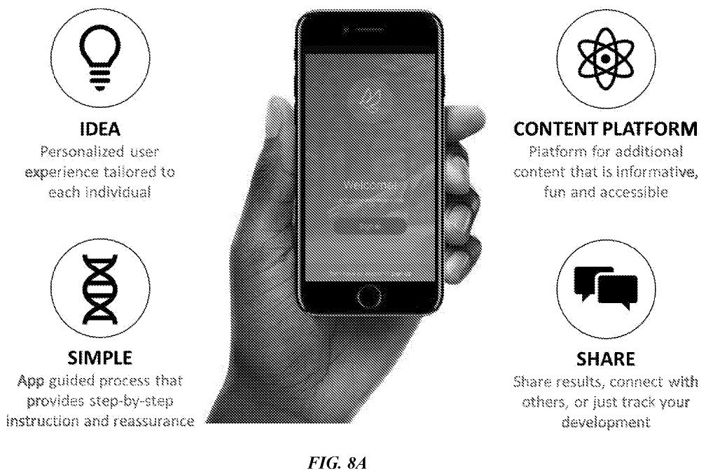

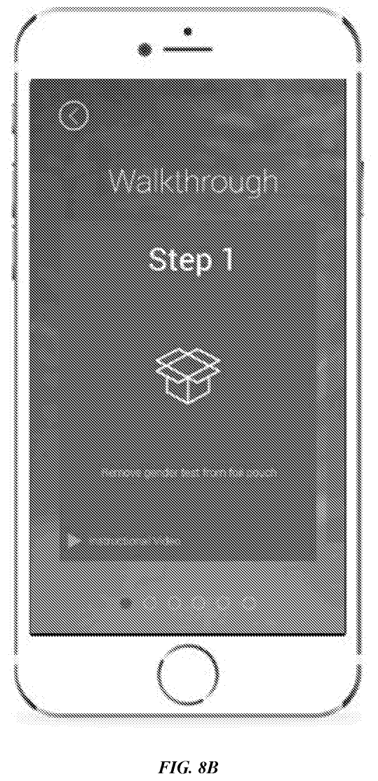

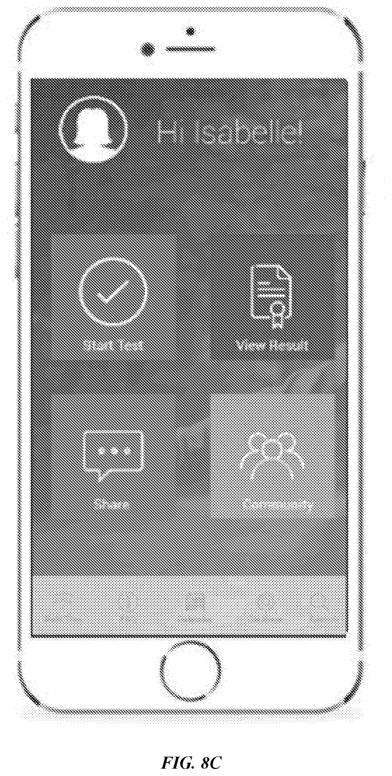

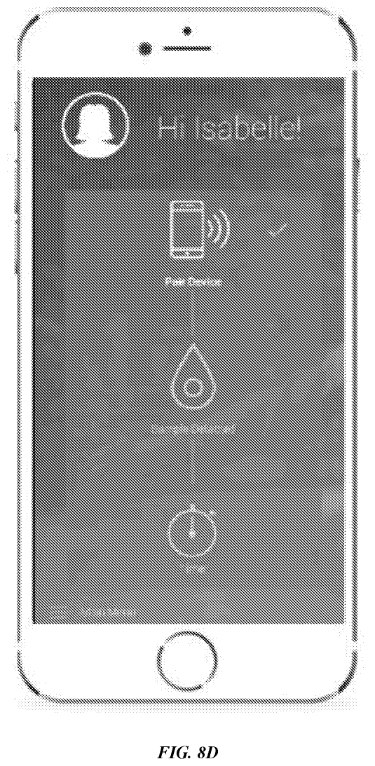

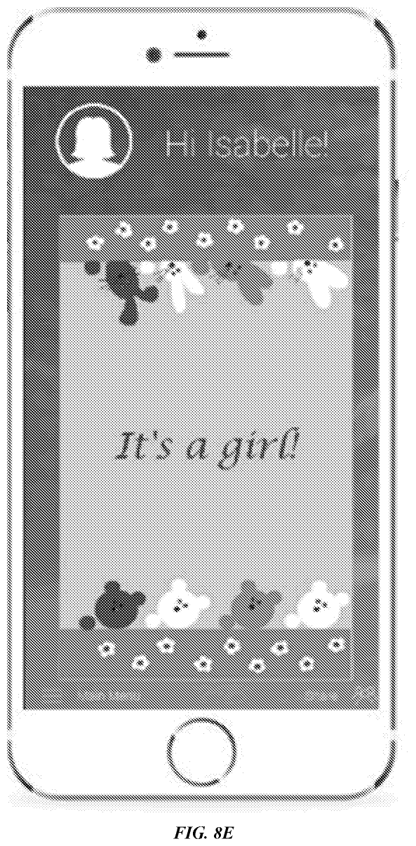

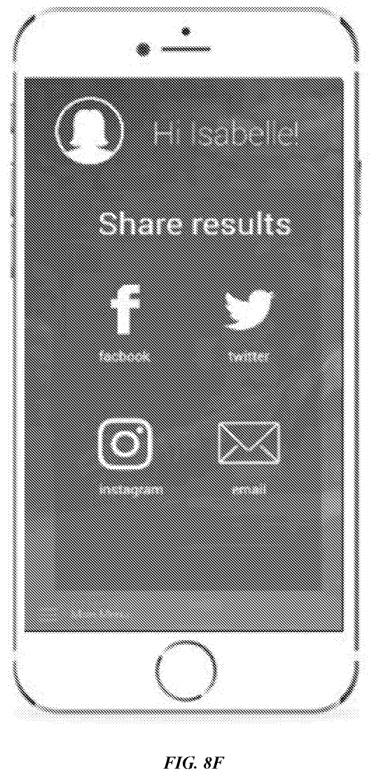

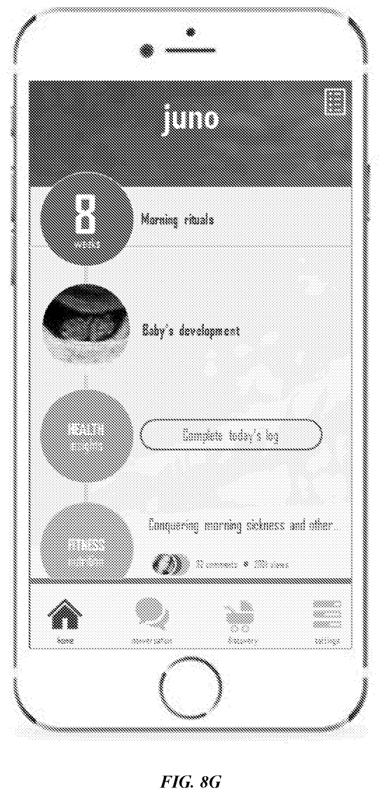

Referring to FIG. 8A, in a particular embodiment, a mobile application is configured to connect with, communicate with, and receive genetic information and other information from the devices, systems and kits disclosed herein. FIG. 8A is a diagram depicting various functions that the mobile application optionally provides to users. In this embodiment, the mobile application optionally provides: 1) a personalized, tailored user experience (UX) based on the personal information and preferences of the user; 2) an interactive text-, audio-, and/or video-driven instructional experience to inform the user how to utilize the devices, systems, and kits; 3) a content platform that provides the user with access to articles, news, media, games, and the like; and 4) tools for tracking and sharing information, test results, and events.

Referring to FIG. 8B, in a particular embodiment, the mobile application optionally includes an interactive interface providing a step-by-step walkthrough to guide a user through use of the devices, systems and kits disclosed herein. In various embodiments, the interactive walkthrough includes text, images, animations, audio, video, and the like to inform and instruct the user.

Referring to FIG. 8C, in a particular embodiment, the mobile application optionally includes a home screen allowing a user to access the mobile application functionality disclosed herein. In this embodiment, the home screen includes a personalized greeting as well as interface elements allowing the user to start a test, view current and historic test results, share test results, and interact with a larger community of users.

Referring to FIG. 8D, in a particular embodiment, the mobile application optionally includes a progress diagram informing a user of the status of a process for connecting to a device, system, or kit disclosed herein. In this embodiment, the diagram shows all the steps and indicates the current step. The steps are: 1) pair with the device via, for example, Bluetooth; 2) detect a sample in the device; and 3) wait for the sample to be processed. In some embodiments, the diagram is interactive, animated, or augmented with media or other content.

Referring to FIG. 8E, in a particular embodiment, the mobile application optionally includes a test report, which is provided to the user to communicate the results of a test. In this example, the user is provided with a report letting her know that she is pregnant with a daughter. In some embodiments, the report is interactive, animated, or augmented with media or other content, which may be personalized based on the results of the test.

Referring to FIG. 8F, in a particular embodiment, the mobile application optionally includes a social sharing screen allowing a user to access features to share test results. Many services, platforms, and networks are suitable for sharing test results and other information and events. Suitable social networking and sharing platforms include, by way of non-limiting examples, Facebook, YouTube, Twitter, LinkedIn, Pinterest, Google Plus+, Tumblr, Instagram, Reddit, VK, Snapchat, Flickr, Vine, Meetup, Ask.fm, Classmates, QQ, WeChat, Swarm by Foursquare, Kik, Yik Yak, Shots, Periscope, Medium, Soundcloud, Tinder, WhatsApp, Snap Chat, Slack, Musical.ly, Peach, Blab, Renren, Sina Weibo, Renren, Line, and Momo. In some embodiments, the test results are shared by SMS, MMS or instant message. In some embodiments, the test results are shared by email.

Referring to FIG. 8G, in a particular embodiment, the mobile application optionally includes a home screen allowing a user to access additional features such as a blog and timeline of important information and events related to the test results, which is optionally shared. In various embodiments, suitable information and events include those pertaining to clinical trial outcomes, newly marketed therapeutics, nutrition, exercise, fetal development, health, etc. In the case of a pregnant subject, the information and events are organized into a timeline interface based on time point (e.g., number of weeks) in the pregnancy. In this embodiment, the home screen further includes access to user preferences and settings.

In some instances, devices, systems and kits disclosed herein are used according to the following methods.

### II. Methods

In some aspects, the following disclosed methods employ the foregoing described devices, systems and kits. In general, methods disclosed herein comprise obtaining a biological sample and detecting a component thereof. Obtaining the biological sample may occur in a clinical or laboratory setting. Alternatively, obtaining may occur at a location remote from a clinical or laboratory setting, such as, by way of non-limiting example, a home, a school, a farm, or a battlefield. In some instances, detecting occurs in a clinical or laboratory setting. In other instances, detecting occurs at a location remote from a clinical or laboratory setting. Other steps of the methods disclosed herein, e.g., amplifying a nucleic acid, may occur in the clinical/laboratory setting or at a remote location.

In general, methods disclosed herein comprise collecting and analyzing a relatively small volume of a biological sample. By way of non-limiting example, disclosed herein are methods comprising obtaining a sample from a female subject in a non-laboratory setting, wherein the volume of the biological sample is not greater than about 300 μl; amplifying at least one circulating cell free nucleic acid in the sample to produce at least one amplification product; detecting the presence or absence of an amplification product comprising a sequence corresponding to a Y chromosome.

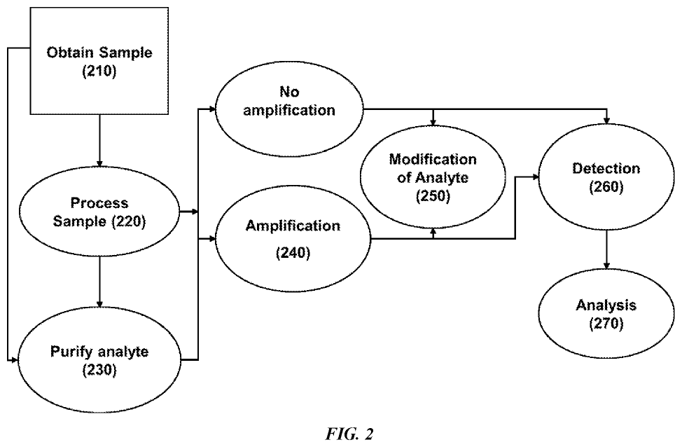

FIG. 2 shows a general flow chart with various routes that methods, devices and systems disclosed herein can follow. Initially a sample is obtained in step 210. A minimal amount of sample must be obtained in order to gather useful information from the sample. The sample may be a biological sample disclosed herein. The sample may be a crude, unprocessed sample (e.g., whole blood). The sample may be a processed sample (e.g., plasma). The amount of sample is likely based on the sample type. Typically, the sample is processed or an analyte (e.g., a nucleic acid or other biomarker) is purified from the sample in step 220 to produce an analyte that can be amplified and/or detected. Processing may comprise filtering a sample, binding a component of the sample that contains an analyte, binding the analyte, stabilizing the analyte, purifying the analyte, or a combination thereof. Non-limiting examples of sample components are cells, viral particles, bacterial particles, exosome, and nucleosomes. In some instances, the analyte is a nucleic acid and it is amplified to produce an amplicon for analysis in step 240. In other instances, the analyte may or may not be a nucleic acid, but regardless is not amplified. The analyte or amplicon is optionally modified (250) before detection and analysis in steps 260 and 270, respectively. In some instances, modification occurs during amplification (not shown). For example, the analyte or amplicon may be tagged or labeled. Detection may involve sequencing, target-specific probes, isothermal amplification and detection methods, quantitative PCR, or single molecule detection. FIG. 2 is provided as a broad overview of devices and methods disclosed herein, but devices and methods disclosed herein are not limited by FIG. 2. Devices and methods may comprise additional components and steps, respectively that are not shown in FIG. 2.

Sample Collection

In some instances, methods disclosed herein comprise obtaining a biological sample described herein. Non-limiting examples of biological samples include blood, plasma, urine, saliva, vaginal fluid, interstitial fluid. In some instances, methods disclosed herein comprise obtaining a environmental sample described herein. Non-limiting examples of environmental samples include waste water, ocean water, food and beverages.

In some instances, methods disclosed herein are performed in a single location, e.g., from obtaining to detecting. In some instances, the single location is a home. In some instances, the single location is not a medical, technical or pathology laboratory. In some instances, methods disclosed herein are performed entirely by the female subject. In some instances, methods disclosed herein are performed by a subject that does not receive any technical training to perform the method. In some instances, methods disclosed herein are performed by a subject that does not receive any technical training to perform the method, other than an instruction set provided with a device used to perform the methods.

In some instances, methods disclosed herein are performed with a device, system or kit described herein. In some instances, methods disclosed herein are performed away from a clinical setting, such as, by way of non-limiting example, a medical clinic, a hospital, a scientific research laboratory, a pathology laboratory, or a clinical test laboratory. In some instances, methods disclosed herein are performed in a home, in a school, or in a family planning center. In some instances, the methods may be performed by the subject. In some instances, methods disclosed herein are performed by a user that has not received any technical training necessary to perform the method.

In general, methods disclosed herein comprise obtaining, processing, and/or analyzing a relatively small volume of a biological sample. A small volume may also be referred to as a small input, low input, or a low volume. In some instances, methods disclosed herein comprise obtaining a volume of the biological sample, wherein the volume is less than one milliliter. In some instances, methods disclosed herein are performed with not more than one milliliter of the biological sample. In some instances, methods disclosed herein are performed with not more than 100 μl of the biological sample. In some instances, methods disclosed herein comprise obtaining a volume of the biological sample, wherein the volume falls within a range of sample volumes. In some instances, the range of sample volumes is about 1 μl to about one milliliter. In some instances, the range of sample volumes is about 5 μl to about one milliliter. In some instances, the range of sample volumes is about 1 μl to about 900 μl. In some instances, the range of sample volumes is about 1 μl to about 800 μl. In some instances, the range of sample volumes is about 1 μl to about 700 μl. In some instances, the range of sample volumes is about 1 μl to about 600 μl. In some instances, the range of sample volumes is about 1 μl to about 500 μl. In some instances, the range of sample volumes is about 1 μl to about 400 μl. In some instances, the range of sample volumes is about 1 μl to about 300 μl. In some instances, the range of sample volumes is about 1 μl to about 200 μl. In some instances, the range of sample volumes is about 1 μl to about 150 μl. In some instances, the range of sample volumes is 1 μl to about 100 μl. In some instances, the range of sample volumes is about 1 μl to about 90 μl. In some instances, the range of sample volumes is about 1 μl to about 85 μl. In some instances, the range of sample volumes is about 1 μl to about 80 μl. In some instances, the range of sample volumes is about 1 μl to about 75 μl. In some instances, the range of sample volumes is about 1 μl to about 70 μl. In some instances, the range of sample volumes is about 1 μl to about 65 μl. In some instances, the range of sample volumes is about 1 μl to about 60 μl. In some instances, the range of sample volumes is about 1 μl to about 55 μl. In some instances, the range of sample volumes is about 1 μl to about 50 μl. In some instances, the range of sample volumes is about 5 μl to about 45 μl. In some instances, the range of sample volumes is about 5 μl to about 40 μl. In some instances, the range of sample volumes is about 15 μl to about 150 μl. In some instances, the range of sample volumes is 15 μl to about 100 μl. In some instances, the range of sample volumes is about 15 μl to about 90 μl. In some instances, the range of sample volumes is about 15 μl to about 85 μl. In some instances, the range of sample volumes is about 15 μl to about 80 μl. In some instances, the range of sample volumes is about 15 μl to about 75 μl. In some instances, the range of sample volumes is about 15 μl to about 70 μl. In some instances, the range of sample volumes is about 15 μl to about 65 μl. In some instances, the range of sample volumes is about 15 μl to about 60 μl. In some instances, the range of sample volumes is about 15 μl to about 55 μl. In some instances, the range of sample volumes is about 15 μl to about 50 μl. In some instances, the range of sample volumes is about 10 μl to about 45 μl. In some instances, the range of sample volumes is about 10 μl to about 40 μl.

In some aspects, described herein are methods comprising: obtaining a fluid sample from a subject with a handheld device, wherein the volume of the fluid sample is not greater than about 300 μL; sequencing at least one cell free nucleic acid in the fluid sample with the handheld device; detecting the presence or absence of a sequence corresponding to a sequence of interest through a display in the handheld device, thereby determining genetic information about the subject; and communicating, with the handheld device, the genetic information to another subject. In some instances, the detecting and communicating occur simultaneously. In some instances, the volume is not greater than 250 μL. In some instances, the volume is not greater than 200 μL. In some instances, the volume is not greater than 150 μL. In some instances, the volume is not greater than 140 μL. In some instances, the volume is not greater than 130 μL. In some instances, the volume is not greater than 120 μL. In some instances, the volume is not greater than 100 μL.

In some instances, methods disclosed herein comprise obtaining a blood sample. In some instances, obtaining blood does not comprise a phlebotomy. In some instances, the subject performs the obtaining by pressing his/her skin against a transdermal puncture device of the handheld device. In general, a transdermal puncture device comprises at least one needle, microneedle, or needle array. In some instances, the subject presses a finger, toe, arm, shoulder, or palm against the transdermal device. In some instances, the subject presses a finger against the transdermal puncture device. In some instances, the subject presses his/her skin against the transdermal puncture device not more than once. In some instances, the subject presses his/her skin against the transdermal puncture device not more than twice. In some instances, methods comprise obtaining a blood sample and sending the blood sample or a component thereof (e.g., plasma/serum) to a location remote from the site of the obtaining step (e.g., laboratory, clinic or research center) for additional processing and analysis. In other instances, the methods comprise detecting a test result at the site of the obtaining step using, e.g., a device disclosed herein.

In some instances, methods disclosed herein comprise obtaining a blood sample via a finger prick. In some instances, methods disclosed herein comprise obtaining a blood sample via multiple finger pricks. In some instances, methods disclosed herein comprise obtaining a blood sample from not more than 2 finger pricks. In some instances, methods disclosed herein comprise obtaining a blood sample from not more than 3 finger pricks. In some instances, methods disclosed herein comprise obtaining a blood sample via a single finger prick. In some instances, methods disclosed herein comprise obtaining a blood sample with not more than a single finger prick. In some instances, methods disclosed herein comprise obtaining capillary blood (e.g., blood obtained from a finger). In some instances, methods comprise squeezing or milking blood from a prick to obtain a desired volume of blood. While a finger prick is a common method for obtaining capillary blood, other locations on the body would also be suitable, e.g., a toe, palm, heel, arm, shoulder. In some instances, methods disclosed herein comprise obtaining a blood sample without a phlebotomy. In some instances, methods disclosed herein do not comprise obtaining venous blood (e.g., blood obtained from a vein).

In some instances, methods disclosed herein comprise obtaining at least about 1 μL of blood to provide a test result with at least about 90% confidence or accuracy. In some instances, the devices, systems and kits disclosed herein require at least about 5 μL of blood to provide a test result with at least about 90% confidence or accuracy. In some instances, the devices, systems and kits disclosed herein require at least about 15 μL of blood to provide a test result with at least about 90% confidence or accuracy. In some instances, the devices, systems and kits disclosed herein require at least about 15 μL of blood to provide a test result with at least about 90% confidence or accuracy. In some instances, the devices, systems and kits disclosed herein require at least about 20 μL of blood to provide a test result with at least about 90% confidence or accuracy. In some instances, the devices, systems and kits disclosed herein require at least about 20 μL of blood to provide a test result with at least about 95% confidence or accuracy. In some instances, the devices, systems and kits disclosed herein require at least about 20 μL of blood to provide a test result with at least about 99% confidence or accuracy. In some instances, the devices, systems and kits disclosed herein require only about 20 μL to about 100 μL of blood to provide a test result with at least about 90% confidence or accuracy. In some instances, the devices, systems and kits disclosed herein require only about 20 μL to about 100 μL of blood to provide a test result with at least about 95% confidence or accuracy. In some instances, the devices, systems and kits disclosed herein require only about 20 μL to about 100 μL of blood to provide a test result with at least about 99% confidence or accuracy.

In some instances, methods disclosed herein comprise separating a cell from a biological sample. In some instances, methods disclosed herein comprise separating a fraction of a sample that does not contain cells from a fraction of a sample that does contain cells. Methods may comprise processing the cells and/or analyzing contents of the cells. Processing or analyzing may occur within a device or system disclosed herein. In some instances, cells are preserved or saved for subsequent analysis outside of the device or system.

In some instances, methods disclosed herein comprise obtaining plasma. Plasma makes up roughly 55% of whole blood. In some instances, the devices, systems, kits, and methods disclosed herein require at least about 3 μL of plasma to provide a test result with at least about 90% confidence or accuracy. In some instances, the devices, systems and kits disclosed herein require at least about 8 μL of plasma to provide a test result with at least about 90% confidence or accuracy. In some instances, the devices, systems and kits disclosed herein require at least about 8 μL of plasma to provide a test result with at least about 90% confidence or accuracy. In some instances, the devices, systems and kits disclosed herein require at least about 12 μL of plasma to provide a test result with at least about 90% confidence or accuracy. In some instances, the devices, systems and kits disclosed herein require at least about 12 μL of plasma to provide a test result with at least about 95% confidence or accuracy. In some instances, the devices, systems and kits disclosed herein require at least about 12 μL of plasma to provide a test result with at least about 99% confidence or accuracy. In some instances, the devices, systems and kits disclosed herein require only about 12 μL to about 60 μL of plasma to provide a test result with at least about 90% confidence or accuracy. In some instances, the devices, systems and kits disclosed herein require only about 12 μL to about 60 μL of plasma to provide a test result with at least about 95% confidence or accuracy. In some instances, the devices, systems and kits disclosed herein require only about 12 μL to about 60 μL of plasma to provide a test result with at least about 99% confidence or accuracy.

In some instances, the biological sample evaluated using the methods, devices, systems and kits disclosed herein is urine, and the volume of urine used is about 0.25 μl to 1 milliliter. In some instances, the volume of urine used is about 0.25 μl to about 1 milliliter. In some instances, the volume of urine used is at least about 0.25 μl. In some instances, the volume of urine used is at most about 1 milliliter. In some instances, the volume of urine used is about 0.25 μl to about 0.5 μl, about 0.25 μl to about 0.75 μl, about 0.25 μl to about 1 μl, about 0.25 μl to about 5 μl, about 0.25 μl to about 10 μl about 0.25 μl to about 50 μl about 0.25 μl to about 100 μl about 0.25 μl to about 150 μl about 0.25 μl to about 200 μl about 0.25 μl to about 500 μl about 0.25 μl to about 1 milliliter, about 0.5 μl to about 0.75 μl about 0.5 μl to about 1 μl about 0.5 μl to about 5 μl about 0.5 μl to about 10 μl about 0.5 μl to about 50 μl about 0.5 μl to about 100 about 0.5 μl to about 150 μl about 0.5 μl to about 200 μl about 0.5 μl to about 500 μl about 0.5 μl to about 1 milliliter, about 0.75 μl to about 1 μl about 0.75 μl to about 5 μl about 0.75 μl to about 10 μl about 0.75 μl to about 50 μl about 0.75 μl to about 100 μl about 0.75 μl to about 150 μl, about 0.75 μl to about 200 μl about 0.75 μl to about 500 μl about 0.75 μl to about 1 milliliter, about 1 μl to about 5 μl, about 1 μl to about 10 μl, about 1 μl to about 50 μl, about 1 μl to about 100 μl, about 1 μl to about 150 μl, about 1 μl to about 200 μl, about 1 μl to about 500 μl, about 1 μl to about 1 milliliter, about 5 μl to about 10 μl, about 5 μl to about 50 μl, about 5 μl to about 100 μl, about 5 μl to about 150 μl, about 5 μl to about 200 μl, about 5 μl to about 500 μl, about 5 μl to about 1 milliliter, about 10 μl to about 50 μl, about 10 μl to about 100 μl, about 10 μl to about 150 μl, about 10 μl to about 200 μl, about 10 μl to about 500 μl, about 10 μl to about 1 milliliter, about 50 μl to about 100 μl, about 50 μl to about 150 μl, about 50 μl to about 200 μl, about 50 μl to about 500 μl, about 50 μl to about 1 milliliter, about 100 μl to about 150 μl, about 100 μl to about 200 μl, about 100 μl to about 500 μl, about 100 μl to about 1 milliliter, about 150 μl to about 200 μl, about 150 μl to about 500 μl, about 150 μl to about 1 milliliter, about 200 μl to about 500 μl, about 200 μl to about 1 milliliter, or about 500 μl to about 1 milliliter. In some instances, the volume of urine used is about 0.25 μl, about 0.5 μl, about 0.75 μl, about 1 μl, about 5 μl, about 10 μl, about 50 μl about 100 μl about 150 μl about 200 μl, about 500 μl, or about 1 milliliter.

In some instances, methods disclosed herein comprise obtaining a biological sample from the female subject, wherein the biological sample contains an amount of cell free nucleic acids. In some instances, the cell free nucleic acids comprise DNA. In some instances, the cell free nucleic acids comprise RNA. In some instances, the cell free nucleic acids comprise DNA and RNA. In some instances, the cell free nucleic acids comprise cell free fetal nucleic acids. In some instances, the amount of cell free nucleic acids falls within a range. In some instances, the range is about 1 pg to about 10 pg. In some instances, the range is about 1 pg to about 50 pg. In some instances, the range is about 1 pg to about 100 pg. In some instances, the range is about 1 pg to about 1 ng. In some instances, the range is about 2 pg to about 10 pg. In some instances, the range is about 1 pg to about 1 ng. In some instances, the range is about 2 pg to about 100 pg. In some instances, the range is about 3 pg to about 10 pg. In some instances, the range is about 3 pg to about 30 pg. In some instances, the range is about 3 pg to about 100 pg. In some instances, the range is about 3 pg to about 300 pg. In some instances the range is about 3 pg to about 1 ng. In some instances the range is about 3 pg to about 2 ng. In some instances the range is about 3 pg to about 3 ng. In some instances the range is about 3 pg to about 4 ng. In some instances the range is about 3 pg to about 5 ng. In some instances the range is about 3 pg to about 10 ng. In some instances, methods comprise obtaining less than about 10 ng of cell free fetal nucleic acids. In some instances, methods comprise obtaining less than about 7 ng of cell free fetal nucleic acids. In some instances, methods comprise obtaining less than about 5 ng of cell free fetal nucleic acids. In some instances, methods comprise obtaining less than about 1 ng of cell free fetal nucleic acids. In some instances, methods comprise obtaining not more than about 10 ng of cell free fetal nucleic acids. In some instances, methods comprise obtaining not more than about 7 ng of cell free fetal nucleic acids. In some instances, methods comprise obtaining not more than about 5 ng of cell free fetal nucleic acids. In some instances, methods comprise obtaining not more than about 1 ng of cell free fetal nucleic acids.

In some instances, methods disclosed herein comprise obtaining a biological sample from the female subject, wherein the biological sample contains at least one cell free fetal nucleic acid comprising a sequence unique to a Y chromosome. In some instances, methods disclosed herein comprise obtaining a biological sample from the female subject, wherein the biological sample contains about 1 to about 5 cell free fetal nucleic acids comprising a sequence unique to a Y chromosome. In some instances, methods disclosed herein comprise obtaining a biological sample from the female subject, wherein the biological sample contains about 1 to about 15 cell free fetal nucleic acids comprising a sequence unique to a Y chromosome. In some instances, methods disclosed herein comprise obtaining a biological sample from the female subject, wherein the biological sample contains about 1 to about 25 cell free fetal nucleic acids comprising a sequence unique to a Y chromosome. In some instances, methods disclosed herein comprise obtaining a biological sample from the female subject, wherein the biological sample contains about 1 to about 100 cell free fetal nucleic acids comprising a sequence unique to a Y chromosome. In some instances, methods disclosed herein comprise obtaining a biological sample from the female subject, wherein the biological sample contains about 5 to about 100 cell free fetal nucleic acids comprising a sequence unique to a Y chromosome.

By way of non-limiting example, methods may comprise obtaining a fluid sample from a female pregnant subject with a handheld device, wherein the volume of the fluid sample is not greater than about 300 μL; sequencing at least one cell free nucleic acid in the fluid sample with the handheld device; detecting the presence or absence of a sequence corresponding to a Y chromosome through a display in the handheld device, thereby determining a gender of a fetus in the female pregnant subject; and communicating, with the handheld device, the gender to another subject. In some instances, the volume of the biological sample is not greater than about 120 μl. In some instances, the methods comprise detecting sequencing reads corresponding to the Y chromosome.

Also by way of non-limiting example, methods may comprise obtaining a biological sample from a female subject, wherein the volume of the biological sample is not greater than about 120 μl; contacting the sample with an oligonucleotide primer comprising a sequence corresponding to a Y chromosome for amplifying at least one circulating cell free nucleic acid in the sample; detecting an absence of an amplification product, thereby indicating that the fetus is female. Obtaining, contacting and detecting may occur with a single device.

### Isolating and Purifying Nucleic Acids & Other Biomarkers

In some instances, methods disclosed herein comprise isolating or purifying nucleic acids from one or more non-nucleic acid components of a biological sample. Non-nucleic acid components may also be considered unwanted substances. Non-limiting examples of non-nucleic acid components include cells (e.g., blood cells), cell fragments, extracellular vesicles, lipids, proteins or a combination thereof. Additional non-nucleic acid components are described herein and throughout. It should be noted that while methods may comprise isolating/purifying nucleic acids, they may also comprise analyzing a non-nucleic acid component of a sample that is considered an unwanted substance in a nucleic acid purifying step. Isolating or purifying may comprise removing components of a biological sample that would inhibit, interfere with or otherwise be detrimental to the later process steps such as nucleic acid amplification or detection.

Isolating or purifying may be performed with a device or system disclosed herein. Isolating or purifying may be performed within a device or system disclosed herein. Isolating and/or purifying may occur with the use of a sample purifier disclosed herein. In some instances, isolating or purifying nucleic acids comprises removing non-nucleic acid components from a biological sample described herein. In some instances, isolating or purifying nucleic acids comprises discarding non-nucleic acid components from a biological sample. In some instances, isolating or purifying comprises collecting, processing and analyzing the non-nucleic acid components. In some instances, the non-nucleic acid components may be considered biomarkers because they provide additional information about the subject.

In some instances, isolating or purifying nucleic acids comprise lysing a cell. In some instances, isolating or purifying nucleic acids avoids lysing a cell. In some instances, isolating or purifying nucleic acids does not comprise lysing a cell. In some instances, isolating or purifying nucleic acids does not comprise an active step intended to lyse a cell. In some instances, isolating or purifying nucleic acids does not comprise intentionally lysing a cell. Intentionally lysing a cell may include mechanically disrupting a cell membrane (e.g., shearing). Intentionally lysing a cell may include contacting the cell with a lysis reagent. Exemplary lysis reagents are described herein.

In some instances, isolating or purifying nucleic acids comprises lysing and performing sequence specific capture of a target nucleic acid with “bait” in a solution followed by binding of the “bait” to solid supports such as magnetic beads, e.g. Legler et al., Specific magnetic bead-based capture of free fetal DNA from maternal plasma, Transfusion and Apheresis Science 40 (2009), 153-157. In some instances, methods comprise performing sequence specific capture in the presence of a recombinase or helicase. Use of a recombinase or helicase may avoid the need for heat denaturation of a nucleic acid and speed up the detection step.

In some instances, isolating or purifying comprises separating components of a biological sample disclosed herein. By way of non-limiting example, isolating or purifying may comprise separating plasma from blood. In some instances, isolating or purifying comprises centrifuging the biological sample. In some instances, isolating or purifying comprises filtering the biological sample in order to separate components of a biological sample. In some instances, isolating or purifying comprises filtering the biological sample in order to remove non-nucleic acid components from the biological sample. In some instances, isolating or purifying comprises filtering the biological sample in order to capture nucleic acids from the biological sample.

In some instances, the biological sample is blood and isolating or purifying a nucleic acid comprises obtaining or isolating plasma from blood. Obtaining plasma may comprise separating plasma from cellular components of a blood sample. Obtaining plasma may comprise centrifuging the blood, filtering the blood, or a combination thereof. Obtaining plasma may comprise allowing blood to be subjected to gravity (e.g., sedimentation). Obtaining plasma may comprise subjecting blood to a material that wicks a portion of the blood away from non-nucleic acid components of the blood. In some instances, methods comprise subjecting the blood to vertical filtration. In some instances, methods comprise subjecting the blood to a sample purifier comprising a filter matrix for receiving whole blood, the filter matrix having a pore size that is prohibitive for cells to pass through, while plasma can pass through the filter matrix uninhibited. Such vertical filtration and filter matrices are described for devices disclosed herein.

In some instances, isolating or purifying comprises subjecting a biological sample, or a fraction thereof, or a modified version thereof, to a binding moiety. The binding moiety may be capable of binding to a component of a biological sample and removing it to produce a modified sample depleted of cells, cell fragments, nucleic acids or proteins that are unwanted or of no interest. In some instances, isolating or purifying comprises subjecting a biological sample to a binding moiety to reduce unwanted substances or non-nucleic acid components in a biological sample. In some instances, isolating or purifying comprises subjecting a biological sample to a binding moiety to produce a modified sample enriched with target cell, target cell fragments, target nucleic acids or target proteins. By way of non-limiting example, isolating or purifying may comprise subjecting a biological sample to a binding moiety for capturing placenta educated platelets, which may contain fetal DNA or RNA fragments. The resulting cell-bound binding moieties can be captured/enriched for with antibodies or other methods, e.g., low speed centrifugation.

Isolating or purifying may comprise capturing an extracellular vesicle or extracellular microparticle in the biological sample with a binding moiety. In some instances, the extracellular vesicle contains at least one of DNA and RNA. In some instances, the extracellular vesicle is fetal/placental in origin. Methods may comprise capturing an extracellular vesicle or extracellular microparticle in the biological sample that comes from a maternal cell. In some instances, methods disclosed herein comprise capturing and discarding an extracellular vesicle or extracellular microparticle from a maternal cell to enrich the sample for fetal/placental nucleic acids.

In some instances, methods comprise capturing a nucleosome in a biological sample and analyzing nucleic acids attached to the nucleosome. In some instances, methods comprise capturing an exosome in a biological sample and analyzing nucleic acids attached to the exosome. Capturing nucleosomes and/or exosomes may preclude the need for a lysis step or reagent, thereby simplifying the method and reducing time from sample collection to detection.

In some instances, methods comprise subjecting a biological sample to a cell-binding moiety for capturing placenta educated platelets, which may contain fetal DNA or RNA fragments. Capturing may comprise contacting the placenta educated platelets with a binding moiety (e.g., an antibody for a cell surface marker), subjecting the biological sample to low speed centrifugation, or a combination thereof. In some instances, the binding moiety is attached to a solid support disclosed herein, and methods comprise separating the solid support from the rest of the biological sample after the binding moiety has made contact with the biological sample.

In some instances, isolating or purifying comprises reducing unwanted non-nucleic acid components from a biological sample. In some instances, isolating or purifying comprises removing unwanted non-nucleic acid components from a biological sample. In some instances, isolating or purifying comprises removing at least 5%, at least 10%, at least 20%, at least 30%, at least 40%, at least 50%, at least 60%, at least 70%, at least 80%, or at least 90% of unwanted non-nucleic acid components from a biological sample. In some instances, isolating or purifying comprises removing at least 95% of unwanted non-nucleic acid components from a biological sample. In some instances, isolating or purifying comprises removing at least 97% of unwanted non-nucleic acid components from a biological sample. In some instances, isolating or purifying comprises removing at least 98% of unwanted non-nucleic acid components from a biological sample. In some instances, isolating or purifying comprises removing at least 99% of unwanted non-nucleic acid components from a biological sample.

In some instances, methods disclosed herein comprise purifying nucleic acids in a sample. In some instances, purifying comprises washing the nucleic acids with a wash buffer. In some instances, purifying does not comprise washing the nucleic acids with a wash buffer. In some embodiments, purifying comprises capturing the nucleic acids with a nucleic acid capturing moiety to produce captured nucleic acids. Non-limiting examples of nucleic acid capturing moieties are silica particles and paramagnetic particles. In some embodiments, purifying comprises passing the sample containing the captured nucleic acids through a hydrophobic phase (e.g., a liquid or wax). The hydrophobic phase retains impurities in the sample that would otherwise inhibit further manipulation (e.g., amplification, sequencing) of the nucleic acids.

In some instances, methods disclosed herein comprise removing nucleic acid components from a biological sample described herein. In some instances, the removed nucleic acid components are discarded. By way of non-limiting example, methods may comprise analyzing only DNA. Thus, RNA is unwanted and creates undesirable background noise or contamination to the DNA. In some instances, methods disclosed herein comprise removing RNA from a biological sample. In some instances, methods disclosed herein comprise removing mRNA from a biological sample. In some instances, methods disclosed herein comprise removing microRNA from a biological sample. In some instances, methods disclosed herein comprise removing maternal RNA from a biological sample. In some instances, methods disclosed herein comprise removing DNA from a biological sample. In some instances, methods disclosed herein comprise removing maternal DNA from a biological sample of a pregnant subject. In some instances, removing nucleic acid components comprises contacting the nucleic acid components with an oligonucleotide capable of hybridizing to the nucleic acid, wherein the oligonucleotide is conjugated, attached or bound to a capturing device (e.g., bead, column, matrix, nanoparticle, magnetic particle, etc.).

In some instances, removing nucleic acid components comprises separating the nucleic acid components on a gel by size. For example, circulating cell free fetal DNA fragments are smaller than circulating maternal DNA fragments. Circulating cell free fetal DNA fragments are generally less than 200 base pairs in length. In some instances, methods disclosed herein comprise removing cell free DNA from the biological sample, wherein the cell free DNA has a minimum length. In some instances, the minimum length is about 50 base pairs. In some instances, the minimum length is about 100 base pairs. In some instances, the minimum length is about 110 base pairs. In some instances, the minimum length is about 120 base pairs. In some instances, the minimum length is about 140 base pairs. In some instances, methods disclosed herein comprise selecting cell free DNA from the biological sample, wherein the cell free DNA has a maximum length. In some instances, the maximum length is about 180 base pairs. In some instances, the maximum length is about 200 base pairs. In some instances, the maximum length is about 220 base pairs. In some instances, the maximum length is about 240 base pairs. In some instances, the maximum length is about 300 base pairs. In some instances, the maximum length is about 400 base pairs. In some instances, the maximum length is about 500 base pairs. Size based separation would be useful for other categories of nucleic acids having limited size ranges, which are well known in the art (e.g., microRNAs).

### Amplifying Nucleic Acids

In some instances, methods disclosed herein comprise amplifying at least one nucleic acid in a sample to produce at least one amplification product. The at least one nucleic acid may be a cell-free nucleic acid. The sample may be a biological sample disclosed herein or a fraction or portion thereof. The sample may be an environmental sample. In some instances, methods comprise producing a copy of the nucleic acid in the sample and amplifying the copy to produce the at least one amplification product. In some instances, methods comprise producing a reverse transcript of the nucleic acid in the sample and amplifying the reverse transcript to produce the at least one amplification product.

In some instances, methods disclosed herein comprise detecting a circulating cell free nucleic acid comprising a sequence of interest. In some instances, methods disclosed herein comprise quantifying a circulating cell free nucleic acid comprising a sequence of interest. In some instances, methods disclosed herein comprise amplifying a circulating cell free nucleic acid comprising a sequence of interest. In some instances, amplifying comprises polymerase mediated amplification with primers that anneal to a sense strand and antisense strand corresponding to a sequence of interest. In some instances, detecting or quantifying comprises hybridizing a circulating cell free nucleic acid comprising a sequence of interest to an oligonucleotide probe. The oligonucleotide probe may anneal to at least a portion of the sequence of interest or a complement thereof. By way of non-limiting example, the sequence of interest may be a sequence of a repetitive region (e.g., multiple copies of the sequence of interest) or a sequence specific to a Y chromosome.

In some instances, methods disclosed herein comprise amplifying a nucleic acid at least at one temperature. In some instances, methods disclosed herein comprise amplifying a nucleic acid at a single temperature (e.g., isothermal amplification). In some instances, methods disclosed herein comprise amplifying a nucleic acid, wherein the amplifying occurs at not more than two temperatures. Amplifying may occur in one step or multiple steps. Non-limiting examples of amplifying steps include double strand denaturing, primer hybridization, and primer extension.

In some instances, at least one step of amplifying occurs at room temperature. In some instances, all steps of amplifying occur at room temperature. In some instances, at least one step of amplifying occurs in a temperature range. In some instances, all steps of amplifying occur in a temperature range. In some instances, the temperature range is about 0° C. to about 100° C. In some instances, the temperature range is about 15° C. to about 100° C. In some instances, the temperature range is about 25° C. to about 100° C. In some instances, the temperature range is about 35° C. to about 100° C. In some instances, the temperature range is about 55° C. to about 100° C. In some instances, the temperature range is about 65° C. to about 100° C. In some instances, the temperature range is about 15° C. to about 80° C. In some instances, the temperature range is about 25° C. to about 80° C. In some instances, the temperature range is about 35° C. to about 80° C. In some instances, the temperature range is about 55° C. to about 80° C. In some instances, the temperature range is about 65° C. to about 80° C. In some instances, the temperature range is about 15° C. to about 60° C. In some instances, the temperature range is about 25° C. to about 60° C. In some instances, the temperature range is about 35° C. to about 60° C. In some instances, the temperature range is about 15° C. to about 40° C. In some instances, the temperature range is about −20° C. to about 100° C. In some instances, the temperature range is about −20° C. to about 90° C. In some instances, the temperature range is about −20° C. to about 50° C. In some instances, the temperature range is about −20° C. to about 40° C. In some instances, the temperature range is about −20° C. to about 10° C. In some instances, the temperature range is about 0° C. to about 100° C. In some instances, the temperature range is about 0° C. to about 40° C. In some instances, the temperature range is about 0° C. to about 30° C. In some instances, the temperature range is about 0° C. to about 20° C. In some instances, the temperature range is about 0° C. to about 10° C. In some instances, the temperature range is about 15° C. to about 100° C. In some instances, the temperature range is about 15° C. to about 90° C. In some instances, the temperature range is about 15° C. to about 80° C. In some instances, the temperature range is about is about 15° C. to about 70° C. In some instances, the temperature range is about 15° C. to about 60° C. In some instances, the temperature range is about 15° C. to about 50° C. In some instances, the temperature range is about 15° C. to about 30° C. In some instances, the temperature range is about 10° C. to about 30° C. In some instances, methods disclose herein are performed at room temperature, not requiring cooling, freezing or heating.

In some instances, amplifying a nucleic acid comprises contacting a nucleic acid with random oligonucleotide primers. Amplifying with a plurality of random primers generally results in non-targeted amplification of multiple nucleic acids of different sequences or an overall amplification of most nucleic acids in a sample. In some instances, amplifying comprises contacting cell free nucleic acid molecules disclosed herein with random oligonucleotide primers. In some instances, amplifying comprises contacting cell free fetal nucleic acid molecules disclosed herein with random oligonucleotide primers. In some instances, amplifying comprises contacting a tagged nucleic acid molecule disclosed herein with random oligonucleotide primers.

In some instances, amplifying comprises targeted amplification (e.g., selector method (described in U.S. Pat. No. 6,558,928), molecular inversion probes). In some instances, amplifying a nucleic acid comprises contacting a nucleic acid with at least one primer having a sequence corresponding to a target chromosome sequence. Exemplary chromosome sequences are disclosed herein. In some instances, amplifying comprises contacting the nucleic acid with at least one primer having a sequence corresponding to a non-target chromosome sequence. In some instances, amplifying comprises contacting the nucleic acid with not more than one pair of primers, wherein each primer of the pair of primers comprises a sequence corresponding to a sequence on a target chromosome disclosed herein. In some instances, amplifying comprises contacting the nucleic acid with multiple sets of primers, wherein each of a first pair in a first set and each of a pair in a second set are all different.

In some instances, amplifying comprises multiplexing (nucleic acid amplification of a plurality of nucleic acids in one reaction). In some instances, multiplexing comprises contacting nucleic acids of the biological sample with a plurality of oligonucleotide primer pairs. In some instances, multiplexing comprising contacting a first nucleic acid and a second nucleic acid, wherein the first nucleic acid corresponds to a first sequence and the second nucleic acid corresponds to a second sequence. In some instances, the first sequence and the second sequence are the same. In some instances, the first sequence and the second sequence are different. In some instances, amplifying does not comprise multiplexing. In some instances, amplifying does not require multiplexing. In some instance, amplifying comprises nested primer amplification.

In some instances, methods comprise amplifying a nucleic acid in the sample, wherein amplifying comprises contacting the sample with at least one oligonucleotide primer, wherein the at least one oligonucleotide primer is not active or extendable until it is in contact with the sample. In some instances, amplifying comprises contacting the sample with at least one oligonucleotide primer, wherein the at least one oligonucleotide primer is not active or extendable until it is exposed to a selected temperature. In some instances, amplifying comprises contacting the sample with at least one oligonucleotide primer, wherein the at least one oligonucleotide primer is not active or extendable until it is contacted with an activating reagent. By way of non-limiting example, the at least one oligonucleotide primer may comprise a blocking group. Using such oligonucleotide primers may minimize primer dimers, allow recognition of unused primer, and/or avoid false results caused by unused primers. In some instances, amplifying comprises contacting the sample with at least one oligonucleotide primer comprising a sequence corresponding to a sequence on a target chromosome disclosed herein.

In some instances, amplifying comprises the use of an oligonucleotide primer and one or more tags. The use of one or more tags may increase at least one of the efficiency, speed and accuracy of methods disclosed herein. In some instances, the oligonucleotide primer comprises a tag. In some instances, the tag comprises a nucleotide and the oligonucleotide primer comprises the tag. In some instances, the oligonucleotide primer is attached to a tag. In some instances, the oligonucleotide primer is conjugated to a tag. The tag may comprise an oligonucleotide, a small molecule, a peptide, or a combination thereof. In some instances, the tag comprises a nucleotide. In some instances, the tag does not comprise an oligonucleotide. In some instances, the tag comprises an amino acid. In some instances, the tag does not comprise an amino acid. In some instances, the tag comprises a peptide. In some instances, the tag does not comprise a peptide. In some instances, the tag is not sequence specific. In some instances, the tag comprises a polynucleotide having a generic sequence that does not correspond to any particular target sequence. In some instances, the tag is detectable when an amplification product is produced, regardless of the sequence amplified. In some instances, at least one of the oligonucleotide primer and tag comprises a peptide nucleic acid (PNA). In some instances, at least one of the oligonucleotide primer and tag comprises a locked nucleic acid (LNA).

In some instances, the oligonucleotide primer comprises an oligonucleotide tag having a sequence that is not specific to a sequence on the Y chromosome. Such a tag may be referred to as a universal tag. In other instances, wherein a target sequence or sequence of interest corresponds to a chromosome other than the Y chromosome, the tag can be specific to a sequence on the Y chromosome. In some instances, the tag is specific to a sequence other than the sequence of interest, but corresponds to the same chromosome as the sequence of interest. In some instances, the tag that is not specific to a sequence on a human chromosome. Alternatively or additionally, the oligonucleotide primer comprises an oligonucleotide tag having a sequence that is specific to a sequence on the Y chromosome. In some instances, methods comprise contacting the sample with a tag and at least one oligonucleotide primer comprising a sequence corresponding to a sequence on the Y chromosome, wherein the tag is separate from the oligonucleotide primer. In some instances, the tag is incorporated in an amplification product produced by extension of the oligonucleotide primer after it hybridizes to the Y chromosome fragment.

In some instances, amplifying comprises contacting the sample with at least one primer having a sequence corresponding to a sequence on the Y chromosome. In some instances, amplifying comprises contacting the sample with at least one primer having a sequence that is complementary to a sequence on the Y chromosome. In some instances, amplifying comprises contacting the sample with at least one primer having a sequence that is identical to a sequence on the Y chromosome. In some instances, amplifying comprises contacting the sample with at least one primer having a sequence that is at least 90% identical to a sequence on the Y chromosome. In some instances, amplifying comprises contacting the sample with at least one primer having a sequence that is at least 75% identical to a sequence on the Y chromosome. In some instances, amplifying comprises contacting the sample with at least one primer having a sequence that is at least 60% identical to a sequence on the Y chromosome. In some instances, amplifying comprises contacting the sample with not more than one pair of primers, wherein each primer of the pair of primers comprises a sequence corresponding to a sequence on the Y chromosome.

In some instances, methods disclosed herein comprise the use of a plurality of tags, thereby increasing at least one of the accuracy of the method, speed of the method and information obtained by the method. In some instances, methods disclosed herein comprise the use of a plurality of tags, thereby decreasing the volume of sample required to obtain a reliable result. In some instances, the plurality of tags comprises at least one capture tag. In some instances, the plurality of tags comprises at least one detection tag. A capture tag is generally used to isolate or separate a specific sequence or region from other regions. A typical example for a capture tag is biotin (that can be captured using streptavidin coated surfaces for example). Examples of detection tags are digoxigenin and a fluorescent tag. The detection tag may be detected directly (e.g., laser irradiation and/or measuring emitted light) or indirectly through an antibody that carries or interacts with a secondary detection system such as a luminescent assay or enzymatic assay. In some instances, the plurality of tags comprises a combination of least one capture tag (a tag used to isolate an analyte) and at least one detection tag (a tag used to detect the analyte). In some instance, a single tag acts as a detection tag and a capture tag.

In some instances, methods comprise contacting the at least one circulating cell free nucleic acid in the sample with a first tag and a second tag, wherein the first tag comprises a first oligonucleotide that is complementary to a sense strand of the circulating cell free nucleic acid, and the second capture tag comprises a second oligonucleotide that is complementary to an antisense strand of the circulating cell free nucleic acid. In some instances, methods comprise contacting the at least one circulating cell free nucleic acid in the sample with a first tag and a second tag, wherein the first tag carries the same label as the second tag. In some instances, methods comprise contacting the at least one circulating cell free nucleic acid in the sample with a first tag and a second tag, wherein the first tag carries a different label than the second tag. In some instances, the tags are the same and there is a single qualitative or quantitative signal that is the aggregate of all probes/regions detected. In some instances, the tags are different. One tag may be used to purify and one tag may be used to detect. In some instances, a first oligonucleotide tag is specific to a region (e.g., cfDNA fragment) and carries a fluorescent label and a second oligonucleotide is specific to an adjacent region and carries the same fluorescent label because only the aggregate signal is desired. In other instances, a first oligonucleotide tag is specific to a region (e.g., cfDNA fragment) and carries a fluorescent label and a second oligonucleotide is specific to an adjacent region and carries a different fluorescent label to detect two distinct regions.

Any appropriate nucleic acid amplification method known in the art is contemplated for use in the devices and methods described herein. In some instances, isothermal amplification is used. In some instances, amplification is isothermal with the exception of an initial heating step before isothermal amplification begins. A number of isothermal amplification methods, each having different considerations and providing different advantages, are known in the art and have been discussed in the literature, e.g., by Zanoli and Spoto, 2013, “Isothermal Amplification Methods for the Detection of Nucleic Acids in Microfluidic Devices,” Biosensors 3: 18-43, and Fakruddin, et al., 2013, “Alternative Methods of Polymerase Chain Reaction (PCR),” Journal of Pharmacy and Bioallied Sciences 5(4): 245-252, each incorporated herein by reference in its entirety. In some instances, any appropriate isothermic amplification method is used. In some instances, the isothermic amplification method used is selected from: Loop Mediated Isothermal Amplification (LAMP); Nucleic Acid Sequence Based Amplification (NASBA); Multiple Displacement Amplification (MDA); Rolling Circle Amplification (RCA); Helicase Dependent Amplification (HDA); Strand Displacement Amplification (SDA); Nicking Enzyme Amplification Reaction (NEAR); Ramification Amplification Method (RAM); and Recombinase Polymerase Amplification (RPA).

In some instances, the amplification method used is LAMP (see, e.g., Notomi, et al., 2000, “Loop Mediated Isothermal Amplification” NAR 28(12): e63 i-vii, and U.S. Pat. No. 6,410,278, “Process for synthesizing nucleic acid” each incorporated by reference herein in its entirety). LAMP is a one-step amplification system using auto-cycling strand displacement deoxyribonucleic acid (DNA) synthesis. In some instances, LAMP is carried out at 60-65° C. for 45-60 min in the presence of a thermostable polymerase, e.g., Bacillus stearothermophilus (Bst) DNA polymerase I, deoxyribonucleotide triphosphate (dNTPs), specific primers and the target DNA template. In some instances, the template is RNA and a polymerase having both reverse transcriptase activity and strand displacement-type DNA polymerase activity, e.g., Bca DNA polymerase, is used, or a polymerase having reverse transcriptase activity is used for the reverse transcriptase step and a polymerase not having reverse transcriptase activity is used for the strand displacement-DNA synthesis step.

In some instances, the amplification reaction is carried out using LAMP, at about 55° C. to about 70° C. In some instances, the LAMP reaction is carried out at 55° C. or greater. In some instances, the LAMP reaction is carried out 70° C. or less. In some instances, the LAMP reaction is carried out at about 55° C. to about 57° C., about 55° C. to about 59° C., about 55° C. to about 60° C., about 55° C. to about 61° C., about 55° C. to about 62° C., about 55° C. to about 63° C., about 55° C. to about 64° C., about 55° C. to about 65° C., about 55° C. to about 66° C., about 55° C. to about 68° C., about 55° C. to about 70° C., about 57° C. to about 59° C., about 57° C. to about 60° C., about 57° C. to about 61° C., about 57° C. to about 62° C., about 57° C. to about 63° C., about 57° C. to about 64° C., about 57° C. to about 65° C., about 57° C. to about 66° C., about 57° C. to about 68° C., about 57° C. to about 70° C., about 59° C. to about 60° C., about 59° C. to about 61° C., about 59° C. to about 62° C., about 59° C. to about 63° C., about 59° C. to about 64° C., about 59° C. to about 65° C., about 59° C. to about 66° C., about 59° C. to about 68° C., about 59° C. to about 70° C., about 60° C. to about 61° C., about 60° C. to about 62° C., about 60° C. to about 63° C., about 60° C. to about 64° C., about 60° C. to about 65° C., about 60° C. to about 66° C., about 60° C. to about 68° C., about 60° C. to about 70° C., about 61° C. to about 62° C., about 61° C. to about 63° C., about 61° C. to about 64° C., about 61° C. to about 65° C., about 61° C. to about 66° C., about 61° C. to about 68° C., about 61° C. to about 70° C., about 62° C. to about 63° C., about 62° C. to about 64° C., about 62° C. to about 65° C., about 62° C. to about 66° C., about 62° C. to about 68° C., about 62° C. to about 70° C., about 63° C. to about 64° C., about 63° C. to about 65° C., about 63° C. to about 66° C., about 63° C. to about 68° C., about 63° C. to about 70° C., about 64° C. to about 65° C., about 64° C. to about 66° C., about 64° C. to about 68° C., about 64° C. to about 70° C., about 65° C. to about 66° C., about 65° C. to about 68° C., about 65° C. to about 70° C., about 66° C. to about 68° C., about 66° C. to about 70° C., or about 68° C. to about 70° C. In some instances, the LAMP reaction is carried out at about 55° C., about 57° C., about 59° C., about 60° C., about 61° C., about 62° C., about 63° C., about 64° C., about 65° C., about 66° C., about 68° C., or about 70° C.

In some instances, the amplification reaction is carried out using LAMP, for about 30 to about 90 minutes. In some instances, the LAMP reaction is carried out for at least about 30 minutes. In some instances, the LAMP reaction is carried out for at most about 90 minutes. In some instances, the LAMP reaction is carried out for about 30 minutes to about 35 minutes, about 30 minutes to about 40 minutes, about 30 minutes to about 45 minutes, about 30 minutes to about 50 minutes, about 30 minutes to about 55 minutes, about 30 minutes to about 60 minutes, about 30 minutes to about 65 minutes, about 30 minutes to about 70 minutes, about 30 minutes to about 75 minutes, about 30 minutes to about 80 minutes, about 30 minutes to about 90 minutes, about 35 minutes to about 40 minutes, about 35 minutes to about 45 minutes, about 35 minutes to about 50 minutes, about 35 minutes to about 55 minutes, about 35 minutes to about 60 minutes, about 35 minutes to about 65 minutes, about 35 minutes to about 70 minutes, about 35 minutes to about 75 minutes, about 35 minutes to about 80 minutes, about 35 minutes to about 90 minutes, about 40 minutes to about 45 minutes, about 40 minutes to about 50 minutes, about 40 minutes to about 55 minutes, about 40 minutes to about 60 minutes, about 40 minutes to about 65 minutes, about 40 minutes to about 70 minutes, about 40 minutes to about 75 minutes, about 40 minutes to about 80 minutes, about 40 minutes to about 90 minutes, about 45 minutes to about 50 minutes, about 45 minutes to about 55 minutes, about 45 minutes to about 60 minutes, about 45 minutes to about 65 minutes, about 45 minutes to about 70 minutes, about 45 minutes to about 75 minutes, about 45 minutes to about 80 minutes, about 45 minutes to about 90 minutes, about 50 minutes to about 55 minutes, about 50 minutes to about 60 minutes, about 50 minutes to about 65 minutes, about 50 minutes to about 70 minutes, about 50 minutes to about 75 minutes, about 50 minutes to about 80 minutes, about 50 minutes to about 90 minutes, about 55 minutes to about 60 minutes, about 55 minutes to about 65 minutes, about 55 minutes to about 70 minutes, about 55 minutes to about 75 minutes, about 55 minutes to about 80 minutes, about 55 minutes to about 90 minutes, about 60 minutes to about 65 minutes, about 60 minutes to about 70 minutes, about 60 minutes to about 75 minutes, about 60 minutes to about 80 minutes, about 60 minutes to about 90 minutes, about 65 minutes to about 70 minutes, about 65 minutes to about 75 minutes, about 65 minutes to about 80 minutes, about 65 minutes to about 90 minutes, about 70 minutes to about 75 minutes, about 70 minutes to about 80 minutes, about 70 minutes to about 90 minutes, about 75 minutes to about 80 minutes, about 75 minutes to about 90 minutes, or about 80 minutes to about 90 minutes. In some instances, the LAMP reaction is carried out for about 30 minutes, about 35 minutes, about 40 minutes, about 45 minutes, about 50 minutes, about 55 minutes, about 60 minutes, about 65 minutes, about 70 minutes, about 75 minutes, about 80 minutes, or about 90 minutes.

In some instances, the amplification method is Nucleic Acid Sequence Based Amplification (NASBA). NASBA (also known as 3 SR, and transcription-mediated amplification) is an isothermal transcription-based RNA amplification system. Three enzymes (avian myeloblastosis virus reverse transcriptase, RNase H and T7 DNA dependent RNA polymerase) are used to generate single-stranded RNA. In certain cases NASBA can be used to amplify DNA. The amplification reaction is performed at 41° C., maintaining constant temperature, typically for about 60 to about 90 minutes (see, e.g., Fakruddin, et al., 2012, “Nucleic Acid Sequence Based Amplification (NASBA) Prospects and Applications,” Int. J. of Life Science and Pharma Res. 2(1):L106-L121, incorporated by reference herein).

In some instances, the NASBA reaction is carried out at about 40° C. to about 42° C. In some instances, the NASBA reaction is carried out at 41° C. In some instances, the NASBA reaction is carried out at at most about 42° C. In some instances, the NASBA reaction is carried out at about 40° C. to about 41° C., about 40° C. to about 42° C., or about 41° C. to about 42° C. In some instances, the NASBA reaction is carried out at about 40° C., about 41° C., or about 42° C.

In some instances, the amplification reaction is carried out using NASBA, for about 45 to about 120 minutes. In some instances, the NASBA reaction is carried out for about 30 minutes to about 120 minutes. In some instances, the NASBA reaction is carried out for at least about 30 minutes. In some instances, the NASBA reaction is carried out for at most about 120 minutes. In some instances, the NASBA reaction is carried out for up to 180 minutes. In some instances, the NASBA reaction is carried out for about 30 minutes to about 45 minutes, about 30 minutes to about 60 minutes, about 30 minutes to about 65 minutes, about 30 minutes to about 70 minutes, about 30 minutes to about 75 minutes, about 30 minutes to about 80 minutes, about 30 minutes to about 85 minutes, about 30 minutes to about 90 minutes, about 30 minutes to about 95 minutes, about 30 minutes to about 100 minutes, about 30 minutes to about 120 minutes, about 45 minutes to about 60 minutes, about 45 minutes to about 65 minutes, about 45 minutes to about 70 minutes, about 45 minutes to about 75 minutes, about 45 minutes to about 80 minutes, about 45 minutes to about 85 minutes, about 45 minutes to about 90 minutes, about 45 minutes to about 95 minutes, about 45 minutes to about 100 minutes, about 45 minutes to about 120 minutes, about 60 minutes to about 65 minutes, about 60 minutes to about 70 minutes, about 60 minutes to about 75 minutes, about 60 minutes to about 80 minutes, about 60 minutes to about 85 minutes, about 60 minutes to about 90 minutes, about 60 minutes to about 95 minutes, about 60 minutes to about 100 minutes, about 60 minutes to about 120 minutes, about 65 minutes to about 70 minutes, about 65 minutes to about 75 minutes, about 65 minutes to about 80 minutes, about 65 minutes to about 85 minutes, about 65 minutes to about 90 minutes, about 65 minutes to about 95 minutes, about 65 minutes to about 100 minutes, about 65 minutes to about 120 minutes, about 70 minutes to about 75 minutes, about 70 minutes to about 80 minutes, about 70 minutes to about 85 minutes, about 70 minutes to about 90 minutes, about 70 minutes to about 95 minutes, about 70 minutes to about 100 minutes, about 70 minutes to about 120 minutes, about 75 minutes to about 80 minutes, about 75 minutes to about 85 minutes, about 75 minutes to about 90 minutes, about 75 minutes to about 95 minutes, about 75 minutes to about 100 minutes, about 75 minutes to about 120 minutes, about 80 minutes to about 85 minutes, about 80 minutes to about 90 minutes, about 80 minutes to about 95 minutes, about 80 minutes to about 100 minutes, about 80 minutes to about 120 minutes, about 85 minutes to about 90 minutes, about 85 minutes to about 95 minutes, about 85 minutes to about 100 minutes, about 85 minutes to about 120 minutes, about 90 minutes to about 95 minutes, about 90 minutes to about 100 minutes, about 90 minutes to about 120 minutes, about 95 minutes to about 100 minutes, about 95 minutes to about 120 minutes, or about 100 minutes to about 120 minutes. In some instances, the NASBA reaction is carried out for about 30 minutes, about 45 minutes, about 60 minutes, about 65 minutes, about 70 minutes, about 75 minutes, about 80 minutes, about 85 minutes, about 90 minutes, about 95 minutes, about 100 minutes, about 120 minutes, about 150 minutes, or about 180 minutes.

In some instances, the amplification method is Strand Displacement Amplification (SDA). SDA is an isothermal amplification method that uses four different primers. A primer containing a restriction site (a recognition sequence for HincII exonuclease) is annealed to the DNA template. An exonuclease-deficient fragment of Eschericia coli DNA polymerase 1 (exo-Klenow) elongates the primers. Each SDA cycle consists of (1) primer binding to a displaced target fragment, (2) extension of the primer/target complex by exo-Klenow, (3) nicking of the resultant hemiphosphothioate HincII site, (4) dissociation of HincII from the nicked site and (5) extension of the nick and displacement of the downstream strand by exo-Klenow.

In some instances, methods comprise contacting DNA in a sample with a helicase. In some instances, the amplification method is Helicase Dependent Amplification (HDA). HDA is an isothermal reaction because a helicase, instead of heat, is used to denature DNA.

In some instances, the amplification method is Multiple Displacement Amplification (MDA). The MDA is an isothermal, strand-displacing method based on the use of the highly processive and strand-displacing DNA polymerase from bacteriophage Ø29, in conjunction with modified random primers to amplify the entire genome with high fidelity. It has been developed to amplify all DNA in a sample from a very small amount of starting material. In MDA Ø29 DNA polymerase is incubated with dNTPs, random hexamers and denatured template DNA at 30° C. for 16 to 18 hours and the enzyme must be inactivated at high temperature (65° C.) for 10 min. No repeated recycling is required, but a short initial denaturation step, the amplification step, and a final inactivation of the enzyme are needed.

In some instances, the amplification method is Rolling Circle Amplification (RCA). RCA is an isothermal nucleic acid amplification method which allows amplification of the probe DNA sequences by more than 10⁹ fold at a single temperature, typically about 30° C. Numerous rounds of isothermal enzymatic synthesis are carried out by Ø29 DNA polymerase, which extends a circle-hybridized primer by continuously progressing around the circular DNA probe. In some instances, the amplification reaction is carried out using RCA, at about 28° C. to about 32° C.

Additional amplification methods can be found in the art that could be incorporated into devices and methods disclosed herein. Ideally, the amplification method is isothermal and fast relative to traditional PCR. In some instances, amplifying comprises performing an exponential amplification reaction (EXPAR), which is an isothermal molecular chain reaction in that the products of one reaction catalyze further reactions that create the same products. In some instances, amplifying occurs in the presence of an endonuclease. The endonuclease may be a nicking endonuclease. See, e.g., Wu et al., “Aligner-Mediated Cleavage of Nucleic Acids,” Chemical Science (2018). In some instances, amplifying does not require initial heat denaturation of target DNA. See, e.g., Toley et al., “Isothermal strand displacement amplification (iSDA): a rapid and sensitive method of nucleic acid amplification for point-of-care diagnosis,” The Analyst (2015). Pulse controlled amplification in an ultrafast amplification method developed by GNA Biosolutions GmbH.

### Sequencing

In some instances, methods disclosed herein comprise sequencing a nucleic acid. The nucleic acid may be a nucleic acid disclosed herein, such as a tagged nucleic acid, an amplified nucleic acid, a cell-free nucleic acid, a cell-free fetal nucleic acid, a nucleic acid having a sequence corresponding to a target chromosome, a nucleic acid having a sequence corresponding to a region of a target chromosome, a nucleic acid having a sequence corresponding to a non-target chromosome, or a combination thereof. In some instances, the nucleic acid is DNA. In some instances, the nucleic acid is RNA. In some instances, the nucleic acid comprises DNA. In some instances, the nucleic acid comprises RNA. In some instances, methods comprise bisulfite sequencing to detect epigenetic modifications.

In some instances, sequencing comprises targeted sequencing. In some instances, sequencing comprises whole genome sequencing. In some instances, sequencing comprises targeted sequencing and whole genome sequencing. In some instances, whole genome sequencing comprises massive parallel sequencing, also referred to in the art as next generation sequencing or second generation sequencing. In some instances, whole genome sequencing comprises random massive parallel sequencing. In some instances, sequencing comprises random massive parallel sequencing of target regions captured from a whole genome library.

In some instances, methods comprise sequencing amplified nucleic acids disclosed herein. In some instances, amplified nucleic acids are produced by targeted amplification (e.g., with primers specific to target sequences of interest). In some instances, amplified nucleic acids are produced by non-targeted amplification (e.g., with random oligonucleotide primers). In some instances, methods comprise sequencing amplified nucleic acids, wherein the sequencing comprises massive parallel sequencing.

### Library Preparation

In some instances, methods disclosed herein comprise modifying nucleic acids in the biological sample to produce a library of nucleic acids for detection. In some instances, methods comprise modifying nucleic acids for nucleic acid sequencing. In some instances, methods comprise modifying nucleic acids for detection, wherein detection does not comprise nucleic acid sequencing. In some instances, methods comprise modifying nucleic acids for detection, wherein detection comprises counting tagged nucleic acids based on an occurrence of tag detection. In some instances, methods disclosed herein comprise modifying nucleic acids in the biological sample to produce a library of nucleic acids, wherein the method comprises amplifying the nucleic acids. In some instances, modifying occurs before amplifying. In some instances, modifying occurs after amplifying.

In some instances, methods disclosed herein comprise preparing a non-selective library (e.g., all or many available cfDNA or DNA analyte fragments get incorporated in library preparation). In other instances, methods disclosed herein comprise preparing a targeted library or selective library where nucleic acids of interest are selected prior to or during the library preparation. By way of non-limiting example, one could prepare a Y chromosome specific library (e.g., DNA fragments having sequences found only on the Y chromosome). Similarly, one could prepare an X chromosome specific library, autosome specific library or a custom library with specific sequences, genes, or gene regions of interest.

In some instances, modifying the nucleic acids comprises repairing ends of nucleic acids that are fragments of a nucleic acid. By way of non-limiting example, repairing ends may comprise restoring a 5′ phosphate group, a 3′ hydroxy group, or a combination thereof to the nucleic acid. In some instances, repairing may comprise removing overhangs. In some instances, repairing may comprise filling in overhangs with complementary nucleotides.

In some instances, modifying the nucleic acids for preparing a library comprises use of an adapter. The adapter may also be referred to herein as a sequencing adapter. In some instances, the adapter aids in sequencing. Generally, the adapter comprises an oligonucleotide. By way of non-limiting example, the adapter may simplify other steps in the methods, such as amplifying, purification and sequencing because it is a sequence that is universal to multiple, if not all, nucleic acids in a sample after modifying. In some instances, modifying the nucleic acids comprises ligating an adapter to the nucleic acids. Ligating may comprise blunt ligation. In some instances, modifying the nucleic acids comprises hybridizing an adapter to the nucleic acids.

In some instances, modifying the nucleic acids for preparing a library comprises use of a tag. The tag may also be referred to herein as a barcode. In some instances, methods disclosed herein comprise modifying nucleic acids with a tag that corresponds to a chromosomal region of interest. In some instances, methods disclosed herein comprise modifying nucleic acids with a tag that is specific to a chromosomal region that is not of interest. In some instances, methods disclosed herein comprise modifying a first portion of nucleic acids with a first tag that corresponds to at least one chromosomal region that is of interest and a second portion of nucleic acids with a second tag that corresponds to at least one chromosomal region that is not of interest. In some instances, modifying the nucleic acids comprises ligating a tag to the nucleic acids. Ligating may comprise blunt ligation. In some instances, modifying the nucleic acids comprises hybridizing a tag to the nucleic acids. In some instances, the tags comprise oligonucleotides. In some instances, the tags comprise a non-oligonucleotide marker or label that can be detected by means other than nucleic acid analysis. By way of non-limiting example, a non-oligonucleotide marker or label could comprise a fluorescent molecule, a nanoparticle, a dye, a peptide, or other detectable/quantifiable small molecule.

In some instances, modifying the nucleic acids for preparing a library comprises use of a sample index, also simply referred to herein as an index. By way of non-limiting example, the index may comprise an oligonucleotide, a small molecule, a nanoparticle, a peptide, a fluorescent molecule, a dye, or other detectable/quantifiable moiety. In some instances, a first group of nucleic acids from a first biological sample are labeled with a first index, and a first group of nucleic acids from a first biological sample are labeled with a second index, wherein the first index and the second index are different. Thus, multiple indexes allow for distinguishing nucleic acids from multiple samples when multiple samples are analyzed at once. In some instances, methods disclose amplifying nucleic acids wherein an oligonucleotide primer used to amplify the nucleic acids comprises an index.

In some instances, methods comprise detecting an amplification product, wherein the amplification product is produced by amplifying at least a portion of a target chromosome disclosed herein, or fragment thereof. The portion or fragment of the target chromosome may comprise at least 5 nucleotides. The portion or fragment of the target chromosome may comprise at least about 10 nucleotides. The portion or fragment of the target chromosome may comprise at least about 15 nucleotides. In some instances, detecting amplification products disclosed herein does not comprise tagging or labeling the amplification product. In some instances, methods detect the amplification product based on its amount. For example, the methods may detect an increase in the amount of double stranded DNA in the sample. In some instances, detecting the amplification product is at least partially based on its size. In some instances, the amplification product has a length of about 50 base pairs to about 500 base pairs.

In some instances, detecting the amplification product comprises contacting the amplification product with a tag. In some instances, the tag comprises a sequence that is complementary to a sequence of the amplification product. In some instances, the tag does not comprise a sequence that is complementary to a sequence of the amplification product. Non-limiting examples of tags are described in the foregoing and following disclosure.

In some instances, detecting the amplification product, whether tagged or not tagged, comprises subjecting the amplification product to a signal detector or assay assembly of a device, system, or kit disclosed herein. In some instances, methods comprise comprises amplifying and detecting on an assay assembly of a device, system, or kit disclosed herein. In some instances, the assay assembly comprises amplification reagents.

In some aspects, disclosed herein are methods comprising: obtaining a fluid sample from a female pregnant subject; contacting at least one circulating cell free nucleic acid in the sample with at least one tag to produce a tagged nucleic acid, wherein the circulating cell free nucleic acid comprises a sequence corresponding to a Y chromosome; and detecting the tagged nucleic acid. In some instances, methods further comprise amplifying the tagged nucleic acid to produce a plurality of tagged nucleic acids and detecting the plurality of tagged nucleic acids. In some instances, the tag enables capture of the circulating cell free nucleic acid or an amplification product thereof. In some instances, the tag enables detection of the circulating cell free nucleic acid or an amplification product thereof. In some instances, the circulating cell free nucleic acid is double stranded DNA and the methods comprise separating at least a portion of the double stranded DNA to produce single stranded DNA before contacting the at least one circulating cell free nucleic acid in the sample with at least one tag. In some instances, separating comprises applying heat to the cell free nucleic acid. In some instances, separating comprises applying an enzyme to the cell free nucleic acid. In some instances, the tag comprises an oligonucleotide. In some instances, the tag comprises a peptide or protein. In some instances, the tag comprises a small molecule. The small molecule may be organic or inorganic.

In some instances, methods disclosed herein comprise contacting at least one nucleic acid in the biological sample with a tagged oligonucleotide primer. In some instances, the tagged oligonucleotide primer comprises an oligonucleotide primer and an oligonucleotide tag. In some instances, the tagged oligonucleotide primer comprises an oligonucleotide primer and a tag, wherein the tag does not comprise a nucleotide. In some instances, the tagged oligonucleotide primer comprises an oligonucleotide primer and a tag, wherein the tag does not comprise an oligonucleotide. In some instances, the tagged oligonucleotide primer comprises an oligonucleotide primer and a peptide tag. In some instances, the tagged oligonucleotide primer comprises an oligonucleotide primer and a small molecule tag. In some aspects, disclosed herein are methods comprising: obtaining a fluid sample from a female pregnant subject; contacting at least one circulating cell free nucleic acid in the sample with at least one tagged oligonucleotide primer, wherein the circulating cell free nucleic acid comprises a sequence corresponding to a Y chromosome; amplifying the circulating cell free nucleic acid by contacting the extending the circulating cell free nucleic acid with a polymerase and free nucleotides to produce a tagged amplification product; and detecting the tag portion of the tagged amplification product. In some instances, the circulating cell free nucleic acid is double stranded DNA and the methods comprise separating at least a portion of the double stranded DNA to produce single stranded DNA before contacting the at least one circulating cell free nucleic acid in the sample with the at least one tagged oligonucleotide primer. In some instances, separating comprises applying heat to the cell free nucleic acid. In some instances, separating comprises applying an enzyme to the cell free nucleic acid.

In some instances, the tagged oligonucleotide primer comprises an oligonucleotide tag, wherein the oligonucleotide tag does not correspond to a sequence on the Y chromosome. In some instances, methods comprise tagging a Y chromosome, or fragment thereof, in the sample with an oligonucleotide tag that is not specific to a sequence on the Y chromosome. In some instances, the oligonucleotide tag is not specific to a sequence on a human chromosome. Alternatively or additionally, methods comprise contacting the sample with an oligonucleotide tag and at least one oligonucleotide primer, wherein the oligonucleotide primer comprises a sequence corresponding to a sequence on the Y chromosome, wherein the oligonucleotide tag is separate from the oligonucleotide primer. In some instances, the oligonucleotide tag is incorporated in an amplification product produced by extension of the oligonucleotide primer after it hybridizes to the Y chromosome fragment. In some instances, the oligonucleotide tag is detectable when an amplification product is produced, regardless of the sequence amplified. In some instances, at least one of the oligonucleotide primer and oligonucleotide tag comprises a peptide nucleic acid (PNA). In some instances, the oligonucleotide tag comprises a locked nucleic acid (LNA).

In some instances, methods disclosed herein comprise the use of a plurality of tags, thereby increasing at least one of the accuracy of the method, speed of the method and information obtained by the method. In some instances, methods disclosed herein comprise the use of a plurality of tags, thereby decreasing the volume of sample required to obtain a reliable result. In some instances, methods disclosed herein comprise contacting nucleic acids in the biological sample with a plurality of tags to a plurality of regions of the Y chromosome. In some instances, methods disclosed herein comprise contacting nucleic acids in the biological sample with a plurality of tags to a plurality of regions of the Y chromosome, thereby tagging the whole Y chromosome. In some instances, methods disclosed herein comprise contacting nucleic acids in the biological sample with a plurality of tags to a plurality of regions of the Y chromosome, thereby tagging a percentage of the Y chromosome. In some instances, the percentage is about 1% to about 99%. In some instances, the percentage is about 10% to 99%. In some instances, the percentage is about 10% to about 99%. In some instances, the percentage is about 20% to 99%. In some instances, the percentage is about 30% to about 99%. In some instances, the percentage is about 40% to about 99%. In some instances, the percentage is about 50% to about 99%. In some instances, the percentage is about 60% to about 99%. In some instances, the percentage is about 70% to about 99%. In some instances, the percentage is about 80% to about 99%. In some instances, the percentage is about 90% to about 99%. In some instances, the percentage is about 1% to about 99%. In some instances, the percentage is about 10% to about 20%. In some instances, the percentage is about 10% to about 30%. In some instances, the percentage is about 10% to about 40%. In some instances, the percentage is about 10% to about 50%. In some instances, the percentage is about 10% to about 60%. In some instances, the percentage is about 10% to about 70%. In some instances, the percentage is about 60% to about 99%. In some instances, the percentage is about 10% to about 80%. In some instances, the percentage is about 80% to about 99%. In some instances, the percentage is about 10% to about 90%.

In some instances, the plurality of tags comprises at least one capture tag. In some instances, the plurality of tags comprises at least one detection tag. In some instances, the plurality of tags comprises a combination of least one capture tag and at least one detection tag. In some instances, methods comprise contacting the at least one circulating cell free nucleic acid in the sample with a first tag and a second tag, wherein the first tag comprises a first oligonucleotide that is complementary to a sense strand of the circulating cell free nucleic acid, and the second capture tag comprises a second oligonucleotide that is complementary to an antisense strand of the circulating cell free nucleic acid. In some instances, methods comprise contacting the at least one circulating cell free nucleic acid in the sample with a first tag and a second tag, wherein the first tag carries the same label as the second tag. In some instances, methods comprise contacting the at least one circulating cell free nucleic acid in the sample with a first tag and a second tag, wherein the first tag carries a different label than the second tag.

### Detecting & Determining Genetic Information

In general, methods disclosed herein comprise detecting a biomarker, an analyte or a modified form thereof. Methods may comprise detecting a plurality of analytes that share a common feature. In some instances, the analytes are nucleic acids, the common feature is a sequence, and detecting comprises sequencing a nucleic acid or amplicon thereof. In some instances, the common feature is an epigenetic status such as a methylation status. In some instances, methods comprise detecting a tag or signal on a target analyte.

In some instances, methods comprise detecting nucleic acids. In some instances, methods comprise detecting cell-free nucleic acids. In some instances, methods comprise detecting a tag that has been ligated or hybridized to a nucleic acid. In some instances, methods comprise detecting an amplicon of a nucleic acid. Alternatively or additionally, methods comprise detecting a non-nucleic acid component. By way of non-limiting example, the non-nucleic acid component may be selected from a protein, a peptide, a lipid, a fatty acid, a sterol, a carbohydrate, a viral component, a microbial component, and a combination thereof. In the instance of a viral component or a microbial component, methods may comprise releasing, purifying, and/or amplifying a nucleic acid from a virus or bacteria before detecting.

Methods may comprise detecting a detectable label or detectable signal of a nucleic acid or non-nucleic acid component. Methods may comprise detecting a detectable label or detectable signal of a binding moiety (e.g., small molecule, peptide, aptamer, antibody, or antigen binding fragment thereof) that binds the nucleic acid or non-nucleic acid component. By way of non-limiting example, the detectable label or signal may be a fluorescent molecule, a bioluminescent molecule, a luminescent molecule, a radioactive signal, a magnetic signal, an electric signal, or a dye. For example, methods may comprise detecting an interaction between the binding moiety and a protein of interest. By way of non-limiting example, detecting may comprise performing IPCR or PLA.

Methods disclosed herein may comprise detecting and/or monitoring epigenetic changes from small amounts of a biological sample. Methods may comprise detecting the epigenetic status of multiple cell-free DNA fragments from one or more target regions. Methods may comprise detecting the epigenetic status of multiple cytosines from one or more target regions that are sufficiently distant from each other to be present on separate cell-free DNA fragments. By way of example, assessing cytosine methylation in circulating cell-free DNA from the INS1 gene locus can be indicative of B-cell degradation found in autoimmune Type 1 diabetes and therefore may serve as a biomarker of a risk for Type 1 diabetes. Similarly, the cytosine methylation status of genes encoding the myelin oligodendrocyte glycoprotein (MOG), myelin basic protein (MBP), Macroglobulinemia, Waldenstrom, Susceptibility To, 1 protein (WM1), or a combination thereof, can serve as a noninvasive biomarker for multiple sclerosis (MS). As a further example, assessing cytosine methylation in the CpG island of the promoter of the AOX1 gene can aid diagnosis of prostate cancer (PCa) and may allow to monitor progression and treatment success. The AOX1 gene is location on chromosome 2. The promoter CpG island is located between base positions 200,585,800 and 200,586,350, spanning about 500 bp. It contains at least 34 CpG nucleotides that are all hyper methylated in prostate cancer but not methylated in normal samples. Analysis of these specific CpG nucleotides in circulating cell-free DNA as a group, subgroups or at an individual level can be performed with devices, systems and methods described in this application.

Detecting may comprise amplifying, as described herein. For example, amplifying may comprise qPCR in which a signal is generated based on the presence or absence of a target analyte. In some instances, methods comprise detecting a nucleic acid amplification product from a LAMP reaction by detecting turbidity in the LAMP reaction vessel (dubbed a real-time turbidimeter by Mori et al. (2004) 59:145-147). LAMP produces large amounts of a specific amplicon quickly, simultaneously forming precipitates of magnesium pyrophosphate. These precipitates create turbidity that acts as a sign of successful amplification. Thus amplicons can be detected in real-time without actually probing for the amplicon or needing a separate detectable signal.

In some instances, methods comprise detecting an amplification product, wherein the amplification product is produced by amplifying at least a portion of a target region. The target region may comprise at least 5 nucleotides. The target region may comprise at least about 10 nucleotides. The target region may comprise at least about 15 nucleotides. In some instances, detecting amplification products disclosed herein does not comprise tagging or labeling the amplification product. In some instances, methods detect the amplification product based on its amount. For example, the methods may detect an increase in the amount of double stranded DNA in the sample. In some instances, detecting the amplification product is at least partially based on its size. In some instances, the amplification product has a length of about 50 base pairs to about 250 base pairs. In some instances, the amplification product has a length of about 50 base pairs to about 300 base pairs. In some instances, the amplification product has a length of about 50 base pairs to about 400 base pairs. In some instances, the amplification product has a length of about 50 base pairs to about 500 base pairs. In some instances, the amplification product has a length of about 50 base pairs to about 1000 base pairs.

In some instances, detecting an amplification product comprises contacting the amplification product with a tag. In some instances, the tag comprises a sequence that is complementary to a sequence of the amplification product. In some instances, the tag does not comprise a sequence that is complementary to a sequence of the amplification product. Non-limiting examples of tags are described in the foregoing and following disclosure.

In some instances, detecting an amplification product, whether tagged or not tagged, comprises subjecting the amplification product to a signal detector or assay assembly of a device, system, or kit disclosed herein. In some instances, methods comprise comprises amplifying and detecting on an assay assembly of a device, system, or kit disclosed herein. In some instances, the assay assembly comprises amplification reagents.

In some instances, detecting a nucleic acid does not comprise amplifying the nucleic acid or portion thereof. In some instances, detecting a nucleic acid does not comprise sequencing the nucleic acid or portion thereof. In some instances, detecting a nucleic acid does not comprise sequencing or amplifying the nucleic acid or portion thereof. For example, in some instances, a nucleic acid may tagged with a labeled probe and detection of the labeled probe is sufficient to detect the absence, presence or quantity of the nucleic acid. Thus, devices and systems disclosed herein capable of performing such methods may not comprise an amplification reagent, a sequencing apparatus, or a combination thereof.

In some instances, detecting comprises subjecting a biomarker to a lateral flow assay. Detecting may further comprise applying an instrument or reagent to the lateral flow assay to control the flow of a biological sample, solution, or combination thereof, through the lateral flow assay. In some instances, the instrument is a vacuum, a pump, a pipet, or a combination thereof.

In some instances, methods comprise detecting a highly repetitive region (e.g., HRR). A highly repetitive region may be a region that comprises at least two sequences that are at least 50% identical. In some instances, the highly repetitive region comprises at least 3, at least 4, at least 5, at least 6, at least 7, at least 8, at least 9, or at least 10 sequences that are at least 50% identical. In some instances, the at least two regions are at least about 50%, at least about 60%, at least about 70%, at least about 80%, at least about 90%, at least about 95%, at least about 99%, or 100% identical. In some instances, the at least two sequences should be sufficiently far apart that they appear in two separate cell-free DNA fragments. In some instances, the at least two sequences are separated by at least one nucleotide. In some instances, the at least two sequences are separated by at least two nucleotides. In some instances, the at least two sequences are separated by at least about 5, at least about 10, at least about 15, at least about 20, at least about 30, at least about 40, at least about 50 nucleotides. In some instances, the at least two sequences are separated by up to about 200 nucleotides. By way of non-limiting example, the HRR may be a highly repetitive Y chromosome region (HRYR).

In some instances, methods comprise detecting a number of copies of a sequence of interest. In some instances, the number of copies is between 1 and about 50,000. In some instances, the number of copies is between about 1 and about 50. In some instances, the number of copies is between 1 and about 500. In some instances, the number of copies is between 1 and about 1,000. In some instances, the number of copies is between 1 and about 2,000. In some instances, the number of copies is between 1 and about 5,000. In some instances, the number of copies is less than about 10,000. In some instances, the number of copies is less than about 5,000. In some instances, the number of copies is between 4 and about 20,000. In some instances, the number of copies is between 4 and about 10,000. In some instances, the number of copies is between 4 and about 5,000. In some instances, the number of copies is between 4 and about 1,000. In some instances, the number of copies is less than about 1,000. In some instances, the number of copies is less than about 500. In some instances, the number of copies is less than about 200. In some instances, the number of copies is less than about 100. In some instances, the number of copies is less than about 50. In some instances, the number of copies is less than about 40. In some instances, the number of copies is less than about 20. In some instances, the number of copies is at least 1. In some instances, the number of copies is at least 2. In some instances, the number of copies is at least 4. In some instances, the number of copies is at least 5. In some instances, the number of copies is at least 10. In some instances, the sequence of interest is a sequence specific to a Y chromosome. By way of non-limiting example, methods may comprise detecting a male fetus as long as one copy of the Y chromosome region, or one fragment of the Y chromosome containing the sequence of interest, is present in a sample.

In some instances, methods disclosed herein comprise detecting a circulating cell free nucleic acid corresponding to a Y chromosome region. In some instances, the cell free nucleic acid comprises a sequence that is found on the Y chromosome. In some instances, the cell free nucleic acid comprises a sequence that is only found on the Y chromosome. In some instances, the cell free nucleic acid comprises a sequence that is not found on an X chromosome or any autosome. In some instances, the Y chromosome sequence is a sequence that occurs more than once on the Y chromosome. In some instances, the Y chromosome sequence is a first sequence that is a homolog of a second sequence, wherein the second sequence is also found on the Y chromosome. In some instances the first sequence is at least 80% identical to the second sequence. In some instances the first sequence is at least 85% identical to the second sequence. In some instances the first sequence is at least 90% identical to the second sequence. In some instances the first sequence is at least 95% identical to the second sequence. In some instances, the first sequence and the second sequence are at least 15 nucleotides in length. In some instances, the first sequence and the second sequence are at least 25 nucleotides in length. In some instances, the first sequence and the second sequence are at least 50 nucleotides in length. In some instances, the first sequence and the second sequence are at least 100 nucleotides in length.

In some instances, methods comprise detecting a nucleic acid corresponding to a Y chromosome region, or portion thereof, comprises a sequence that is present on the Y chromosome more than once. In some instances, the Y chromosome region is located between position 20000000 and position 21000000 of the Y chromosome. In some instances, the Y chromosome region is located between position 20500000 and position 21000000 of the Y chromosome. In some instances, the Y chromosome region is located between position 20000000 and position 20500000 of the Y chromosome. In some instances, the Y chromosome region is located between position 20000000 and position 20250000 of the Y chromosome. In some instances, the Y chromosome region is located between position 20250000 and position 20500000 of the Y chromosome. In some instances, the Y chromosome region is located between position 20500000 and position 20750000 of the Y chromosome. In some instances, the Y chromosome region is located between position 20750000 and position 21000000 of the Y chromosome. In some instances, the Y chromosome region is located between position 20080000 and position 20400000 of the Y chromosome. In some instances, the Y chromosome region is located between position 20082000 and position 20351000 of the Y chromosome. In some instances, the Y chromosome region is located between position 20082183 and position 20350897 of the Y chromosome. In some instances, corresponding is 100% identical. In some instances, corresponding is at least 99% identical. In some instances, corresponding is at least 98% identical. In some instances, corresponding is at least 95% identical. In some instances, corresponding is at least 90% identical.

In some instances, methods disclosed herein comprise detecting or quantifying circulating cell free nucleic acids comprising a sequence corresponding to a Y chromosome region, or portion thereof, located between position 20000000 and position 21000000 of the Y chromosome, wherein the Y chromosome region has a given length. In some instances, the Y chromosome region is about 10 nucleotides to about 1,000,000 nucleotides in length. In some instances, the Y chromosome region is about 10 nucleotides to about 500,000 nucleotides in length. In some instances, the Y chromosome region is about 10 nucleotides to about 300,000 nucleotides in length. In some instances, the Y chromosome region is about 100 nucleotides to about 1,000,000 nucleotides in length. In some instances, the Y chromosome region is about 100 nucleotides to about 500,000 nucleotides in length. In some instances, the Y chromosome region is about 100 nucleotides to about 300,000 base pairs in length. In some instances, the Y chromosome region is about 1000 nucleotides to about 1,000,000 nucleotides in length. In some instances, the Y chromosome region is about 1000 nucleotides to about 500,000 nucleotides in length. In some instances, the Y chromosome region is about 1000 nucleotides to about 300,000 nucleotides in length. In some instances, the Y chromosome region is about 10,000 nucleotides to about 1,000,000 nucleotides in length. In some instances, the Y chromosome region is about 10,000 nucleotides to about 500,000 nucleotides in length. In some instances, the Y chromosome region is about 10,000 nucleotides to about 300,000 nucleotides in length. In some instances, the Y chromosome region is about 300,000 nucleotides in length.

In some instances, methods disclosed herein comprise detecting or quantifying circulating cell free nucleic acids comprising a sequence corresponding to a Y chromosome region, or portion thereof, located between position 20000000 and position 21000000 of the Y chromosome, wherein the sequence has a given length. In some instances, methods disclosed herein comprise detecting circulating cell free nucleic acids comprising a sequence corresponding to a Y chromosome region. In some instances, the sequence is about 10 nucleotides to about 1,000 nucleotides in length. In some instances, the sequence is about 10 nucleotides to about 500 nucleotides in length. In some instances, the sequence is about 10 nucleotides to about 400 nucleotides in length. In some instances, the sequence is about 10 nucleotides to about 300 nucleotides in length. In some instances, the sequence is about 50 nucleotides to about 1000 nucleotides in length. In some instances, the sequence is about 50 nucleotides to about 500 nucleotides in length.

In some instances, methods disclosed herein comprise detecting or quantifying circulating cell free nucleic acids comprising a sequence corresponding to a Y chromosome region, or portion thereof, wherein the portion thereof has a given length. In some instances, the length of the portion thereof is about 10 nucleotides to about 100 nucleotides. In some instances, the length of the portion thereof is about 100 nucleotides to about 1000 nucleotides. In some instances, the length of the portion thereof is about 1000 nucleotides to about 10,000 nucleotides. In some instances, the length of the portion thereof is about 10,000 nucleotides to about 100,000 nucleotides.

In some instances, methods disclosed herein comprise detecting at least one circulating cell free nucleic acid comprising a sequence corresponding to a Y chromosome sub-region of a Y chromosome region disclosed herein. In some instances, the sub-region is represented by a sequence that is present in the Y chromosome region more than once. In some instances, corresponding is 100% identical. In some instances, corresponding is at least 99% identical. In some instances, corresponding is at least 98% identical. In some instances, corresponding is at least 95% identical. In some instances, corresponding is at least 90% identical.

In some instances, methods disclosed herein comprise detecting a circulating cell free nucleic acid comprising a sequence corresponding to a Y chromosome sub-region between start position 20350799 and end position 20350897 of the Y chromosome. In some instances, methods disclosed herein comprise detecting a circulating cell free nucleic acid comprising a sequence corresponding to at least 10 nucleotides of a Y chromosome sub-region between start position 20350799 and end position 20350897 of the Y chromosome. In some instances, methods disclosed herein comprise detecting a circulating cell free nucleic acid comprising a sequence corresponding to at least 100 nucleotides of a Y chromosome sub-region between start position 20350799 and end position 20350897 of the Y chromosome. In some instances, methods disclosed herein comprise detecting a circulating cell free nucleic acid comprising a sequence corresponding to at least 200 nucleotides of a Y chromosome sub-region between start position 20350799 and end position 20350897 of the Y chromosome. In some instances, methods disclosed herein comprise detecting a circulating cell free nucleic acid comprising a sequence corresponding to at least 500 nucleotides of a Y chromosome sub-region between start position 20350799 and end position 20350897 of the Y chromosome.

In some instances, methods disclosed herein comprise detecting a circulating cell free nucleic acid comprising a sequence corresponding to a Y chromosome sub-region between start position 56673250 and end position 56771489 of the Y chromosome. In some instances, the sequence corresponds to at least 10 nucleotides of a Y chromosome sub-region between start position 56673250 and end position 56771489 of the Y chromosome. In some instances, the sequence corresponds to at least 50 nucleotides of a Y chromosome sub-region between start position 56673250 and end position 56771489 of the Y chromosome. In some instances, the sequence corresponds to at least about 10 to at least about 1000 nucleotides of a Y chromosome sub-region between start position 56673250 and end position 56771489 of the Y chromosome. In some instances, the sequence corresponds to at least about 50 to at least about 500 nucleotides of a Y chromosome sub-region between start position 56673250 and end position 56771489 of the Y chromosome. In some instances, the sequence corresponds to at least about 50 to at least about 150 nucleotides of a Y chromosome sub-region between start position 56673250 and end position 56771489 of the Y chromosome.

In some instances, methods disclosed herein comprise detecting a circulating cell free nucleic acid comprising a sequence corresponding to a Y chromosome sub-region between start position 20349236 and end position 20349318 of the Y chromosome. In some instances, methods disclosed herein comprise detecting a circulating cell free nucleic acid comprising a sequence corresponding to at least 10 nucleotides of a Y chromosome sub-region between start position 20349236 and end position 20349318 of the Y chromosome. In some instances, methods disclosed herein comprise detecting a circulating cell free nucleic acid comprising a sequence corresponding to at least 100 nucleotides of a Y chromosome sub-region between start position 20349236 and end position 20349318 of the Y chromosome. In some instances, methods disclosed herein comprise detecting a circulating cell free nucleic acid comprising a sequence corresponding to at least 200 nucleotides of a Y chromosome sub-region between start position 20349236 and end position 20349318 of the Y chromosome. In some instances, methods disclosed herein comprise detecting a circulating cell free nucleic acid comprising a sequence corresponding to at least 500 nucleotides of a Y chromosome sub-region between start position 20349236 and end position 20349318 of the Y chromosome.

In some instances, methods disclosed herein comprise detecting a circulating cell free nucleic acid comprising a sequence corresponding to a Y chromosome sub-region between start position 20350231 and end position 20350323 of the Y chromosome. In some instances, methods disclosed herein comprise detecting a circulating cell free nucleic acid comprising a sequence corresponding to at least 10 nucleotides of a Y chromosome sub-region between start position 20350231 and end position 20350323 of the Y chromosome. In some instances, methods disclosed herein comprise detecting a circulating cell free nucleic acid comprising a sequence corresponding to at least 100 nucleotides of a Y chromosome sub-region between start position 20350231 and end position 20350323 of the Y chromosome. In some instances, methods disclosed herein comprise detecting a circulating cell free nucleic acid comprising a sequence corresponding to at least 200 nucleotides of a Y chromosome sub-region between start position 20350231 and end position 20350323 of the Y chromosome. In some instances, methods disclosed herein comprise detecting a circulating cell free nucleic acid comprising a sequence corresponding to at least 500 nucleotides of a Y chromosome sub-region between start position 20350231 and end position 20350323 of the Y chromosome.

In some instances, methods disclosed herein comprise detecting a circulating cell free nucleic acid comprising a sequence corresponding to a Y chromosome sub-region between start position 20350601 and end position 20350699 of the Y chromosome. In some instances, methods disclosed herein comprise detecting a circulating cell free nucleic acid comprising a sequence corresponding to at least 10 nucleotides of a Y chromosome sub-region between start position 20350601 and end position 20350699 of the Y chromosome. In some instances, methods disclosed herein comprise detecting a circulating cell free nucleic acid comprising a sequence corresponding to at least 100 nucleotides of a Y chromosome sub-region between start position 20350601 and end position 20350699 of the Y chromosome. In some instances, methods disclosed herein comprise detecting a circulating cell free nucleic acid comprising a sequence corresponding to at least 200 nucleotides of a Y chromosome sub-region between start position 20350601 and end position 20350699 of the Y chromosome. In some instances, methods disclosed herein comprise detecting a circulating cell free nucleic acid comprising a sequence corresponding to at least 500 nucleotides of a Y chromosome sub-region between start position 20350601 and end position 20350699 of the Y chromosome.

In some instances, methods disclosed herein comprise detecting a circulating cell free nucleic acid comprising a sequence corresponding to a Y chromosome sub-region between start position 20082183 and end position 20082281 of the Y chromosome. In some instances, methods disclosed herein comprise detecting a circulating cell free nucleic acid comprising a sequence corresponding to at least 10 nucleotides of a Y chromosome sub-region between start position 20082183 and end position 20082281 of the Y chromosome. In some instances, methods disclosed herein comprise detecting a circulating cell free nucleic acid comprising a sequence corresponding to at least 100 nucleotides of a Y chromosome sub-region between start position 20082183 and end position 20082281 of the Y chromosome. In some instances, methods disclosed herein comprise detecting a circulating cell free nucleic acid comprising a sequence corresponding to at least 200 nucleotides of a Y chromosome sub-region between start position 20082183 and end position 20082281 of the Y chromosome. In some instances, methods disclosed herein comprise detecting a circulating cell free nucleic acid comprising a sequence corresponding to at least 500 nucleotides of a Y chromosome sub-region between start position 20082183 and end position 20082281 of the Y chromosome.

In some instances, methods disclosed herein comprise detecting circulating cell free nucleic acids comprising a sequence corresponding to a Y chromosome sub-region, wherein the sequence is selected from SEQ ID NOS.: 1-5, 30-34, and 141-192. In some instances, the sequence is at least 60% identical to a sequence selected from SEQ ID NOS.: 1-5, 30-34, and 141-192. In some instances, the sequence is at least 65% identical to a sequence selected from SEQ ID NOS.: 1-5, 30-34, and 141-192. In some instances, the sequence is at least 70% identical to a sequence selected from SEQ ID NOS.: 1-5, 30-34, and 141-192. In some instances, the sequence is at least 75% identical to a sequence selected from SEQ ID NOS.: 1-5, 30-34, and 141-192. In some instances, the sequence is at least 80% identical to a sequence selected from SEQ ID NOS.: 1-5, 30-34, and 141-192. In some instances, the sequence is at least 85% identical to a sequence selected from SEQ ID NOS.: 1-5, 30-34, and 141-192. In some instances, the sequence is at least 90% identical to a sequence selected from SEQ ID NOS.: 1-5, 30-34, and 141-192. In some instances, the sequence is at least 95% identical to a sequence selected from SEQ ID NOS.: 1-5, 30-34, and 141-192. In some instances, the sequence is at least 98% identical to a sequence selected from SEQ ID NOS.: 1-5, 30-34, and 141-192. In some instances, the sequence is at least 99% identical to a sequence selected from SEQ ID NOS.: 1-5, 30-34, and 141-192. In some instances, the sequence is 100% identical to a sequence selected from SEQ ID NOS.: 1-5, 30-34, and 141-192.

In some instances, methods comprise detecting a nucleic acid corresponding to Y chromosome sequence having a homolog or copy on the Y chromosome is a sequence present in a Y chromosome gene. In some instances, the Y chromosome sequence is located in a repeat region of the Y chromosome. In some instances a repeat region comprises a pseudogene, a near exact copy of a gene (>90% homologous when aligned for maximal homology), intergenic region, or microsatellite repeat, or a recognizable portion thereof (e.g., at least 10 nucleotides). Non-limiting examples of Y chromosome genes are testis specific protein Y-Linked 1 (TSPY1), (alias DYS14), testis specific protein Y-Linked 2 (TSPY2), DYZ1, testis-specific transcript Y linked 22 (TTTY22), sex determining region Y (SRY), ribosomal protein S4 Y-linked 1 (RPS4Y1), zinc finger protein Y-linked (ZFY), TGIF2LY. In some instances, the Y chromosome sequence comprises a sequence selected from SEQ ID NOS.: 1-5, 30-34, and 141-192. In some instances, the Y chromosome sequence comprises a sequence that is at least 90% identical to a sequence selected from SEQ ID NOS.: 1-5, 30-34, and 141-192. In some instances, the Y chromosome sequence comprises at least 10 consecutive nucleotides that are identical to at least 10 consecutive nucleotides of a sequence selected from SEQ ID NOS.: 1-5, 30-34, and 141-192. In some instances, the Y chromosome sequence comprises at least 20 consecutive nucleotides that are identical to at least 20 consecutive nucleotides of a sequence selected from SEQ ID NOS.: 1-5, 30-34, and 141-192. In some instances, the Y chromosome sequence comprises at least 50 consecutive nucleotides that are identical to at least 50 consecutive nucleotides of a sequence selected from SEQ ID NOS.: 1-5, 30-34, and 141-192. In some instances, the Y chromosome sequence comprises at least 100 consecutive nucleotides that are identical to at least 100 consecutive nucleotides of a sequence selected from SEQ ID NOS.: 1-5, 30-34, and 141-192.

Detecting may comprise viewing an interface of a device or system disclosed herein where the result of a test is displayed. Detecting may comprise viewing a color appearance or fluorescent signal on a lateral flow device. Detecting may comprise receiving a result of a test on a device disclosed herein. Detecting may comprise receiving a result of a test on a mobile device, computer, notepad or other electronic device in communication with a device of system disclosed herein.

Generally, the methods, kits, systems and devices disclosed herein are capable of providing genetic information (e.g., fetus gender) in a short amount of time. In some instances, methods disclosed herein can be performed in less than about 1 minute. In some instances, methods disclosed herein can be performed in less than about 2 minutes. In some instances, methods disclosed herein can be performed in less than about 5 minutes. In some instances, methods disclosed herein can be performed in less than about 10 minutes. In some instances, methods disclosed herein can be performed in less than about 15 minutes. In some instances, methods disclosed herein can be performed in less than about 20 minutes. In some instances, methods disclosed herein can be performed in less than about 30 minutes. In some instances, methods disclosed herein can be performed in less than about 45 minutes. In some instances, methods disclosed herein can be performed in less than about 60 minutes. In some instances, methods disclosed herein can be performed in less than about 90 minutes. In some instances, methods disclosed herein can be performed in less than about 2 hours. In some instances, methods disclosed herein can be performed in less than about 3 hours. In some instances, methods disclosed herein can be performed in less than about 4 hours.

Use of methods, kits, systems and devices disclosed herein generally does not require any technical training. For instance, kits, systems and devices disclosed herein may be used by the pregnant subject in her home without the assistance of a technician or medical provider. In some instances, methods disclosed herein can be performed by a user with no medical training or technical training. In some instances, methods, kits, systems and devices disclosed herein simply require that a user add a biological sample to the system or device, optionally power on the system or device, and view a result to obtain genetic information.

### III. Aspects Related to Devices, Systems, Kits and Methods

The following aspects are related to devices, systems, kits and methods disclosed herein. Devices, systems, kits and methods disclosed herein are generally designed to process and analyze biomarkers and nucleic acids in biological samples of animal subjects, plants, and environmental samples. The following descriptions of biological samples, cell-free nucleic acids, and subjects may aid in understanding the utility of devices, systems, kits and methods disclosed herein.

### Biological Samples

Disclosed herein are devices, systems, kits and methods for analyzing biomarkers and nucleic acids in a biological sample. In general, biological samples include animal samples, plant samples, and environmental samples. Non-limiting examples of animal samples are blood and urine. Non-limiting examples of plant samples are leafy matter and seeds. Non-limiting examples of environmental samples are water samples a body of water (e.g., ocean, lake, river, stream), treated water, industrial waste, soil samples, food samples. In some instances, the biological sample must be prepared in the form of a fluid solution before it can be employed by a device, system, kit or method disclosed herein.

In some instances, the biological sample is a biological fluid sample. Non-limiting examples of biological fluid samples include samples of whole blood, plasma, serum, saliva, urine, sweat, tears, rectal discharge, cerebrospinal fluid, lymphatic fluid, synovial fluid, interstitial fluid, and vaginal fluid. In some instances, the biological sample comprises whole blood. Whole blood, in contrast to plasma, requires little processing. There may be a filtration step to remove some debris from the blood sample without separating red blood cells from white blood cells. In some instances, the biological sample is a swab, e.g., a buccal swab or vaginal swab.

Biological samples described herein include biological fluids that are substantially acellular or can be modified to be acellular biological fluids. For instance, the cell-free nucleic acid may be circulating in the bloodstream of the subject, and therefore the detection reagent may be used to detect or quantify the marker in a blood or serum sample from the subject. The terms “plasma” and “serum” are used interchangeably herein, unless otherwise noted. However, in some cases they are included in a single list of sample species to indicate that both are covered by the description or claim.

In some instances, devices, systems, kits and methods disclosed herein are capable of removing cells from a biological sample. The resulting sample may be referred to as a cell-depleted sample. The cell-depleted sample may have at least 95% fewer whole, intact cells than the biological sample. The cell-depleted sample may have at least 90% fewer whole, intact cells than the biological sample. The cell-depleted sample may have at least 80% fewer whole, intact cells than the biological sample. The cell-depleted sample may have at least about 75%, at least about 70%, at least about 60%, at least about 50%, at least about 40%, or at least about 25% fewer whole, intact cells than the biological sample. The cell-depleted sample may be completely free of any whole, intact cells.

In some instances, the biological sample comprises capillary blood. In some instances, the biological sample comprises venous blood. Blood obtained from capillaries (e.g., blood vessels of extremities like fingers, toes) may be referred to herein as “capillary blood.” Blood obtained from veins (e.g., arm, middle of hand) may be referred to herein as “venous blood.” Common veins for venipuncture to obtain venous blood are the median cubital vein, cephalic vein, basilic vein, and dorsal metacarpal veins. In some instances, the biological sample consists essentially of capillary blood. In some instances, the biological sample consists of capillary blood. In some embodiments, the biological sample does not comprise venous blood. In some instances, the biological sample comprises plasma. In some instances, the biological sample consists essentially of plasma. In some instances, the biological sample consists of plasma. In some instances, the biological sample comprises serum. In some instances, the biological sample consists essentially of serum. In some instances, the biological sample consists of serum. In some instances, the biological sample comprises urine. In some instances, the biological sample consists essentially of urine. In some instances, the biological sample consists of urine. In some instances, the biological sample comprises saliva. In some instances, the biological sample consists essentially of saliva. In some instances, the biological sample consists of saliva. In some instances, the biological fluid comprises vaginal fluid. In some instances, the biological fluid consists essentially of vaginal fluid. In some instances, the biological fluid consists of vaginal fluid. In some instances, the vaginal fluid is obtained by performing a vaginal swab of the pregnant subject. In some instances, the biological sample comprises interstitial fluid. In some instances, the biological sample consists essentially of interstitial fluid. In some instances, the biological sample consists of interstitial fluid.

In some instances, the biological sample is whole blood. Generally, the devices, systems, kits, and methods disclosed herein are capable of analyzing cell free nucleic acids from very small samples of whole blood. In some instances, the small sample of whole blood maybe obtained with a finger prick, such as performed with a lancet or pin/needle. In some instances, the small sample of whole blood maybe obtained without a phlebotomy. In some instances, devices, systems, kits, and methods disclosed herein are capable of analyzing cell free nucleic acids in whole blood without the separation of whole blood into blood fractions (serum, plasma, cellular fraction).

In some instances, the devices, systems, kits, and methods disclosed herein require at least about 20 μL of blood to provide a test result with at least about 95% confidence or accuracy. In some instances, the devices, systems and kits disclosed herein require at least about 30 μL of blood to provide a test result with at least about 95% confidence or accuracy. In some instances, the devices, systems and kits disclosed herein require at least about 40 μL of blood to provide a test result with at least about 95% confidence or accuracy. In some instances, the devices, systems and kits disclosed herein require at least about 50 μL of blood to provide a test result with at least about 95% confidence or accuracy. In some instances, the devices, systems and kits disclosed herein require at least about 60 μL of blood to provide a test result with at least about 95% confidence or accuracy. In some instances, the devices, systems and kits disclosed herein require at least about 70 μL of blood to provide a test result with at least about 95% confidence or accuracy. In some instances, the devices, systems and kits disclosed herein require at least about 20 μL of blood to provide a test result with at least about 99% confidence or accuracy. In some instances, the devices, systems and kits disclosed herein require at least about 20 μL of blood to provide a test result with at least about 99% confidence or accuracy. In some instances, the devices, systems and kits disclosed herein require at least about 40 μL of blood to provide a test result with at least about 99% confidence or accuracy. In some instances, the devices, systems and kits disclosed herein require at least about 60 μL of blood to provide a test result with at least about 99% confidence or accuracy. In some instances, the devices, systems and kits disclosed herein require at least about 80 μL of blood to provide a test result with at least about 99% confidence or accuracy. In some instances, the devices, systems and kits disclosed herein require at least about 100 μL of blood to provide a test result with at least about 90% confidence or accuracy. In some instances, the method comprise obtaining only about 20 μL to about 100 μL of blood to provide a test result with at least about 95% confidence or accuracy. In some instances, the devices, systems and kits disclosed herein require only about 20 μL to about 100 μL of blood to provide a test result with at least about 98% confidence or accuracy. In some instances, the devices, systems and kits disclosed herein require only about 20 μL to about 100 μL of blood to provide a test result with at least about 99% confidence or accuracy. In some instances, the devices, systems and kits disclosed herein require only about 20 μL to about 100 μL of blood to provide a test result with about 99.5% confidence or accuracy. In some instances, the devices, systems and kits disclosed herein require only about 20 μL to about 100 μL of blood to provide a test result with about 99.9% confidence or accuracy.

In some instances, the biological sample is plasma. Plasma makes up roughly 55% of whole blood. In some instances, the devices, systems, kits, and methods disclosed herein require at least about 10 μL of plasma to provide a test result with at least about 95% confidence or accuracy. In some instances, the devices, systems and kits disclosed herein require at least about 20 μL of plasma to provide a test result with at least about 95% confidence or accuracy. In some instances, the devices, systems and kits disclosed herein require at least about 30 μL of plasma to provide a test result with at least about 95% confidence or accuracy. In some instances, the devices, systems and kits disclosed herein require at least about 40 μL of plasma to provide a test result with at least about 95% confidence or accuracy. In some instances, the devices, systems and kits disclosed herein require at least about 50 μL of plasma to provide a test result with at least about 95% confidence or accuracy. In some instances, the devices, systems and kits disclosed herein require at least about 10 μL of plasma to provide a test result with at least about 99% confidence or accuracy. In some instances, the devices, systems and kits disclosed herein require at least about 20 μL of plasma to provide a test result with at least about 99% confidence or accuracy. In some instances, the devices, systems and kits disclosed herein require at least about 30 μL of plasma to provide a test result with at least about 99% confidence or accuracy. In some instances, the devices, systems and kits disclosed herein require at least about 40 μL of plasma to provide a test result with at least about 99% confidence or accuracy. In some instances, the devices, systems and kits disclosed herein require at least about 50 μL of plasma to provide a test result with at least about 99% confidence or accuracy. In some instances, the devices, systems and kits disclosed herein require only about 10 μL to about 50 μL of plasma to provide a test result with at least about 95% confidence or accuracy. In some instances, the devices, systems and kits disclosed herein require only about 20 μL to about 60 μL of plasma to provide a test result with at least about 95% confidence or accuracy. In some instances, the devices, systems and kits disclosed herein require only about 10 μL to about 50 μL of plasma to provide a test result with at least about 99% confidence or accuracy.

In some instances, the biological sample is saliva. In some instances, the devices, systems, kits, and methods disclosed herein require at least about 100 μL of saliva to provide a test result with at least about 95% confidence or accuracy. In some instances, the devices, systems, kits, and methods disclosed herein require at least about 200 μL of saliva to provide a test result with at least about 95% confidence or accuracy. In some instances, the devices, systems, kits, and methods disclosed herein require at least about 500 μL of saliva to provide a test result with at least about 95% confidence or accuracy. In some instances, the devices, systems, kits, and methods disclosed herein require at least about 1 ml of saliva to provide a test result with at least about 95% confidence or accuracy. In some instances, the devices, systems, kits, and methods disclosed herein require at least about 2 ml of saliva to provide a test result with at least about 95% confidence or accuracy. In some instances, the devices, systems, kits, and methods disclosed herein require at least about 3 ml of saliva to provide a test result with at least about 95% confidence or accuracy.

In some instances, the biological sample is vaginal fluid. In some instances, the devices, systems, kits, and methods disclosed herein require at least about 50 μL of vaginal fluid to provide a test result with at least about 95% confidence or accuracy. In some instances, the devices, systems, kits, and methods disclosed herein require at least about 100 μL of vaginal fluid to provide a test result with at least about 95% confidence or accuracy. In some instances, the devices, systems, kits, and methods disclosed herein require at least about 200 μL of vaginal fluid to provide a test result with at least about 95% confidence or accuracy. In some instances, the devices, systems, kits, and methods disclosed herein require at least about 500 μL of vaginal fluid to provide a test result with at least about 95% confidence or accuracy. In some instances, the devices, systems, kits, and methods disclosed herein require at least about 1 ml of vaginal fluid to provide a test result with at least about 95% confidence or accuracy. In some instances, the devices, systems, kits, and methods disclosed herein require at least about 2 ml of vaginal fluid to provide a test result with at least about 95% confidence or accuracy. In some instances, the devices, systems, kits, and methods disclosed herein require at least about 3 ml of vaginal fluid to provide a test result with at least about 95% confidence or accuracy.

### Cell Free Nucleic Acids

In some instances, the methods, devices, systems and kits disclosed herein are useful for evaluating a cell-free nucleic acid in a biological sample. In some instances, the cell-free nucleic acid is DNA (cf-DNA), or RNA (cf-RNA). In some instances, the cell-free nucleic acid is a fetal nucleic acid. In some instances, the cell-free fetal nucleic acid is a cell-free fetal DNA (cff-DNA) or cell-free fetal RNA (cff-RNA). In some instances, the cf-DNA or cff-DNA is a genomic DNA or a cDNA. In some instances, the cf-DNA comprises mitochondrial DNA. In some instances, the cf-RNA or cff-RNA is a messenger RNA (mRNA), a microRNA (miRNA), mitochondrial RNA, or a natural antisense RNA (NAS-RNA). In some instances, the cell-free nucleic acid is a mixture of maternal and fetal nucleic acid. A cell-free fetal nucleic acid that circulates in the maternal bloodstream can be referred to as a “circulating cell-free nucleic acid” or a “circulatory extracellular DNA.” In some instances, the cell-free nucleic acid comprises epigenetic modifications. In some instances, the cell-free nucleic acid comprises a pattern of epigenetic modifications that corresponds to gender or other genetic information of interest. In some instances, the cell-free nucleic acid comprises methylated cytosines. In some instances, the cell-free nucleic acid comprises a cytosine methylation pattern that corresponds to gender or other genetic information of interest.

In some instances, methods, devices, systems and kits disclosed herein are configured to detect or quantify cellular nucleic acids, such as nucleic acids from disrupted cells or lysed cells. In some instances, cellular nucleic acids are from cells that are intentionally disrupted or lysed. In some instances, cellular nucleic acids are from cells that are unintentionally disrupted or lysed. Methods, devices, systems and kits disclosed herein may be configured to analyze intentionally disrupted or lysed cells, but not unintentionally disrupted or lysed cells. In some instances, less than about 0.1% of the total nucleic acids in the biological sample are cellular nucleic acids. In some instances, less than about 1% of the total nucleic acids in the biological sample are cellular nucleic acids. In some instances, less than about 5% of the total nucleic acids in the biological sample are cellular nucleic acids. In some instances, less than about 10% of the total nucleic acids in the biological sample are cellular nucleic acids. In some instances, less than about 20% of the total nucleic acids in the biological sample are cellular nucleic acids. In some instances, less than about 30% of the total nucleic acids in the biological sample are cellular nucleic acids. In some instances, less than about 40% of the total nucleic acids in the biological sample are cellular nucleic acids. In some instances, less than about 50% of the total nucleic acids in the biological sample are cellular nucleic acids. In some instances, less than about 60% of the total nucleic acids in the biological sample are cellular nucleic acids. In some instances, less than about 70% of the total nucleic acids in the biological sample are cellular nucleic acids. In some instances, less than about 80% of the total nucleic acids in the biological sample are cellular nucleic acids. In some instances, less than about 90% of the total nucleic acids in the biological sample are cellular nucleic acids.

### Experimental Controls

In some instances, devices, systems, kits and methods comprise an experimental control or use thereof. In some instances, the experimental control comprises a nucleic acid, a protein, a peptide, an antibody, an antigen binding antibody fragment, a binding moiety. In some instances, the experimental control comprises a signal for detecting the experimental control. Non-limiting examples of signals are fluorescent molecules, dye molecules, nanoparticles, and colorimetric indicators. In some instances, the experimental control comprises a cell free nucleic acid. In some instances, the cell free nucleic acid comprises a cell free fetal nucleic acid. In some instances, the cell free nucleic acid comprises a maternal cell free nucleic acid. In some instances, the cell free nucleic acid comprises a maternal cell free nucleic acid (e.g., to assess the amount of cellular disruption/lysis that occurs during sample processing). In some instances, the cell free nucleic acid comprises a sequence corresponding to a Y chromosome. In some instances, the cell free nucleic acid comprises a sequence corresponding to an X chromosome. In some instances, the cell free nucleic acid comprises a sequence corresponding to an autosome. In some instances, the experimental control is a fetal nucleic acid control. In some instances, there are differentially methylated regions of DNA that indicate a presence of fetal DNA. In some instances, the fetal DNA control provides confirmation of pregnancy. By way of non-limiting example, RASSF1A gene is reportedly hyper-methylated in placental cells and hypo-methylated in maternal blood cells.

In some instances, the biological sample is a maternal body fluid sample obtained from a pregnant subject, a subject suspected of being pregnant, or a subject that has given birth recently, e.g., within the past day. In some instances, the maternal body fluid sample comprises blood, e.g., whole blood, a peripheral blood sample, or a blood fraction (plasma, serum). In some instances, the maternal body fluid sample comprises sweat, tears, sputum, urine, ear flow, lymph, saliva, cerebrospinal fluid, bone marrow suspension, vaginal fluid, transcervical lavage, brain fluid, ascites, milk, secretions of the respiratory, intestinal and genitourinary tracts, amniotic fluid, or a leukophoresis sample. In some instances, the biological sample is a maternal body fluid sample that is can be obtained easily by non-invasive procedures, e.g., blood, plasma, serum, sweat, tears, sputum, urine, ear flow, or saliva. In some instances, the sample is a combination of at least two body fluid samples. In some instances, the cell-free fetal nucleic acid originates from the maternal placenta, e.g., from apoptosed placental cells. In some instances, the biological sample is placental blood.

In some instances, a nucleic acid evaluated or analyzed by devices, systems, kits, and methods disclosed herein has a preferable length. In some instances, the nucleic acid is a cell-free fetal DNA fragment. In some instances, the cell-free fetal DNA fragment is from a Y chromosome. In some instances, the nucleic acid is about 15 bp to about 500 bp in length. In some instances, the nucleic acid is about 50 bp in length to about 200 bp in length. In some instances, the nucleic acid is at least about 15 bp in length. In some instances, the nucleic acid is at most about 500 bp in length. In instances, the nucleic acid is about 15 bp in length to about 50 bp in length, about 15 bp in length to about 75 bp in length, about 15 bp in length to about 100 bp in length, about 15 bp in length to about 150 bp in length, about 15 bp in length to about 200 bp in length, about 15 bp in length to about 250 bp in length, about 15 bp in length to about 300 bp in length, about 15 bp in length to about 350 bp in length, about 15 bp in length to about 400 bp in length, about 15 bp in length to about 450 bp in length, about 15 bp in length to about 500 bp in length, about 50 bp in length to about 75 bp in length, about 50 bp in length to about 100 bp in length, about 50 bp in length to about 150 bp in length, about 50 bp in length to about 200 bp in length, about 50 bp in length to about 250 bp in length, about 50 bp in length to about 300 bp in length, about 50 bp in length to about 350 bp in length, about 50 bp in length to about 400 bp in length, about 50 bp in length to about 450 bp in length, about 50 bp in length to about 500 bp in length, about 75 bp in length to about 100 bp in length, about 75 bp in length to about 150 bp in length, about 75 bp in length to about 200 bp in length, about 75 bp in length to about 250 bp in length, about 75 bp in length to about 300 bp in length, about 75 bp in length to about 350 bp in length, about 75 bp in length to about 400 bp in length, about 75 bp in length to about 450 bp in length, about 75 bp in length to about 500 bp in length, about 100 bp in length to about 150 bp in length, about 100 bp in length to about 200 bp in length, about 100 bp in length to about 250 bp in length, about 100 bp in length to about 300 bp in length, about 100 bp in length to about 350 bp in length, about 100 bp in length to about 400 bp in length, about 100 bp in length to about 450 bp in length, about 100 bp in length to about 500 bp in length, about 150 bp in length to about 200 bp in length, about 150 bp in length to about 250 bp in length, about 150 bp in length to about 300 bp in length, about 150 bp in length to about 350 bp in length, about 150 bp in length to about 400 bp in length, about 150 bp in length to about 450 bp in length, about 150 bp in length to about 500 bp in length, about 200 bp in length to about 250 bp in length, about 200 bp in length to about 300 bp in length, about 200 bp in length to about 350 bp in length, about 200 bp in length to about 400 bp in length, about 200 bp in length to about 450 bp in length, about 200 bp in length to about 500 bp in length, about 250 bp in length to about 300 bp in length, about 250 bp in length to about 350 bp in length, about 250 bp in length to about 400 bp in length, about 250 bp in length to about 450 bp in length, about 250 bp in length to about 500 bp in length, about 300 bp in length to about 350 bp in length, about 300 bp in length to about 400 bp in length, about 300 bp in length to about 450 bp in length, about 300 bp in length to about 500 bp in length, about 350 bp in length to about 400 bp in length, about 350 bp in length to about 450 bp in length, about 350 bp in length to about 500 bp in length, about 400 bp in length to about 450 bp in length, about 400 bp in length to about 500 bp in length, or about 450 bp in length to about 500 bp in length. In some instances, the nucleic acid is about 15 bp in length, about 50 bp in length, about 75 bp in length, about 100 bp in length, about 150 bp in length, about 200 bp in length, about 250 bp in length, about 300 bp in length, about 350 bp in length, about 400 bp in length, about 450 bp in length, or about 500 bp in length.

The sizes of the cell-free nucleic acids evaluated using the methods, devices, systems and kits disclosed herein can vary depending upon, e.g., the particular body fluid sample used. For example, cff-DNA sequences have been observed to be shorter than maternal cf-DNA sequences, and both cff-DNA and maternal cf-DNA to be shorter in urine than in plasma samples.

In some instances, the cff-DNA sequences evaluated in urine range from about 20 bp to about 300 bp in length. In some instances, the cff-DNA sequences evaluated in a urine sample are about 15 bp in length to about 300 bp in length. In some instances, the cff-DNA sequences evaluated in a urine sample are at least about 15 bp in length. In some instances, the cff-DNA sequences evaluated in a urine sample are at most about 300 bp in length. In some instances, the cff-DNA sequences evaluated in a urine sample are about 15 bp in length to about 20 bp in length, about 15 bp in length to about 30 bp in length, about 15 bp in length to about 60 bp in length, about 15 bp in length to about 90 bp in length, about 15 bp in length to about 120 bp in length, about 15 bp in length to about 150 bp in length, about 15 bp in length to about 180 bp in length, about 15 bp in length to about 210 bp in length, about 15 bp in length to about 240 bp in length, about 15 bp in length to about 270 bp in length, about 15 bp in length to about 300 bp in length, about 20 bp in length to about 30 bp in length, about 20 bp in length to about 60 bp in length, about 20 bp in length to about 90 bp in length, about 20 bp in length to about 120 bp in length, about 20 bp in length to about 150 bp in length, about 20 bp in length to about 180 bp in length, about 20 bp in length to about 210 bp in length, about 20 bp in length to about 240 bp in length, about 20 bp in length to about 270 bp in length, about 20 bp in length to about 300 bp in length, about 30 bp in length to about 60 bp in length, about 30 bp in length to about 90 bp in length, about 30 bp in length to about 120 bp in length, about 30 bp in length to about 150 bp in length, about 30 bp in length to about 180 bp in length, about 30 bp in length to about 210 bp in length, about 30 bp in length to about 240 bp in length, about 30 bp in length to about 270 bp in length, about 30 bp in length to about 300 bp in length, about 60 bp in length to about 90 bp in length, about 60 bp in length to about 120 bp in length, about 60 bp in length to about 150 bp in length, about 60 bp in length to about 180 bp in length, about 60 bp in length to about 210 bp in length, about 60 bp in length to about 240 bp in length, about 60 bp in length to about 270 bp in length, about 60 bp in length to about 300 bp in length, about 90 bp in length to about 120 bp in length, about 90 bp in length to about 150 bp in length, about 90 bp in length to about 180 bp in length, about 90 bp in length to about 210 bp in length, about 90 bp in length to about 240 bp in length, about 90 bp in length to about 270 bp in length, about 90 bp in length to about 300 bp in length, about 120 bp in length to about 150 bp in length, about 120 bp in length to about 180 bp in length, about 120 bp in length to about 210 bp in length, about 120 bp in length to about 240 bp in length, about 120 bp in length to about 270 bp in length, about 120 bp in length to about 300 bp in length, about 150 bp in length to about 180 bp in length, about 150 bp in length to about 210 bp in length, about 150 bp in length to about 240 bp in length, about 150 bp in length to about 270 bp in length, about 150 bp in length to about 300 bp in length, about 180 bp in length to about 210 bp in length, about 180 bp in length to about 240 bp in length, about 180 bp in length to about 270 bp in length, about 180 bp in length to about 300 bp in length, about 210 bp in length to about 240 bp in length, about 210 bp in length to about 270 bp in length, about 210 bp in length to about 300 bp in length, about 240 bp in length to about 270 bp in length, about 240 bp in length to about 300 bp in length, or about 270 bp in length to about 300 bp in length. In some instances, the cff-DNA sequences evaluated in a urine sample are about 15 bp in length, about 20 bp in length, about 30 bp in length, about 60 bp in length, about 90 bp in length, about 120 bp in length, about 150 bp in length, about 180 bp in length, about 210 bp in length, about 240 bp in length, about 270 bp in length, or about 300 bp in length.

In some instances, the cff-DNA sequences evaluated in a plasma or serum sample are at least about 20 bp in length. In some instances, the cff-DNA sequences evaluated in a plasma or serum sample are at least about 40 bp in length. In some instances, the cff-DNA sequences evaluated in a plasma or serum sample are at least about 80 bp in length. In some instances, the cff-DNA sequences evaluated in a plasma or serum sample are at most about 500 bp in length. In some instances, the cff-DNA sequences evaluated in plasma or serum range from about 100 bp to about 500 bp in length. In some instances, the cff-DNA sequences evaluated in a plasma or serum sample are about 50 bp in length to about 500 bp in length. In some instances, the cff-DNA sequences evaluated in a plasma or serum sample are about 80 bp in length to about 100 bp in length, about 80 bp in length to about 125 bp in length, about 80 bp in length to about 150 bp in length, about 80 bp in length to about 175 bp in length, about 80 bp in length to about 200 bp in length, about 80 bp in length to about 250 bp in length, about 80 bp in length to about 300 bp in length, about 80 bp in length to about 350 bp in length, about 80 bp in length to about 400 bp in length, about 80 bp in length to about 450 bp in length, about 80 bp in length to about 500 bp in length, about 100 bp in length to about 125 bp in length, about 100 bp in length to about 150 bp in length, about 100 bp in length to about 175 bp in length, about 100 bp in length to about 200 bp in length, about 100 bp in length to about 250 bp in length, about 100 bp in length to about 300 bp in length, about 100 bp in length to about 350 bp in length, about 100 bp in length to about 400 bp in length, about 100 bp in length to about 450 bp in length, about 100 bp in length to about 500 bp in length, about 125 bp in length to about 150 bp in length, about 125 bp in length to about 175 bp in length, about 125 bp in length to about 200 bp in length, about 125 bp in length to about 250 bp in length, about 125 bp in length to about 300 bp in length, about 125 bp in length to about 350 bp in length, about 125 bp in length to about 400 bp in length, about 125 bp in length to about 450 bp in length, about 125 bp in length to about 500 bp in length, about 150 bp in length to about 175 bp in length, about 150 bp in length to about 200 bp in length, about 150 bp in length to about 250 bp in length, about 150 bp in length to about 300 bp in length, about 150 bp in length to about 350 bp in length, about 150 bp in length to about 400 bp in length, about 150 bp in length to about 450 bp in length, about 150 bp in length to about 500 bp in length, about 175 bp in length to about 200 bp in length, about 175 bp in length to about 250 bp in length, about 175 bp in length to about 300 bp in length, about 175 bp in length to about 350 bp in length, about 175 bp in length to about 400 bp in length, about 175 bp in length to about 450 bp in length, about 175 bp in length to about 500 bp in length, about 200 bp in length to about 250 bp in length, about 200 bp in length to about 300 bp in length, about 200 bp in length to about 350 bp in length, about 200 bp in length to about 400 bp in length, about 200 bp in length to about 450 bp in length, about 200 bp in length to about 500 bp in length, about 250 bp in length to about 300 bp in length, about 250 bp in length to about 350 bp in length, about 250 bp in length to about 400 bp in length, about 250 bp in length to about 450 bp in length, about 250 bp in length to about 500 bp in length, about 300 bp in length to about 350 bp in length, about 300 bp in length to about 400 bp in length, about 300 bp in length to about 450 bp in length, about 300 bp in length to about 500 bp in length, about 350 bp in length to about 400 bp in length, about 350 bp in length to about 450 bp in length, about 350 bp in length to about 500 bp in length, about 400 bp in length to about 450 bp in length, about 400 bp in length to about 500 bp in length, or about 450 bp in length to about 500 bp in length. In some instances, the cff-DNA sequences evaluated in a plasma or serum sample are about 80 bp in length, about 100 bp in length, about 125 bp in length, about 150 bp in length, about 175 bp in length, about 200 bp in length, about 250 bp in length, about 300 bp in length, about 350 bp in length, about 400 bp in length, about 450 bp in length, or about 500 bp in length.

In some instances, the cell free nucleic acid comprises a sequence present in a human Y chromosome, referred to herein as “Y chromosome sequence,” unless otherwise specified. In some instances, the cell free nucleic acid comprises a sequence that is only found on the Y chromosome. In some instances, the cell free nucleic acid comprises a sequence that is not found on an X chromosome or any autosome. In some instances, at least a portion of the Y chromosome sequence is found in a Y chromosome protein-encoding gene. In some instances, at least a portion of the Y chromosome sequence is found in a Y chromosome non-encoding region. In some instances, at least a portion of the Y chromosome sequence is found in a Y chromosome protein-encoding gene exon. In some instances, at least a portion of the Y chromosome sequence is found in a Y chromosome protein-encoding gene intron. In some instances, at least a portion of the Y chromosome sequence has at least one homolog on the Y chromosome. In some instances, the Y chromosome sequence has at least two homologs on the Y chromosome. In some instances, the Y chromosome sequence is present in at least one copy on the Y chromosome. In some instances, the Y chromosome sequence is present in at least two copies on the Y chromosome. In some instances, the Y chromosome sequence is a sequence that is repeated at least once on the Y chromosome. In some instances, the Y chromosome sequence is a sequence that is repeated at least twice on the Y chromosome. In some instances, the Y chromosome sequence is not found on any other chromosome other than the Y chromosome. In some instances, the Y chromosome sequence is not found on an X chromosome. Non-limiting examples of regions or genes on the Y-chromosomes that have at least one homolog, copy or repeat on the Y chromosome are TSPY (alias DYS14), DYZ1, HSAY, TTTY22, SRY, RPS4Y1, ZFY, and TGIF2LY. Additional regions or genes on the Y-chromosomes that have at least one homolog, copy or repeat on the Y chromosome are disclosed herein.

### Subjects

Disclosed herein are devices, systems, kits and methods for analyzing a biological component in a sample from a subject. The subject may be human. The subject may be non-human. The subject may be non-mammalian (e.g., bird, reptile, insect). In some instances, the subject is a mammal. In some instances, the mammal is female. In some instances, the subject is a human subject. In some instances, the mammal is a primate (e.g., human, great ape, lesser ape, monkey). In some instances, the mammal is canine (e.g., dog, fox, wolf). In some instances, the mammal is feline (e.g., domestic cat, big cat). In some instances, the mammal is equine (e.g., horse). In some instances, the mammal is bovine (e.g., cow, buffalo, bison). In some instances, the mammal is a sheep. In some instances, the mammal is a goat). In some instances, the mammal is a pig. In some instances, the mammal is a rodent (e.g., mouse, rat, rabbit, guinea pig).

In some instances, a subject described herein is affected by a disease or a condition. Devices, systems, kits and methods disclosed herein may be used to test for the disease or condition, detect the disease or condition, and/or monitor the disease or condition. Devices, systems, kits and methods disclosed herein may be used to test for the presence of inherited traits, monitor fitness, and determine family ties.

Devices, systems, kits and methods disclosed herein may be used to test for, detect, and/or monitor cancer in a subject. Non-limiting examples of cancers include breast cancer, prostate cancer, skin cancer, lung cancer, colorectal cancer/colon cancer, bladder cancer, pancreatic cancer, lymphoma, and leukemia.

Devices, systems, kits and methods disclosed herein may be used to test for, detect, and/or monitor an immune disorder or autoimmune disorder in a subject. Autoimmune and immune disorders include, but are not limited to, type 1 diabetes, rheumatoid arthritis, psoriasis, multiple sclerosis, lupus, inflammatory bowel disease, Addison's Disease, Graves Disease, Crohn's Disease and Celiac disease.

Devices, systems, kits and methods disclosed herein may be used to test for, detect, and/or monitor a disease or condition that is associated with aging of a subject. Disease and conditions associated with aging include, but are not limited to, cancer, osteoporosis, dementia, macular degeneration, metabolic conditions, and neurodegenerative disorders.

Devices, systems, kits and methods disclosed herein may be used to test for, detect, and/or monitor a blood disorder. Non-limiting examples of blood disorders are anemia, hemophilia, blood clotting and thrombophilia. For example, detecting thrombophilia may comprise detecting a polymorphism present in a gene selected from Factor V Leiden (FVL), prothrombin gene (PT G20210A), and methylenetetrahydrofolate reductase (MTHFR).

Devices, systems, kits and methods disclosed herein may be used to test for, detect, and/or monitor a neurological disorder or a neurodegenerative disorder in a subject. Non-limiting examples of neurodegenerative and neurological disorders are Alzheimer's disease, Parkinson's disease, Huntington's disease, Spinocerebellar ataxia, amyotrophic lateral sclerosis (ALS), motor neuron disease, chronic pain, and spinal muscular atrophy. Devices, systems, kits and methods disclosed herein may be used to test for, detect, and/or monitor a psychiatric disorder in a subject and/or a response to a drug to treat the psychiatric disorder.

Devices, systems, kits and methods disclosed herein may be used to test for, detect, and/or monitor a metabolic condition or disease. Metabolic conditions and disease, include, but are not limited to obesity, a thyroid disorder, hypertension, type 1 diabetes, type 2 diabetes, non-alcoholic steatohepatitis, coronary artery disease, and atherosclerosis.

Devices, systems, kits and methods disclosed herein may be used to test for, detect, and/or monitor an allergy or intolerance to a food, liquid or drug. By way of non-limiting example, a subject can be allergic or intolerant to lactose, wheat, soy, dairy, caffeine, alcohol, nuts, shellfish, and eggs. A subject could also be allergic or intolerant to a drug, a supplement or a cosmetic. In some instances, methods comprise analyzing genetic markers that are predictive of skin type or skin health.

In some instances, the condition is associated with an allergy. In some instances, the subject is not diagnosed with a disease or condition, but is experiencing symptoms that indicate a disease or condition is present. In other instances, the subject is already diagnosed with a disease or condition, and the devices, systems, kits and methods disclosed herein are useful for monitoring the disease or condition, or an effect of a drug on the disease or condition.

Devices, systems, kits and methods disclosed herein may be used to test for, detect, and/or monitor a pregnancy. In some instances, the subject is a pregnant subject. in her first, second, or third trimester of pregnancy. In some instances, the pregnant subject is at fewer than about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 weeks gestation.

In some instances, the pregnant subject is about 2 weeks pregnant to about 42 weeks pregnant. In some instances, the pregnant subject is about 3 weeks pregnant to about 42 weeks pregnant. In some instances, the pregnant subject is about 4 weeks pregnant to about 42 weeks pregnant. In some instances, the pregnant subject is about 5 weeks pregnant to about 42 weeks pregnant. In some instances, the pregnant subject is about 6 weeks pregnant to about 42 weeks pregnant. In some instances, the pregnant subject is about 7 weeks pregnant to about 42 weeks pregnant. In some instances, the pregnant subject is about 8 weeks pregnant to about 42 weeks pregnant.

In some instances, the pregnant subject has reached at least about 5 weeks, at least about 6 weeks, at least about 7 weeks, or at least about 8 weeks of gestation. In some instances, the pregnant subject has reached at least about 5 to about 8 weeks of gestation. In some instances, the pregnant subject has reached at least about 5 to about 8, at least about 5 to about 12, at least about 5 to about 16, at least about 5 to about 20, at least about 6 to about 21, at least about 6 to about 22, at least about 6 to about 24, at least about 6 to about 26, at least about 6 to about 28, at least about 6 to about 9, at least about 6 to about 12, at least about 6 to about 16, at least about 6 to about 20, at least about 6 to about 21, at least about 6 to about 22, at least about 6 to about 24, at least about 6 to about 26, or at least about 6 to about 28 weeks of gestation. In some instances, the pregnant subject has reached at least about 7 to about 8, at least about 7 to about 12, at least about 7 to about 16, at least about 7 to about 20, at least about 7 to about 21, at least about 7 to about 22, at least about 7 to about 24, at least about 7 to about 26, at least about 7 to about 28, at least about 8 to about 9, at least about 8 to about 12, at least about 6 to about 16, at least about 8 to about 20, at least about 8 to about 21, at least about 6 to about 22, at least about 8 to about 24, at least about 8 to about 26, or at least about 8 to about 28 weeks of gestation. In some instances, gestation times are determined measuring from the first day of the last menstrual period.

Devices, systems, kits and methods disclosed herein are not limited to medical or health related applications. For example, devices, systems, kits and methods disclosed herein may be used in the field of forensics or to detect blood doping through blood transfusions.

### NUMBERED EMBODIMENTS

The disclosure is further understood through review of the numbered embodiments recited herein. 1. A device comprising: a sample purifier for removing a cell from a biological fluid sample to produce a cell-depleted sample; at least one of a detection reagent and a signal detector for detecting a plurality of biomarkers in the cell-depleted sample. 2. The device of embodiment 1, wherein the plurality of biomarkers comprises multiple cell-free DNA fragments. 3. The device of embodiment 2, wherein each of the multiple cell-free fragments comprise a region represented by a first sequence or a second sequence at least 90% homologous to the first sequence. 4. The device of embodiment 1, wherein the plurality of biomarkers are nucleic acids, and wherein the device comprises at least one nucleic acid amplification reagent and at least one oligonucleotide having a sequence corresponding to the target nucleic acid. 5. The device of embodiment 4, wherein the at least one nucleic acid amplification reagent comprises an oligonucleotide primer capable of amplifying a region of a chromosome having a first sequence that is similar to a second sequence in a genome of a subject, and wherein the first sequence is physically distant enough from the second sequence such that the first sequence is present on a first cell-free nucleic acid of the subject and the second sequence is present on a second cell-free nucleic acid of the subject. 6. The device of embodiment 5, wherein at least one of the first sequence and the second sequence is repeated at least five times in the genome of the subject. 7. The device of embodiment 5, wherein the first sequence and the second sequence are each at least 10 nucleotides in length. 8. The device of embodiment 5, wherein the first sequence is on a first chromosome and the second sequence is on a second chromosome. 9. The device of embodiment 5, wherein the first sequence and the second sequence are on the same chromosome but separated by at least 1 nucleotide. 10. The device of embodiment 5, wherein the first sequence and the second sequence are in functional linkage. 11. The device of embodiment 5, wherein the first sequence is at least 80% identical to the second sequence. 12. The device of embodiment 1, wherein the biomarker is a cell-free nucleic acid. 13. The device of embodiment 1, wherein the aggregate contains at least two biomarkers. 14. The device of embodiment 1, wherein the sample purifier comprises a filter. 15. The device of embodiment 14, wherein the sample purifier comprises a wicking material or capillary device for pushing the biological fluid through the filter. 16. The device of embodiment 14, wherein the filter has a pore size of about 0.05 microns to about 2 microns. 17. The device of embodiment 1, wherein the sample purifier comprises a binding moiety that binds a nucleic acid, protein, cell surface marker, or microvesicle surface marker in the fluid sample. 18. The device of embodiment 17, wherein the binding moiety comprises an antibody, antigen binding antibody fragment, a ligand, a receptor, a peptide, a small molecule, or a combination thereof. 19. The device of embodiment 17, wherein the binding moiety is capable of binding an extracellular vesicle, wherein the extracellular vesicle is released from a fetal cell or a placental cell of the female subject. 20. The device of embodiment 4, wherein the at least one nucleic acid amplification reagent comprises at least one isothermal amplification reagent. 21. The device of embodiment 20, wherein the at least one isothermal amplification reagent comprises a recombinase polymerase, a single-strand DNA-binding protein, a strand-displacing polymerase, or a combination thereof 22. The device of embodiment 1, wherein the signal detector comprises a solid support. 23. The device of embodiment 22, wherein the solid support is a column. 24. The device of embodiment 22, wherein the solid support comprises a binding moiety that binds the amplification product. 25. The device of embodiment 24, wherein the binding moiety is an oligonucleotide. 26. The device of embodiment 1, wherein the signal detector is a lateral flow strip. 27. The device of embodiment 26, wherein the detection reagent comprises a gold particle or a fluorescent particle. 28. The device of embodiment 1, wherein the sample purifier removes cells from blood, and the cell-depleted sample is plasma. 29. The device of embodiment 1, wherein the device is contained in a single housing. 30. The device of embodiment 1, wherein the device operates at room temperature. 31. The device of embodiment 4, wherein the device detects the amplification product within about five minutes to about twenty minutes of receiving the biological fluid. 32. The device of embodiment 1, comprising a transport or storage compartment. 33. The device of embodiment 32, wherein the transport or storage compartment comprises an absorption pad or a fluid container. 34. The device of embodiment 1, comprising a communication connection. 35. The device of embodiment 34, wherein the communication connection is a wireless communication system, a cable, or a cable port. 36. The device of embodiment 1, comprising a transdermal puncture device. 37. A method comprising: obtaining a fluid sample from a subject, wherein the volume of the biological sample is not greater than about 300 μL; contacting at least one cell free nucleic acid in the fluid sample with an amplification reagent and an oligonucleotide primer that anneals to a sequence corresponding to a sequence of interest; and detecting the presence or absence of an amplification product, wherein the presence or absence indicates a health status of the subject. 38. The method of embodiment 37, wherein the fluid sample is a blood sample. 39. The method of embodiment 38, wherein the volume of the blood sample is not greater than 120 μl. 40. The method of embodiment 37, wherein the fluid sample is a plasma sample from blood. 41. The method of embodiment 40, wherein the volume of the plasma sample is not greater than 50 μl. 42. The method of embodiment 40, wherein the volume of the plasma sample is between about 10 μl and about 40 μl. 43. The method of any one of embodiments 37 to 42, wherein obtaining comprises performing a finger prick. 44. The method of embodiment 43, comprising milking a pricked finger to increase blood that comes from the finger prick. 45. The method of embodiment 38, wherein obtaining the blood sample does not comprise performing a phlebotomy. 46. The method of embodiment 37, wherein the fluid sample is a urine sample. 47. The method of embodiment 37, wherein the fluid sample is a saliva sample. 48. The method of any one of embodiments 37-47, comprising removing at least one of a cell, a cell fragment, and a microparticle, from the fluid sample. 49. The method of embodiment 37, wherein the sample contains about 25 pg to about 250 pg of total circulating cell free DNA. 50. The method of embodiment 49, sample comprises cell free DNA fragments having a length of about 20 base pairs to about 160 base pairs in length. 51. The method of embodiment 37, wherein the sample contains about 5 to about 100 copies of a sequence of interest. 52. The method of embodiment 51, wherein the sequence of interest is at least 10 nucleotides in length. 53. The method of embodiment 51, wherein the 100 copies are at least 90% identical to one another. 54. The method of embodiment 37, wherein amplifying comprises isothermal amplification. 55. The method of embodiment 37, wherein amplifying occurs at room temperature. 56. The method of embodiment 37, wherein the method comprises incorporating a tag into the amplification product as the amplifying occurs, and wherein detecting the at least one amplification product comprises detecting the tag. 57. The method of embodiment 56, wherein the tag does not comprise a nucleotide. 58. The method of embodiment 57, wherein detecting the amplification product comprises contacting the amplification product with a binding moiety that is capable of interacting with the tag. 59. The method of embodiment 58, comprising contacting the amplification product with the binding moiety on a lateral flow device. 60. The method of embodiment 37, wherein the steps (a) through (c) are performed in less than fifteen minutes. 61. The method of embodiment 37, wherein the method is performed by the subject. 62. The method of embodiment 37, wherein the method is performed by an individual without receiving technical training for performing the method. 63. The method of embodiment 37, comprising obtaining, contacting, and detecting with a single handheld device. 64. The method of embodiment 63, wherein the subject performs the obtaining by pressing their skin against a transdermal puncture device of the handheld device. 65. The method of embodiment 64, wherein the subject presses their skin against the transdermal puncture device not more than once. 66. The method of embodiment 64, wherein the subject presses their skin against the transdermal puncture device not more than twice. 67. The method of embodiment 37, wherein the health status is selected from the presence and the absence of a pregnancy. 68. The method of embodiment 37, wherein the health status is selected from the presence and the absence of a neurological disorder, a metabolic disorder, a cancer, an autoimmune disorder, an allergic reaction, and an infection. 69. The method of embodiment 37, wherein the health status is a response to a drug or a therapy. 70. A device comprising: a sample purifier that removes a cell from a fluid sample of a female subject; at least one nucleic acid amplification reagent; at least one oligonucleotide comprising a sequence corresponding to a Y chromosome, wherein the at least one oligonucleotide and nucleic acid amplification reagent are capable of producing an amplification product; and at least one of a detection reagent or a signal detector for detecting the amplification product. 71. The device of embodiment 70, wherein the fluid sample is blood. 72. The device of embodiment 70, wherein the sample purifier comprises a filter. 73. The device of embodiment 72, wherein the sample purifier comprises a wicking material or capillary device for pushing the biological fluid through the filter. 74. The device of embodiment 72, wherein the filter has a pore size of about 0.05 microns to about 2 microns. 75. The device of embodiment 70, wherein the sample purifier comprises a binding moiety that binds a nucleic acid, protein, cell surface marker, or microvesicle surface marker in the fluid sample. 76. The device of embodiment 75, wherein the binding moiety comprises an antibody, antigen binding antibody fragment, a ligand, a receptor, a peptide, a small molecule, or a combination thereof. 77. The device of embodiment 76, wherein the binding moiety is capable of binding an extracellular vesicle, wherein the extracellular vesicle is released from a fetal cell or a placental cell of the female subject. 78. The device of embodiment 76, wherein the binding moiety binds a human chorionic gonadotropin protein or a transcript of a human chorionic gonadotropin encoding gene. 79. The device of embodiment 70, wherein the at least one oligonucleotide comprises a primer that hybridizes to a Y chromosome sequence. 80. The device of embodiment 70, wherein the at least one oligonucleotide comprises a probe that hybridizes to a nucleic acid represented by a Y chromosome sequence or transcript thereof, and wherein the probe comprises an oligonucleotide tag. 81. The device of embodiment 80, wherein the oligonucleotide tag is not specific to a Y chromosome sequence. 82. The device of embodiment 80 or 81, wherein the device comprises at least one primer that hybridizes to the oligonucleotide tag, and produces an amplification product in the presence of the amplification reagent. 83. The device of embodiment 80, wherein the Y chromosome sequence is a sequence located between position 20082183 and position 20350897 of the Y chromosome. 84. The device of embodiment 80, wherein the Y chromosome sequence is a sequence located between position 20350799 and position 20350897 of the Y chromosome. 85. The device of embodiment 80, wherein the Y chromosome sequence is a sequence located between position 20349236 and position 20349318 of the Y chromosome. 86. The device of embodiment 80, wherein the Y chromosome sequence is a sequence located between position 20082183 and position 20350897 of the Y chromosome. 87. The device of embodiment 80, wherein the Y chromosome sequence is a sequence located between position 20350601 and position 20350699 of the Y chromosome. 88. The device of embodiment 80, wherein the Y chromosome sequence is a sequence located between position 20082183 and position 20082281 of the Y chromosome. 89. The device of embodiment 80, wherein the Y chromosome sequence is a sequence located in a gene selected from DYS14 gene or a TTTY22. 90. The device of any one of embodiments 83 to 89, wherein the sequence is at least about 10 nucleotides in length. 91. The device of embodiment 70, wherein the at least one nucleic acid amplification reagent comprises at least one isothermal amplification reagent. 92. The device of embodiment 83, wherein the at least one isothermal amplification reagent comprises a recombinase polymerase, a single-strand DNA-binding protein, a strand-displacing polymerase, or a combination thereof. 93. The device of embodiment 70, wherein the signal detector comprises a solid support. 94. The device of embodiment 93, wherein the solid support is a column. 95. The device of embodiment 93, wherein the solid support comprises a binding moiety that binds the amplification product. 96. The device of embodiment 95, wherein the binding moiety is an oligonucleotide. 97. The device of embodiment 70, wherein the signal detector is a lateral flow strip. 98. The device of embodiment 97, wherein the detection reagent comprises a gold particle. 99. The device of embodiment 97, wherein the detection reagent comprises a fluorescent particle. 100. The device of embodiment 70, wherein the device is contained in a single housing. 101. The device of embodiment 70, wherein the device operates at room temperature. 102. The device of embodiment 70, wherein the device detects the amplification product within about five minutes to about twenty minutes of receiving the biological fluid. 103. The device of embodiment 70, comprising a transport or storage compartment. 104. The device of embodiment 103, wherein the transport or storage compartment comprises an absorption pad or a fluid container. 105. The device of embodiment 70, comprising a communication connection. 106. The device of embodiment 105, wherein the communication connection is a wireless communication system, a cable, or a cable port. 107. The device of embodiment 70, comprising a transdermal puncture device. 108. A kit comprising the device of any one of embodiments 70-107, and a component selected from a structure or reagent for obtaining a sample, purifying an analyte in the sample, amplifying the analyte, and detecting an analyte. 109. The kit of embodiment 108, wherein the component for obtaining a sample is a transdermal puncture device. 110. The kit of embodiment 109, comprising a capillary for drawing up blood from a transdermal puncture. 111. The kit of embodiment 108, comprising a container, pouch, wire or cable for heating or cooling the device of a component thereof 112. A method comprising: obtaining a fluid sample from a female pregnant subject, wherein the volume of the biological sample is not greater than about 300 μL; contacting at least one cell free nucleic acid in the fluid sample with an amplification reagent and an oligonucleotide primer that anneals to a sequence corresponding to a sex chromosome; and detecting the presence or absence of an amplification product, wherein the presence or absence indicates the gender of a fetus of the female pregnant subject. 113. The method of embodiment 112, wherein the fluid sample is a blood sample. 114. The method of embodiment 113, wherein the volume of the blood sample is not greater than 120 μl. 115. The method of embodiment 112, wherein the fluid sample is a plasma sample from blood. 116. The method of embodiment 115, wherein the volume of the plasma sample is not greater than 50 μl. 117. The method of embodiment 115, wherein the volume of the plasma sample is between about 10 μl and about 40 μl. 118. The method of any one of embodiments 112 to 117, wherein obtaining comprises performing a finger prick. 119. The method of embodiment 118, comprising milking a pricked finger to increase blood that comes from the finger prick. 120. The method of embodiment 113, wherein obtaining the blood sample does not comprise performing a phlebotomy. 121. The method of embodiment 112, wherein the fluid sample is a urine sample. 122. The method of embodiment 112, wherein the fluid sample is a saliva sample. 123. The method of any one of embodiments 112-122, comprising removing at least one of a cell, a cell fragment, and a microparticle, from the fluid sample. 124. The method of embodiment 112, wherein the sample contains about 25 pg to about 250 pg of total circulating cell free DNA. 125. The method of embodiment 112, wherein the cell free nucleic acid comprises a cell free fetal DNA fragment. 126. The method of embodiment 125, wherein the cell free fetal DNA fragment is about 20 base pairs to about 160 base pairs in length. 127. The method of embodiment 112, wherein the sequence corresponding to the sex chromosome is a Y chromosome sequence that is present in at least two copies on the Y chromosome. 128. The method of embodiment 127, wherein the Y chromosome sequence is a sequence located between position 20082183 and position 20350897 of the Y chromosome. 129. The method of embodiment 127, wherein the Y chromosome sequence is a sequence located between position 20350799 and position 20350897 of the Y chromosome. 130. The method of embodiment 127, wherein the Y chromosome sequence is a sequence located between position 20349236 and position 20349318 of the Y chromosome. 131. The method of embodiment 127, wherein the Y chromosome sequence is a sequence located between position 20082183 and position 20350897 of the Y chromosome. 132. The method of embodiment 127, wherein the Y chromosome sequence is a sequence located between position 20350601 and position 20350699 of the Y chromosome. 133. The method of embodiment 127, wherein the Y chromosome sequence is a sequence located between position 20082183 and position 20082281 of the Y chromosome. 134. The method of embodiment 127, wherein Y chromosome sequence is a sequence present in a DYS14 gene or a TTTY22 gene. 135. The method of any one of embodiments 127 to 134, wherein the sequence is at least about 10 nucleotides in length. 136. The method of embodiment 112, wherein the sample does not contain more than about 100 copies of the cell free nucleic acid. 137. The method of embodiment 112, wherein the sample contains about 5 to about 100 copies of the cell free nucleic acid. 138. The method of embodiment 112, wherein the female pregnant subject is not more than 8 weeks pregnant. 139. The method of embodiment 112, wherein amplifying comprises isothermal amplification. 140. The method of embodiment 112, wherein amplifying occurs at room temperature. 141. The method of embodiment 112, wherein amplifying comprises contacting the circulating cell free nucleic acid with a recombinase polymerase. 142. The method of embodiment 112, comprising tagging the cell free nucleic acid with an oligonucleotide tag. 143. The method of embodiment 112, wherein amplifying comprises contacting the cell free nucleic acid with at least one oligonucleotide primer having a sequence corresponding to the oligonucleotide tag. 144. The method of embodiment 143, wherein the oligonucleotide primer comprises a blocking group that prevents extension of the oligonucleotide primer until at least one of an amplification condition and amplification reagent is provided. 145. The method of embodiment 112, wherein the method comprises incorporating a tag into the amplification product as the amplifying occurs, and wherein detecting the at least one amplification product comprises detecting the tag. 146. The method of embodiment 145, wherein detecting the amplification product comprises detecting an amplified oligonucleotide tag. 147. The method of embodiment 145, wherein the tag comprises a nucleotide. 148. The method of embodiment 145, wherein the tag does not comprise a nucleotide. 149. The method of embodiment 145, wherein detecting the amplification product comprises contacting the amplification product with a binding moiety that is capable of interacting with the tag or oligonucleotide tag. 150. The method of embodiment 149, comprising contacting the amplification product with the binding moiety on a lateral flow device. 151. The method of embodiment 112, wherein the steps (a) through (c) are performed in less than fifteen minutes. 152. The method of embodiment 112, wherein the method is performed by the subject. 153. The method of embodiment 112, wherein the method is performed by an individual without receiving technical training to perform the method. 154. A method comprising: obtaining a fluid sample from a female pregnant subject with a handheld device, wherein the volume of the fluid sample is not greater than about 300 μL; sequencing at least one cell free nucleic acid in the fluid sample with the handheld device; detecting the presence or absence of a sequence corresponding to a Y chromosome through a display in the handheld device, thereby determining a gender of a fetus in the female pregnant subject; and communicating, with the handheld device, the gender to another subject. 155. The method of embodiment 154, wherein the detecting and communicating occur simultaneously. 156. The method of embodiment 154, wherein the volume is not greater than 120 μL. 157. The method of embodiment 154, wherein obtaining does not comprise a phlebotomy. 158. The method of embodiment 154, wherein the female pregnant subject performs the obtaining by pressing her skin against a transdermal puncture device of the handheld device. 159. The method of embodiment 158, wherein the female pregnant subject presses a finger against the transdermal puncture device. 160. The method of embodiment 158, wherein the female pregnant subject presses her skin against the transdermal puncture device not more than once. 161. The method of embodiment 158, wherein the female pregnant subject presses her skin against the transdermal puncture device not more than twice.

### EXAMPLES

The following examples are given for the purpose of illustrating various embodiments of the methods, devices, systems and kits disclosed herein and are not meant to limit the present methods, devices, systems and kits in any fashion. The present examples, along with the methods described herein are presently representative of preferred embodiments, are exemplary, and are not intended as limitations on the scope of the methods, devices, systems and kits disclosed herein. Changes therein and other uses which are encompassed within the spirit of the methods, devices, systems and kits disclosed herein as defined by the scope of the claims will occur to those skilled in the art.

### Example 1: Device for Analysis of Cell-Free Nucleic Acids from Whole Blood

A device for purifying separating plasma from maternal whole blood for the purpose of analyzing cell-free fetal nucleic acids was constructed. The device consists of 6 layers. From bottom to top these are:

(1) Lower Adhesive Sheet

(2) Lower Separation Disc: 16 mm diameter disc of adhesive sheet material (polymer material that is inert to DNA or Plasma) with glue on the side facing the Lower Adhesive Sheet

(3) Polyethersulfone (PES) membrane, various sizes, typically between 6 and 16 mm, preferred design features 10 mm PES membrane. The membrane serves as wicking material which attracts the plasma from the filter through capillary force.

(4) Filter Disc (e.g., Pall Vivid™ Membrane), 16 mm diameter, rough side facing up, shiny side facing the PES membrane.

(5) Upper Separation Disc: same material as Lower Separation Disc, size 12 or 14 mm diameter, containing a 4 mm hole in the center. When using adhesive sheet material, now the glue side is facing up to meet the Upper Adhesive Sheet. The Upper Separation Disc is smaller than the Filter Disc in diameter. This allows the glue from the Upper Adhesive Sheet to interact with the edges of the Filter Disc and thereby sealing it at the edges.

(6) Upper Adhesive Sheet, a 6 mm hole is punched in the location where the center of the device will be located.

All layers are lined up at their center and then laminated using a standard office lamination machine.

To evaluate the plasma transfer onto the PES membrane, the membrane was weighed before and after application of the plasma to the Disc Filter. The device construction was slightly altered to allow quick removal of the PES membrane. Instead of sandwiching the layers from Upper to Lower Separation Discs between Adhesive Sheets, a set of concentric spacer discs were applied to the top of the device, ensuring a tight fit between the filter and the PES membrane. The Lower Separation Disc was replaced with a parafilm layer. 80 μl of whole blood was applied to the center of the device through the hole in the Upper Adhesive Sheet and the hole in the Upper Separation Disc. This volume was chosen to maximize the amount of plasma transferred onto the PES membrane. However, a volume of plasma (0.5 μl to 1 μl) could have been obtained with 10 μl of blood and this would have been sufficient for Y chromosome detection. The blood distributed centripetally throughout the Filter Disc by capillary forces. Plasma was also wicked through the Filter Disc into the PES membrane by capillary forces. After about two minutes, an average of 6.3 μg of plasma was transferred to the PES membrane, indicating about 6 to 7 μl of plasma had been transferred to the PES membrane as shown in the following Table 2.

| Blood volume | Weight of the | Weight of the | |
|---|---|---|---|
| applied | PES/Lower | PES/Lower | μg of |
| to Vivid ™ | Disc after | Disc after | plasma in the |
| filter | filtration in μg | filtration in μg | PES membrane |
| 80 | 46.7 | 51 | 4.3 |
| 80 | 52 | 61 | 9 |
| 80 | 53.5 | 59.3 | 5.8 |
| 80 | 59 | 65.3 | 6.3 |
| Average | 52.8 | 59.15 | 6.35 |

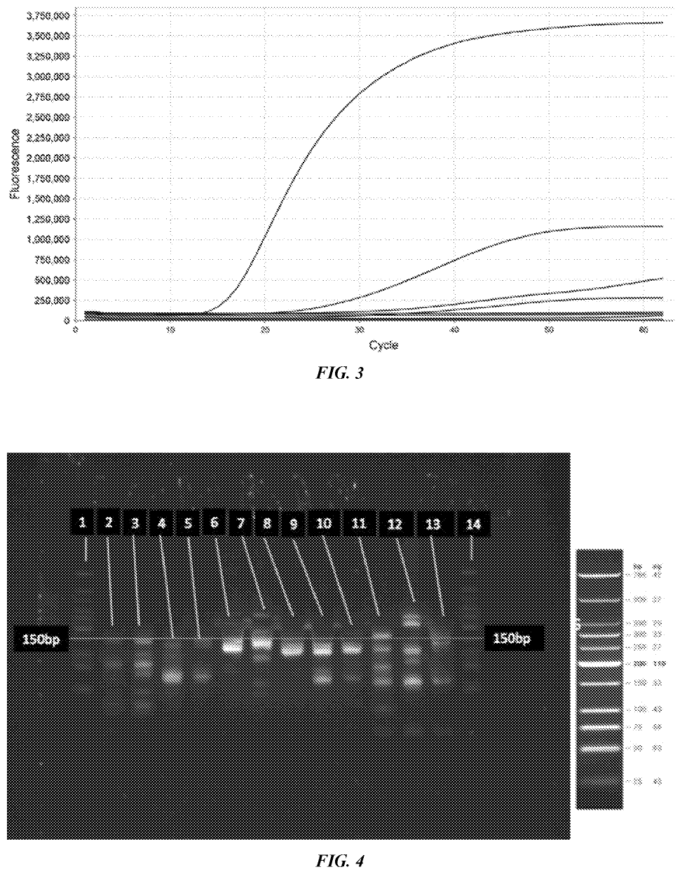

With the foregoing results taken in to account, 40 μl of male whole blood were transferred onto a device as described with a 12 mm Upper disc configuration. The PES membrane containing the plasma was transferred into an Eppendorf tube (0.5 ml) and 100 μl of EB buffer (QGEN) was added to elute the DNA on the PES membrane. After elution of the DNA from the membrane, 10 μl of the buffer containing the eluted cfDNA was used directly in a molecular amplification reaction. Real-time recombinase polymerase amplification was performed on the eluted cfDNA as described in Example 3 with primers specific to a marker on the Y chromosome. FIG. 3 shows a positive amplification of a Y chromosomal region starting around 12 minutes.

### Example 2: Device for Analysis of Fetal Cell-Free Nucleic Acids from Maternal Blood

The device consists of multiple layers as exemplified in Example 1.

Application of blood and filtration to the device occurs as follows:

40 μl to 60 μl of whole blood is applied to the center of the device through the hole in the Upper Adhesive Sheet and the hole in the Upper Separation Disc. The blood distributes centripetally throughout the Filter Disc by capillary forces. Plasma is also wicked through the Filter Disc into the PES membrane by capillary forces. After about two minutes, the maximum amount of plasma has been transferred into the PES membrane.

The PES membrane containing cell-free nucleic acids is recovered as follows:

The device is cut out around the edges of the PES membrane. The membrane separates easily from the Filter and the Lower Disc.

DNA is eluted from the membrane as follows:

The PES membrane containing the plasma is transferred into an Eppendorf tube (0.5 ml) and 100 μl of elution buffer are added (elution buffer can be H₂O, EB buffer (QGEN), PBS, TE or others suitable for subsequent molecular analysis). After elution of the DNA from the membrane, the buffer, containing the eluted cfDNA, is used directly in a molecular amplification reaction.

Amplification of eluted cfDNA and detection of a resulting amplification product is carried out according to a method in described in Example 3.

### Example 3. Detection of Human Y Chromosome DNA Using Recombinase Polymerase Amplification

Amplification and detection of human Y chromosome DNA in plasma samples were carried out by the following various methods:

Detection of Y Chromosome targets using RPA and polyacrylamide gel electrophoresis (PAGE):

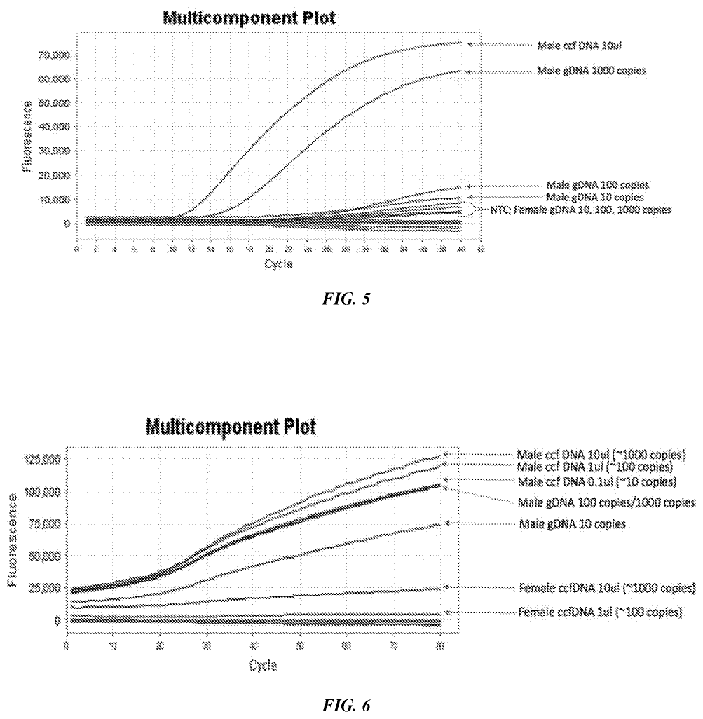

Recombinase polymerase amplification of 50 ng (15151 copies) of male genomic DNA was conducted using the TwistAmp Basic Kit (TwistDx, Cambridge, UK) following the standard protocol. Briefly, 29.5 μl of Rehydration Buffer was combined with 3 μl of each amplification primer (10 μM) (IDT, Coralville, Iowa), 2 μl of water and 10 μl of DNA template. 47.5 μl of this mixture was mixed with the lyophilized RPA enzymes as provided (uvsX, uvsY, gp32, Bsu). Following resuspension of the lyophilized RPA enzymes the reaction mixture was added to 2.5 μl of 280 mM magnesium acetate and mixed thoroughly to activate the RPA reaction. The reaction was incubated at 37 degrees Celsius for 20 minutes with agitation every 5 minutes to re-disperse the PEG crowding reagent. Immediately following RPA incubation products were purified using a Qiagen MinElute column (Qiagen Corporation, Valencia, Calif.) following the manufacturing's instructions with elution in 10 μl of Buffer EB. Purified RPA products were then subjected to 10% TBE PAGE using the Invitrogen NuPAGE Gel and the Mini Gel Tank System with 1×TBE as the running buffer (Thermo Fisher, Carlsbad, Calif.) at 150V (Constant) for approximately 1 hour. Gel lanes contained 41 of purified RPA product or Low Molecular Weight DNA Ladder (NEB, Ipswitch, Mass.), 6 μl of distilled water and 2 μl of 5× Novex loading dye (Thermo Fisher Scientific, Carlsbad, Calif.). Staining of gels was conducted using 20 μl of SyberSafe Dye in 200 ml of 1×TBE buffer (Thermo Fisher Scientific, Carlsbad, Calif.) for 20 minutes at room temperature with agitation. Following staining, gels were visualized and images captured using a blue light E-Gel Safe Imager Real-Time Transilluminator (Thermo Fisher Scientific, Carlsbad, Calif.). Primer sequences for specific RPA reactions are listed in Table 3. Results are shown in FIG. 4.

TABLE 3
Primers used to amplify Y chromosome sequences

| | | | | Detection |
|---|---|---|---|---|
| Gene | Oligo ID | Sequence (5′-3′) | Orientation | Method |
| TSPY1/ | DYS14_1_F_ | CTTCGGCCTTTCTAGTGGAGAGG | Sense | RPA, PCR, |
| DYS14 | Long | TGCTCTCG (SEQ ID NO. 6) | | RPA-Exo, |
| | | | | RPA-LF |
| TSPY1/ | DYS14_5_ | CCTGCTCCGGCTTTCCACAGCCA | Antisense | RPA, PCR, |
| DYS14 | R_Long | CACTGGT (SEQ ID NO. 7) | | RPA-Exo |
| TSPY1/ | DYS14_5_ | BioTEG- | Antisense | RPA-LF |
| DYS14 | R_long_LF_ | CCTGCTCCGGCTTTCCACAGCCA | | |
| | BioTEG | CACTGGT (SEQ ID NO. 8) | | |
| TSPY1/ | DYS14_10_ | ACCGATGGGCAGCTCGGCGTCG | Sense | RPA-Exo |
| DYS14 | Exo-P-1 | ATGTGACTCT[FAM][dSpacer]T | | |
| | | [BHQ1]GGGGAACAAAGGG-C3 | | |
| | | (SEQ ID NO. 9) | | |
| TSPY1/ | DYS14_10_ | FAM- | Sense | RPA-LF |
| DYS14 | LF-P-1 | ACCGATGGGCAGCTCGGCGTCG | | |
| | | ATGTGACTCT[dSpacer]TGGGGAA | | |
| | | CAAAGGG-C3 (SEQ ID NO. 10) | | |
| TSPY1/ | DYS14_5_F_ | TCTTTGGGGAACAAAGGGGAGT | Sense | RPA, PCR |
| DYS14 | long | TGCCACGG (SEQ ID NO. 11) | | |
| TSPY1/ | DYS14_4_ | CTTCTGCTCTTCAAAAAGATGCC | Antisense | RPA, PCR |
| DYS14 | R_long | CCAAACGT (SEQ ID NO. 12) | | |
| TSPY1/ | RPA_TSPY1_ | GAGCGGAAGAGGTTTTTCAGTG | Sense | RPA, PCR |
| DYS14 | 2_F2 | AATGAAGC (SEQ ID NO. 13) | | |
| TSPY1/ | RPA_TSPY1_ | GTCTGAGGAGTGGCAGAATCTG | Antisense | RPA, PCR |
| DYS14 | 2_R2 | CTTATAGC (SEQ ID NO. 14) | | |
| TSPY1/ | LF_TSPY1_ | Bio- | Sense | RPA-LF |
| DYS14 | 2_F2 | GAGCGGAAGAGGTTTTTCAGTG | | |
| | | AATGAAGC (SEQ ID NO. 15) | | |
| TSPY1/ | LF_TSPY1_ | DigN- | Antisense | RPA-LF |
| DYS14 | 2_R2 | GTCTGAGGAGTGGCAGAATCTG | | |
| | | CTTATAGC (SEQ ID NO. 16) | | |
| TSPY1/ | RPA_TSPY1_ | TTGTCCTGCATGCGGCAGAGAA | Sense | RPA, PCR |
| DYS14 | 3_F3 | ACCCTTGG (SEQ ID NO. 17) | | |
| TSPY1/ | RPA_TSPY1_ | ATAGCTTCATTCACTGAAAAAC | Antisense | RPA, PCR |
| DYS14 | 3_R3 | CTCTTCCG (SEQ ID NO. 18) | | |
| TTTY22 | TTTY22_1_ | GCTAATGTCTGTCCTCTCCTAGA | Sense | RPA, PCR |
| | F_long | ACTATGG (SEQ ID NO. 19) | | |
| TTTY22 | TTTY22_2_ | CTGCCATAAGGTAGAGAAGTAG | Antisense | RPA, PCR |
| | R_long | CCCTTCGT (SEQ ID NO. 20) | | |
| TTTY22 | TTTY22_7_ | CGCTAGGCAATGGTGGCATTCA | Sense | RPA, PCR |
| | F_long | TTGTGATGC (SEQ ID NO. 21) | | |
| TTTY22 | TTTY22_6_ | GACAGCTCTGACAACAGGACAC | Antisense | RPA, PCR |
| | R_long | CAGAGCCT (SEQ ID NO. 22) | | |
| TTTY22 | TTTY22_6_ | CCTGAGACTAGTGCATTGCATTG | Sense | RPA, PCR |
| | F-long | GTGAGGC (SEQ ID NO. 23) | | |
| TTTY22 | TTTY22_4_ | GCATCATTTTTTTTGACATCAGG | Antisense | RPA, PCR |
| | R_long | CCACTACTGC (SEQ ID NO. 24) | | |
| TTTY22 | TTTY22_12_ | ATATTTTCCTCTGTTTAGGAAGG | Antisense | RPA-Exo |
| | Exo_P-1 | CTGACAGCT[TAMRA][dSpacer]T | | |
| | | [BHQ2]GACAACAGGACACC | | |
| | | (SEQ ID NO. 25) | | |
| DYZ1 | RPA- | GTAGCATTCCACTTTATTCCAGG | Sense | RPA, PCR |
| | DYZ1-F1 | CCTGTCC (SEQ ID NO. 26) | | |
| DYZ1 | RPA- | AAGAGAATAGAATGGAATGCAA | Antisense | RPA, PCR |
| | DYZ1-R1 | GCGAAAGG (SEQ ID NO. 27) | | |
| DYZ1 | LF-DYZ1- | Bio- | Sense | RPA, PCR |
| | F1 | GTAGCATTCCACTTTATTCCAGG | | |
| | | CCTGTCC (SEQ ID NO. 28) | | |
| DYZ1 | LF-DYZ1- | 6FAM- | Antisense | RPA, PCR |
| | R1 | AAGAGAATAGAATGGAATGCAA | | |
| | | GCGAAAGG (SEQ ID NO. 29) | | |

TABLE 4
Amplicon Sequences for Y chromosome detection via
RPA or PCR

| Gene | Amplicon | Sequence (5′-3′) |
|---|---|---|
| TSPY1/ | DYS14- | CTTCGGCCTTTCTAGTGGAGAGGTGCTCTCGGGG |
| DYS14 | Amp10 | AAGTGTAAGTGACCGATGGGCAGCTCGGCGTCGA |
| | | TGTGACTCTTTGGGGAACAAAGGGGAGTTGCCAC |
| | | GGACCAGTGTGGCTGTGGAAAGCCGGAGCAGG |
| | | (SEQ ID NO. 30) |
| TSPY1/ | DYS14- | TCTTTGGGGAACAAAGGGGAGTTGCCACGGACCA |
| DYS14 | Amp11 | GTGTGGCTGTGGAAAGCCGGAGCAGGCGTGGGTA |
| | | CTATTGTCCTGCATGCGGCAGAGAAACCCTTGGT |
| | | GATGCCGAGCAGCAGACGTTTGGGGCATCTTTTT |
| | | GAAGAGCAGAAG (SEQ ID NO. 31) |
| TTTY22 | TTTY22- | GCTAATGTCTGTCCTCTCCTAGAACTATGGGAAT |
| | Amp10 | ATCCTGTGGACCCCACACAGAAGAAGGCAAGAAT |
| | | CCATGGTCTGTGCACCTCCACGAAGGGCTACTTC |
| | | TCTACCTTATGGCAG (SEQ ID NO. 32) |
| TTTY22 | TTTY22- | CGCTAGGCAATGGTGGCATTCATTGTGATGCTAG |
| | Amp 11 | CCAGAGCTCACAGCTCAGGCCTGGTGCCCTGAGA |
| | | CTAGTGCATTGCATTGGTGAGGCAGGCTCTGGTG |
| | | TCCTGTTGTCAGAGCTGTC (SEQ ID NO. 33) |
| TTTY22 | TTTY22- | CCTGAGACTAGTGCATTGCATTGGTGAGGCAGGC |
| | Amp 12 | TCTGGTGTCCTGTTGTCAGAGCTGTCAGCCTTCC |
| | | TAAACAGAGGAAAATATTATAGGCAGTAGTGGCC |
| | | TGATGTCAAAAAAAATGATGC (SEQ ID NO. 34) |

### Detection of Y Chromosome Targets Using RPA and Agarose Gel Electrophoresis:

Recombinase polymerase amplification was conducted using the TwistAmp Basic Kit (TwistDx, Cambridge, UK) following the standard protocol. Briefly, 29.5 μl of Rehydration Buffer is combined with 3 μl of each amplification primer (10 uM), 2 μl of water and 10 μl of DNA template. 47.5 μl of this mixture is mixed with the lyophilized RPA enzymes as provided (uvsX, uvsY, gp32, Bsu). Following resuspension of the lyophilized RPA enzymes the reaction mixture is added to 2.5 μl of 280 mM magnesium acetate and mixed thoroughly to activate the RPA reaction. Alternatively, RPA reactions were conducted using RPA enzymes (uvsX, uvsY, gp32, Bsu DNA Polymerase (large fragment) and buffers manufactured by New England Biolabs (Ipswich, Mass.). Here the following reagents were combined to a final concentration of: 2×NEB Buffer 4, 200 ng/μl uvsX, 40 ng/μl uvsY, 300 ng/μl gp32, 7.5U, 300 nM loci specific primers, 200 uM dNTPs (Life Technologies, Carlsbad, Calif.), 3 mM ATP, 50 mM Phosphocreatine (Sigma-Alrich), 100 ng/μl Creatine Kinase (Sigma-Alrich), 5% Polyethylene Glycol (Sigma-Aldrich) in a 50 μl reaction. Reactions were incubated at 37 degrees Celsius for 20 minutes with agitation every 5 minutes to re-disperse the PEG crowding reagent. RPA products were then purified using the MinElute Reaction Cleanup Kit (Qiagen Corporation, Valencia, Calif.) and subjected to 4% agarose gel electrophoresis using the Invitrogen E-Gel EZ and the E-Gel iBase (Thermo Fisher, Carlsbad, Calif.) for 15 minutes. Gel lanes contained 5-10 μl of purified RPA product or Low Molecular Weight 25 bp DNA Ladder (Thermo Fisher, Carlsbad, Calif.) 8-13 μl of distilled water and 41 of 5× Qiagen loading dye (Qiagen Corporation, Valencia, Calif.). Gels were visualized and images captured using a blue light E-Gel Safe Imager Real-Time Transilluminator (Thermo Fisher Scientific, Carlsbad, Calif.). Primer sequences for specific RPA reactions are listed in Table 3.

FIG. 9 shows a 4% agarose gel with RPA products generated for the TSPY1 (DYS14) loci on the Y chromosome using NEB manufactured enzymes, self-assembled ATP regeneration reagents and PEG. Loading was as follows:

### Lane M: Ladder

Lane 1: RPA-DYS14-3 (118 bp)—NTC
Lane 2: RPA-DYS14-3 (118 bp)—Female gDNA (Promega)
Lane 3: RPA-DYS14-3 (118 bp)—Female gDNA (Zyagen)
Lane 4: RPA-DYS14-3 (118 bp)—Female ccfDNA
Lane 5: RPA-DYS14-3 (118 bp)—Male gDNA (Promega)
Lane 6: RPA-DYS14-3 (118 bp)—Male gDNA (Zyagen)
Lane 7: RPA-DYS14-3 (118 bp)—Male ccfDNA
The gel clearly show that the expected product is only present in reactions containing male DNA and thus the Y chromosomal target for the RPA reaction.

### Detection of Y Chromosome Targets Using Real-Time RPA (RPA-Exo):

Recombinase polymerase amplification was conducted using the TwistAmp Exo Kit (TwistDx, Cambridge, UK) following the standard protocol. Briefly, 29.5 μl of Rehydration Buffer is combined with 2.1 μl of each amplification primer (10 uM), 0.6 μl of probe primer (10 uM), 2 μl of water and 10 μl of DNA template. 47.5 μl of this mixture is mixed with the lyophilized RPA enzymes as provided (uvsX, uvsY, gp32, Bsu, Exonuclease III). Following resuspension of the lyophilized RPA enzymes the reaction mixture is added to 2.5 μl of 280 mM magnesium acetate and mixed thoroughly to activate the RPA reaction. The reaction is incubated at 37 degrees Celsius for 20+ minutes in a OneStep Real-Time PCR Cycler (Thermo Fisher Scientific, Carlsbad, Calif.) and the data analyzed with StepOne Software v2.3 (Thermo Fisher Scientific, Carlsbad, Calif.). Primer sequences for specific RPA real time reactions are listed in Table 3. The probe primer used for real-time detection has an internal fluorophore (e.g. FAM) which is separated from a Black Hole Quencher (BHQ) moiety by an abasic site and a 3′ cap to prevent extension during RPA. The FAM fluorophore is quenched when in proximity to the BHQ. When the probe is bound to its template Exonuclease III can cleave the abasic site and the BHQ is subsequently released resulting in FAM fluorescence which is read on the real-time cycler.

As shown in FIG. 5, human Y chromosome can be detected using circulating cell-free DNA isolated from male donor whole blood as can male genomic DNA at varying copy levels down to 10 copies with a corresponding increase in signal with increased input copies. Fluorescent signal of an internal FAM-labeled hybridization probe is shown as a function of fluorescent signal vs. time (cycle). Red lines represent ROX signal. Since RPA is an isothermal reaction cycles were defined as 30 second intervals for the collection of fluorescent signal on a OneStep real-time PCR cycler. Incubation was conducted at 37 degrees Celsius for 20 minutes which shows up on the X-axis as 40 cycles. As the figure shows, increased FAM-labeled probe binding, cleavage and signal (Y-axis) occurs between 5-20 minutes for RPA reactions containing human Y chromosome template. Negative control template (water) and female genomic DNA both showed no detection as expected.

The ability to detect human Y chromosome via the DYS14 loci in ccfDNA using RPA was further evaluated by serial diluting the input amount of ccfDNA from approximately 1000 copies down to 10 copies. As shown in FIG. 6 the FAM-based fluorescent signal increases with increased input amount of male human ccfDNA and male human gDNA. Fluorescent signal of an internal FAM-labeled hybridization probe is shown as a function of fluorescent signal vs. time (cycle). The bottom two lines (flat) represent ROX signal. Since RPA is an isothermal reaction cycles were defined as 30 second intervals for the collection of fluorescent signal on a OneStep real-time PCR cycler. Incubation was conducted at 37 degrees Celsius for 40 minutes which shows up on the X-axis as 80 cycles. As the figure shows, increased FAM-labeled probe binding, cleavage and signal (Y-axis) occurs for RPA reactions containing increasing amounts of human male ccfDNA template. The signal for ccfDNA is stronger than that of gDNA perhaps due to greater accessibility for the recombinase in the highly fragmented ccfDNA. Female ccfDNA showed no signal at an input level of 100 copies and a very low signal at 1000 input copies. Human male Y chromosome is clearly detected in a specific manner using RPA with ccfDNA and gDNA via real-time detection.

### Detection of Y Chromosome Targets Using RPA and Lateral Flow (RPA-LF):

Recombinase polymerase amplification was conducted using the TwistAmp® nfo Kit (TwistDx, Cambridge, UK) following the standard protocol. Briefly, 29.5 μl of Rehydration Buffer is combined with 2.1 μl of each amplification primer (10 uM), 0.6 μl of probe primer (10 uM), 2 μl of water and 10 μl of DNA template. 47.5 μl of this mixture is mixed with the lyophilized RPA enzymes as provided (uvsX, uvsY, gp32, Bsu, Endonuclease IV(nfo)). Following resuspension of the lyophilized RPA enzymes the reaction mixture is added to 2.5 μl of 280 mM magnesium acetate and mixed thoroughly to activate the RPA reaction. Alternatively, RPA reactions were conducted using RPA enzymes (uvsX, uvsY, gp32, Bsu DNA Polymerase (large fragment) and buffers manufactured by New England Biolabs (Ipswich, Mass.). Here the following reagents were combined to a final concentration of: 2×NEB Buffer 4, 200 ng/μl uvsX, 40 ng/ul uvsY, 300 ng/μl gp32, 7.5U, 300 nM loci specific primers, 200 uM dNTPs (Life Technologies, Carlsbad, Calif.), 3 mM ATP, 50 mM Phosphocreatine (Sigma-Alrich), 100 ng/μl Creatine Kinase (Sigma-Alrich), 5% Polyethylene Glycol (Sigma-Aldrich) in a 50 μl reaction. The reaction is incubated at 37 degrees Celsius for 20 minutes with agitation every 5 minutes to re-disperse the PEG crowding reagent. Immediately following RPA incubation, products were visualized with a nucleic acid lateral flow immunoassay (NALFIA) e.g., the HybriDetect 2T lateral flow kit (Milenia Biotec, Giessen, Germany) or the PCRD Nucleic Acid Detector lateral flow kit (Abingdon, York, Great Britain). For the Hybridetect 2T strips, RPA products were diluted 1:50 in Assay Buffer 2, 10 μl of the diluted RPA product was then applied to the lateral flow strip and the strip incubated in 200 μl of Assay Buffer 2 for 5 minutes for visualization of the FAM/Biotin labeled RPA product. For the PCRD strips, 5 μl of RPA product was mixed with 70 μl of PCRD Extraction Buffer and then 75 μl applied directly to the detector. Products were then visualized between 2-5 minutes. Primer sequences for specific RPA lateral flow reactions are listed in Table 3. Lateral flow detection of the RPA products as such requires labeling of one of the amplification primers with Biotin along with labeling of a hybridization primer (probe) with an antigenic moiety (FAM or DIG in these cases). The probe contains an internal abasic site (dSPacer here) and is 3′-capped (C3 spacer here) in order to prevent extension until hybridization with the desired RPA product occurs; this, in theory, adds specificity to the reaction. However, highly specific products allow for labeling of the amplification primers without the need for an internal probe.

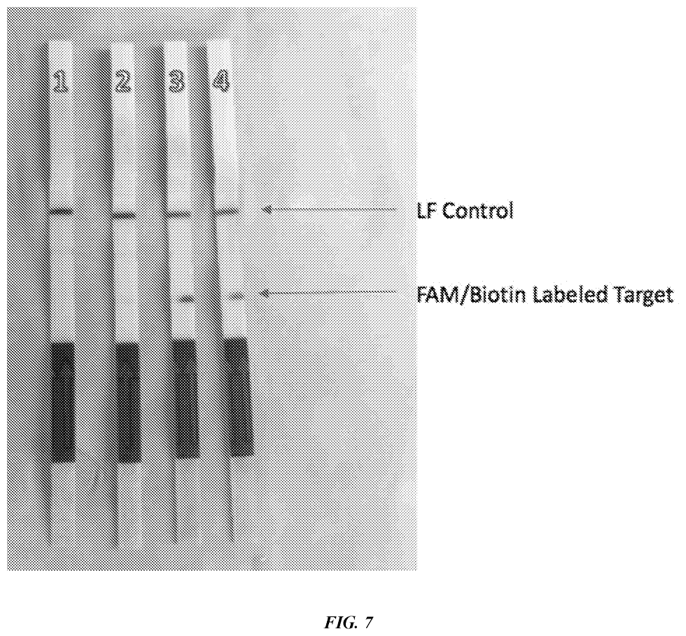

FIG. 7 shows lateral flow strips with human DYS14 Y chromosome RPA-LF products applied to the lateral flow strips at 5-minute time intervals from 0-20 minutes in order to determine if and when a Y chromosome signal appears. Lateral flow test strips (membranes coated with biotin-ligand and anti-FITC antibody in gold conjugate) from the HybriDetect 2T are shown. The complexed RPA analyte which is labeled with FAM and biotin binds first to the gold-labeled FITC-specific antibodies in the sample application of the test strip (bottom portion) and then diffuse over the membrane by capillary forces. The analyte captured gold particles bind when they overflow the immobilized biotin-ligand molecules at the respective test band location and generate a red-blue band over time (lower band). Non-captured gold particles flow over the upper control band and will be fixed there by species-specific antibodies (Upper band). With increasing incubation time, the formation of an intensely colored control band appears. All test strips were incubated for 5 minutes in Assay Buffer (Tris-buffered saline) following the application of 10 μl of RPA-LF product. The RPA reaction incubation times were as follows: Strip 1: 0 minutes; Strip 2: 5 minutes; Strip 3: 10 minutes; Strip 4: 20 minutes. As can be seen, a DYS14 specific (FAM/Biotin captured) product appears at 10 minutes and is also present at 20 minutes. No signal is present at time 0 or time 5 minutes whereas the lateral flow control signal is present on all strips demonstrating functionality.

FIG. 10 shows PCRD lateral flow strips with human TSPY1 (DYS14) Y chromosome RPA-LF products using NEB manufactured enzymes, self-assembled ATP regeneration reagents and PEG. The primers were labeled with Digoxigenin (DIG) and Biotin for capture and detection. The line at the “C” annotation represents the binding to the control conjugate and is expected are all samples. DIG conjugate binds in proximity to the “1” annotation and FAM the “2” annotation. From top to bottom the lateral flow detectors are:

Top: DYS14 Bio/DIG—NTC

Middle: DYS14 Bio/DIG—Female gDNA

Bottom: DYS14 BioDIG Male gDNA

The lateral flow detectors clearly demonstrate specific binding of labeled DYS14 RPA product labeled with Biotin and Digoxigenin.

### Example 4. Select Assay Designs for Y-Chromosome Region

TABLE 5
Assay Sequences from the highly repetitive Y-chromosome
region (HRYR):

| Assay | | | Consensus Target Sequence |
|---|---|---|---|
| ID | Sense Primer | Antisense Primer | (Amplicon) |
| Seq01 | GAATTCATTGG | ATTCCATACATT | TGGAATTCATTGGAATGGAAGGG |
| | AAGGAATGTA | TTTATTCCATTC | AATGTAGTGTAATGGACAGGCCTG |
| | GTGTAATGG | GAGACC (SEQ ID | GAATAAAGTGGAATGCTACGGTCT |
| | (SEQ ID NO. 37) | NO. 38) | CGAATGGAATAAAAATGTATGGA |
| | | | AT (SEQ ID NO. 141) |
| Seq02 | AATCGAATGG | TTACATTCTACT | TAATCGAATGGAAAGTAATCCAAT |
| | AAAGATCCAA | CTATCTGAGTCG | GGAATAGAATCTAATGCAATAAA |
| | TGGAATAGAA | ATTTTA (SEQ ID | ATCGACTCAGATAGAGTAGAATGT |
| | (SEQ ID NO. 39) | NO. 40) | AATGGAAT (SEQ ID NO. 142) |
| Seq03 | AGTAATCCAA | CATTACATTCTA | CGAATGGAAAGTAATCCAATGGA |
| | TGGAATAGAT | CTCTATCTGAGT | ATAGATTCTAATGCAATAAAATCG |
| | TCTAATGCAA | CGATTT (SEQ ID | ACTCAGATAGAGTAGAATGTAATG |
| | (SEQ ID NO. 41) | NO. 42) | GAAT (SEQ ID NO. 143) |
| Seq04 | AAACGGAATG | GATTCAATTCCA | CTGGAATCAAACGGAATGGAATG |
| | GAATGTAGTG | TTTGATTCTCTT | TAGTGCAATCAAATGGCATGGAAT |
| | CAATCAAATG | TCATTC (SEQ ID | AAAATAGAATGAAAGAGAATCAA |
| | (SEQ ID NO. 43) | NO. 44) | ATGGAATTGAATCGA |
| | | | (SEQ ID NO. 144) |
| Seq05 | AATGGAAAGG | CATTTGATCCTA | AATGGAAAGGACTCGAATGGAAA |
| | ACTCGAATGG | TTTTATTAAATT | TCACTCGAATAGAATGCAATTTAA |
| | AAATCACTCG | GCATTC (SEQ ID | TAAAATAGGATCAAATGTAATGG |
| | (SEQ ID NO. 45) | NO. 46) | AATG (SEQ ID NO. 145) |
| Seq06 | ATTGGATGGG | ATTCCATTCCGT | AGATGGGATTGGATGGGATTGGA |
| | ATTGGAATGA | TTCATGAAATTC | ATGAAATGTACTGGAAAGGACTC |
| | AATGTACTGG | GAGTCC (SEQ ID | GAATTTCATGAAACGGAATGGAAT |
| | (SEQ ID NO. 47) | NO. 48) | GAATTG (SEQ ID NO. 146) |
| Seq07 | AATGAACTCCT | ATTACATTCCTT | AGAATGGAATGAACTCCTTTGGAA |
| | TTGGAATGGT | TTGATTCCCTGC | TGGTGTAGTATGCAATGCAATCGA |
| | GTAGTATGC | CAGTCG (SEQ ID | CTGGCAGGGAATCAAAAGGAATG |
| | (SEQ ID NO. 49) | NO. 50) | TAATC (SEQ ID NO. 147) |
| Seq08 | ATGGAATGCA | GAGTCAATTCCT | TGTCATAGAATGTAATGGAATGCA |
| | AAAAAATGGA | TTCGACACCCA | AAAAAATGGAATCCAAAATCATT |
| | ATCCAAAATC | GCCTTTC (SEQ | GACTGGAAAGGCTGGGTGTCGAA |
| | (SEQ ID NO. 51) | ID NO. 52) | AGGAATTGACTCCAATGGAA (SEQ |
| | | | ID NO. 148) |
| 21255 | AATGGACAGG | ATTCCATTCCAT | AATGGACAGGCCTGGAATAAAGT |
| | CCTGGAATAA | ACATTTTTATTC | GGAATGCTACGGTCTCGAATGGAA |
| | AGTGGAATGC | CATTCG (SEQ ID | TAAAAATGTATGGAATGGAATGC |
| | (SEQ ID NO. 53) | NO. 54) | AAT (SEQ ID NO. 149) |
| 20202 | AATGGAATGT | ATTCCATTGGA | AATGGAATGTACTCGAATGGATTC |
| | ACTCGAATGG | GTCCATTCACTT | GACTGGAATGGAATGTTCTGGAAG |
| | ATTCGACTGG | CCAGAAC (SEQ | TGAATGGACTCCAATGGAATGGAT |
| | (SEQ ID NO. 55) | ID NO. 56) | T (SEQ ID NO. 150) |
| 19805 | GATGGACTGG | CATTCTATTTTA | GAACTTTCTTGGATGGACTGGAAT |
| | AATCAAACGG | TTCCATGCCATT | CAAACGGAATGGAATGCARTGCA |
| | AATGGAATGC | TGATTG (SEQ ID | ATCAAATGGCATGGAATAAAATA |
| | (SEQ ID NO. 57) | NO. 58) | GAATGAAAGAGAAT |
| | | | (SEQ ID NO. 151) |
| 18104 | AATGGATTGG | AGGCCTGTCCA | AATGGATTGGAATGGAATGGAATT |
| | AATGGAATGG | TTACACTACATT | CATTGGAATGGAAGGGAATGTAG |
| | AATTCATTGG | CCCTTCC (SEQ | TGTAATGGACAGGCCTGGAAT |
| | (SEQ ID NO. 59) | ID NO. 60) | (SEQ ID NO. 152) |
| 17131 | ATATAATGGA | TTTCCATTCCAT | AGTGGAGTGGACTCGAATATAATG |
| | CTGGAATGGA | TCCATTCGTTCC | GACTGGAATGGAATGAAATCACA |
| | ATGAAATCAC | CATTCC (SEQ ID | TGGAATGGGAACGAATGGAATGG |
| | (SEQ ID NO. 61) | NO. 62) | AATGGAAA (SEQ ID NO. 153) |
| 15595 | AATCGTATGG | ATTCGAGTGCA | AATCGTATGGAATGGCATCAAACG |
| | AATGGCATCA | TTCCATTCCGTG | GAATGGAATGGACAGCCACGGAA |
| | AACGGAATGG | GCTGTCC (SEQ | TGGAATGCACTCGAATGCAAT |
| | (SEQ ID NO. 63) | ID NO. 64) | (SEQ ID NO. 154) |
| 21099 | AATGGATTGG | AGGCCTGTCCA | AATGGATTGGAATGGAATGGAATT |
| | AATGGAATGG | TTACACTACATT | CATTGGAATGGAAGGGAATGTAG |
| | AATTCATTGG | CCCTTCC (SEQ | TGTAATGGACAGGCCTGGAA (SEQ |
| | (SEQ ID NO. 65) | ID NO. 66) | ID NO. 155) |
| 14192 | AATGGAATTG | GTTTGATTCCAT | AATGGAATGGAATTGAATGGAAA |
| | AATGGAAAGT | TCCGTGAAATTT | GTAATGCAATGGAATAGAATGGA |
| | AATGCAATGG | CGTTCC (SEQ ID | ACGAAATTTCACGGAATGGAATCA |
| | (SEQ ID NO. 67) | NO. 68) | AAC (SEQ ID NO. 156) |
| n-mer2 | AATGGAAGGG | ATACATTTTTAT | AATGGAAGGGAATGTAGTGTAAT |
| | AATGTAGTGT | TCCATTCGAGA | GGACAGGCCTGGAATAAAGTGGA |
| | AATGGACAGG | CCGTAGC (SEQ | ATGCTACGGTCTCGAATGGAATAA |
| | (SEQ ID NO. 69) | ID NO. 70) | AAATGTAT (SEQ ID NO. 157) |
| n-mer11 | AAAATCATTG | TATCAATTCCAT | AAAATCATTGACTGGAAAGGCTG |
| | ACTGGAAAGG | TCCATTCGATTT | GGTGTCGAAAGGAATTGACTCCAA |
| | CTGGGTGTCG | AGTTCG (SEQ ID | TGGAATGGAATCGAATGGAATGG |
| | (SEQ ID NO. 71) | NO. 72) | AAGTGAATAGAATCGAACTAAAT |
| | | | CGAATGGAATGGAATTGATA |
| | | | (SEQ ID NO. 158) |
| n-mer22 | ACTAGAGTGA | AGTGCATTCCAT | ACTAGAGTGAAATGGAATCGAAC |
| | AATGGAATCG | TCCGTGGCTGTC | CACAAGGAATGGACAGGAATAGA |
| | AACCACAAGG | CATTCC (SEQ ID | ATGGTCTCGAATTGAATGGAATCG |
| | (SEQ ID NO. 73) | NO. 74) | TATGGAATGGCATCAAACGGAAT |
| | | | GGAATGGACAGCCACGGAATGGA |
| | | | ATGCACT (SEQ ID NO. 159) |
| n-mer30 | GCATCAAACG | ATTCCATTCCAT | GCATCAAACGGAATGGAATGGAC |
| | GAATGGAATG | TGGAGTCCGTA | AGCCACGGAATGGAATGCACTCG |
| | GACAGCCACG | CCAGTCG (SEQ | AATGCAATGGAGTCGAAACTAAT |
| | (SEQ ID NO. 75) | ID NO. 76) | GGACTGGAATAGAATGGACTCGA |
| | | | CTGGTACGGACTCCAATGGAATGG |
| | | | AAT (SEQ ID NO. 160) |
| n-mer43 | AATAGAATGG | AGACCTTTCCAT | AATAGAATGGACTCGACTGGTACG |
| | ACTCGACTGGT | TGCAGTCTTTTC | GACTCCAATGGAATGGAATCGAAT |
| | ACGGACTCC | CCTTCG (SEQ ID | GGAAGGGAATCGAACGGAAKGGA |
| | (SEQ ID NO. 77) | NO. 78) | ATCGAACGGAATGGACTCGAAGG |
| | | | GAAAAGACTGCAATGGAAAGGTC |
| | | | T (SEQ ID NO. 161) |
| n-mer52 | AATGCATTGG | ATTCCAGTATAT | AATGCATTGGAATGGAATGTCCTC |
| | AATGGAATGT | TCCATTGTATTC | TAATGGAATGGATTCGAGTGGAAT |
| | CCTCTAATGG | GATCCC (SEQ ID | GGAATTGAATATAATGGAGTCGA |
| | (SEQ ID NO. 79) | NO. 80) | ATGAAATGGAATTGAAAGGAATG |
| | | | GGATCGAATACAATGGAATATACT |
| | | | GGAAT (SEQ ID NO. 162) |
| n-mer57 | AATGGGCTGG | AGTGCATTCCAT | AATGGGCTGGAATGGAAAGGAAT |
| | AATGGAAAGG | TCCAGTCTCTTC | CGAAACGAATGGAATGGAATCGA |
| | AATCGAAACG | AGTTCG (SEQ ID | ACTGAAGAGACTGGAATGGAATG |
| | (SEQ ID NO. 81) | NO. 82) | CACT (SEQ ID NO. 163) |
| n-mer66 | AGTGGAATGG | CATTCCAGTATA | AGTGGAATGGAATTGAATATAATG |
| | AATTGAATAT | TTCCATTGTATT | GAGTCGAATGAAATGGAATTGAA |
| | AATGGAGTCG | CGATCC (SEQ ID | AGGAATGGGATCGAATACAATGG |
| | (SEQ ID NO. 83) | NO. 84) | AATATACTGGAATG |
| | | | (SEQ ID NO. 164) |
| n-merP3-1 | ATGGGCTGGA | GTGCATTCCATT | ATGGGCTGGAATGGAAAGGAATC |
| | ATGGAAAGGA | CCAGTCTCTTCA | GAAACGAATGGAATGGAATCGAA |
| | ATCGAAACG | GTTCG (SEQ ID | CTGAAGAGACTGGAATGGAATGC |
| | (SEQ ID NO. 85) | NO. 86) | AC (SEQ ID NO. 165) |
| n-merP3-2 | AAGGAATGGA | TTCCATTCCGTT | AAGGAATGGAATCGAATGGCAAG |
| | ATCGAATGGC | CCGTTCACATCA | AAATCGAATGTAATGGAATCGCCA |
| | AAGAAATCG | ATTCC (SEQ ID | GGAATTGATGTGAACGGAACGGA |
| | (SEQ ID NO. 87) | NO. 88) | ATGGAAT (SEQ ID NO. 166) |
| n-merP3-3 | AGTGGAATGG | CATTCCAGTATA | AGTGGAATGGAATTGAATATAATG |
| | AATTGAATAT | TTCCATTGTATT | GAGTCGAATGAAATGGAATTGAA |
| | AATGGAGTCG | CGATCC (SEQ ID | AGGAATGGGATCGAATACAATGG |
| | (SEQ ID NO. 89) | NO. 90) | AATATACTGGAATG (SEQ ID NO. |
| | | | 167) |
| n-merP3-4 | AAACGGAATC | ACACCCAGCCT | AGTGGAATGGAATTGAATATAATG |
| | GAATGTCATA | TTCCAGTCAATG | GAGTCGAATGAAATGGAATTGAA |
| | GAATGTAATG | ATTTTGG (SEQ | AGGAATGGGATCGAATACAATGG |
| | G (SEQ ID NO. | ID NO. 92) | AATATACTGGAATG (SEQ ID NO. |
| | 91) | | 168) |
| n-merP3-5 | ATGGAAAGGA | CTTTCCATTCCA | ATGGAAAGGACTCGAATGGAAAT |
| | CTCGAATGGA | TTCCATTACATT | CACTCGAATAGAATGCAATTTAAT |
| | AATCACTCG | TGATCC (SEQ ID | AAAATAGGATCAAATGTAATGGA |
| | (SEQ ID NO. 93) | NO. 94) | ATGGAATGGAAAG (SEQ ID NO. |
| | | | 169) |
| u15pp005 | AATGGGCTGG | AGTGCATTCCAT | AATGGGCTGGAATGGAAAGGAAT |
| | AATGGAAAGG | TCCAGTCTCTTC | CGAAACGAATGGAATGGAATCGA |
| | AATCGAAACG | ASTTCG (SEQ ID | ACTGAAGAGACTGGAATGGAATG |
| | (SEQ ID NO. 95) | NO. 96) | CACT (SEQ ID NO. 170) |
| u15pp001 | AATGGAATGG | ATTCCATTCTAT | AATGGAATGGAATGGAAAGGAAT |
| | AATGGAAAGG | ACCATTGCTCTC | CGAAACGAAAGGAATGGAGACAG |
| | AATCGAAACG | TGTTCC (SEQ ID | ATGGAATGGAATGGAACAGAGAG |
| | (SEQ ID NO. 97) | NO. 98) | CAATGGTATAGAATGGAAT (SEQ |
| | | | ID NO. 171) |
| u15pp004 | AATGGRMTGG | ATTCMATTYCA | AATGGGCTGGAATGGAAAGGAAT |
| | AATGGAAAGG | KTCYCTTCMMT | CGAAACGAATGGAATGGAATCGA |
| | AATSGAAWCG | TCGATTCC (SEQ | ACTGAAGAGACTGGAATGGAAT |
| | (SEQ ID NO. 99) | ID NO. 100), | (SEQ ID NO. 172) |
| | | wherein M = A or C; | |
| | | K = G or T; and | |
| | | Y = C or T. | |
| u15pp024 | AATGGGAACG | TTCCATTCCAAT | AATGGGAACGAATGGAGTGAAAT |
| | AATGGAGTGA | CCATTCCTTTCC | TGTATGCAGTAGAAGAGAATAGA |
| | AATTGTATGC | TTTCGC (SEQ ID | ATGGAATGCAAGCGAAAGGAAAG |
| | (SEQ ID NO. | NO. 102) | GAATGGATTGGAATGGAA |
| | 101) | | (SEQ ID NO. 173) |
| u15pp021 | AATGGGAACG | AGTCCGTTCCAT | ATAAAATGGAAGAAAACTGGCAA |
| | AATGGAGTGA | AACACTCCATTC | GAAATGGAATCGAAATGAATGGA |
| | AATTGTATGC | ATTTCG (SEQ ID | GTGTTATGGAACGGACT |
| | (SEQ ID NO. | NO. 104) | (SEQ ID NO. 174) |
| | 103) | | |
| u15pp022 | ATAAAATGGA | ATTACATTCAAT | ATAAAATGGAAGAAAACTGGCAA |
| | AGAAAACTGG | TCCTTTTGAGTC | GAAATGGAATCGAAATGAATGGA |
| | CAAGAAATGG | CGTTCC (SEQ ID | GTGTTATGGAACGGACTCAAAAG |
| | (SEQ ID NO. | NO. 106) | GAATTGAATGTAAT |
| | 105) | | (SEQ ID NO. 175) |
| u15pp019 | CGGAATGGAA | AGTCCGTTCCAT | CGGAATGGAATAAAATGGAAGAA |
| | TAAAATGGAA | AACACTCCATTC | AACTGGCAAGAAATGGAATCGAA |
| | GAAAACTGGC | ATTTCG (SEQ ID | ATGAATGGAGTGTTATGGAACGG |
| | (SEQ ID NO. | NO. 108) | ACT (SEQ ID NO. 176) |
| | 107) | | |
| u15pp012 | AAAAAAATGG | ATTCCATTGGA | AAAAAAATGGAATCCAAAATCAT |
| | AATCCAAAAT | GTCAATTCCTTT | TGACTGGAAAGGCTGGGTGTCGA |
| | CATTGACTGG | CGACACC (SEQ | AAGGAATTGACTCCAATGGAAT |
| | (SEQ ID NO. | ID NO. 110) | (SEQ ID NO. 177) |
| | 109) | | |
| u15pp052 | AATGTAATGA | ATTGGAGTCCA | AATGTAATGAACTTTAATGGAATG |
| | ACTTTAATGGA | TTCACTTCCAGA | TACTCGAATGGATTCGACTGGAAT |
| | ATGTACTCG | ACATTCC (SEQ | GGAATGTTCTGGAAGTGAATGGAC |
| | (SEQ ID NO. | ID NO. 112) | TCCAAT (SEQ ID NO. 178) |
| | 111) | | |
| u15pp111 | AATGGAAAGG | TTTCCAGTACAT | AATGGAAAGGAATTGAATGGAGT |
| | AATTGAATGG | TTCATTCCAATC | AGATTGGATTGGATGGGATTGGAA |
| | AGTAGATGGG | CCATCC (SEQ ID | TGAAATGTACTGGAAA (SEQ ID |
| | (SEQ ID NO. | NO. 114) | NO. 179) |
| | 113) | | |
| u15pp084 | AATGGAATGG | ATTCTCTTCTAC | AATGGAATGGAATTGAATGGAAT |
| | AATTGAATGG | TGCATACAATTT | GGGAACGAATGGAGTGAAATTGT |
| | AATGGGAACG | CACTCC (SEQ ID | ATGCAGTAGAAGAGAAT (SEQ ID |
| | (SEQ ID NO. | NO. 116) | NO. 180) |
| | 115) | | |
| u15pp104 | CAATGGAATA | CATTTGATTTGA | CAATGGAATAGAATGGAACGAAA |
| | GAATGGAACG | TTCCATTGATTT | TTTCACGGAATGGAATCAAACTGA |
| | AAATTTCACG | GATTCC (SEQ ID | ATGGAATCAAATCAATGGAATCA |
| | (SEQ ID NO. | NO. 118) | AATCAAATG (SEQ ID NO. 181) |
| | 117) | | |
| u15pp077 | TGGAAAGGAA | ATTACATTCGTG | TGGAAAGGAATGGACTCAAATTG |
| | TGGACTCAAA | TTCATTCCATTC | AAAGGGCTCGAAAGGAATGGAGT |
| | TTGAAAGGGC | CAGACC (SEQ ID | CAAATGGAATGGTCTGGAATGGA |
| | (SEQ ID NO. | NO. 120) | ATGAACACGAATGTAAT (SEQ ID |
| | 119) | | NO. 182) |
| u15pp129 | ATTGGAATGG | ATTCGAGACCG | ATTGGAATGGAAGGGAATGTAGT |
| | AAGGGAATGT | TAGCATTCCACT | GTAATGGACAGGCCTGGAATAAA |
| | AGTGTAATGG | TTATTCC (SEQ | GTGGAATGCTACGGTCTCGAAT |
| | (SEQ ID NO. | ID NO. 122) | (SEQ ID NO. 183) |
| | 121) | | |
| u15pp137 | AATGGAATGC | AGGCCTGTCCA | AATGGAATGCAAGCGAAAGGAAA |
| | AAGCGAAAGG | TTACACTACATT | GGAATGGATTGGAATGGAATGGA |
| | AAAGGAATGG | CCCTTCC (SEQ | ATTCATTGGAATGGAAGGGAATGT |
| | (SEQ ID NO. | ID NO. 124) | AGTGTAATGGACAGGCCT (SEQ ID |
| | 123) | | NO. 184) |
| u15pp131 | ATTGGAATGG | CGAGACCGTAG | ATTGGAATGGAAGGGAATGTAGT |
| | AAGGGAATGT | CATTCCACTTTA | GTAATGGACAGGCCTGGAATAAA |
| | AGTGTAATGG | TTCCAGG (SEQ | GTGGAATGCTACGGTCTCG (SEQ |
| | (SEQ ID NO. | ID NO. 126) | ID NO. 185) |
| | 125) | | |
| u15pp132 | ATTGGAATGG | GAGACCGTAGC | ATTGGAATGGAAGGGAATGTAGT |
| | AAGGGAATGT | ATTCCACTTTAT | GTAATGGACAGGCCTGGAATAAA |
| | AGTGTAATGG | TCCAGGC (SEQ | GTGGAATGCTACGGTCTC (SEQ ID |
| | (SEQ ID NO. | ID NO. 128) | NO. 186) |
| | 127) | | |
| u15pp145 | ATTGGAATGG | AGACCGTAGCA | ATTGGAATGGAAGGGAATGTAGT |
| | AAGGGAATGT | TTCCACTTTATT | GTAATGGACAGGCCTGGAATAAA |
| | AGTGTAATGG | CCAGGCC (SEQ | GTGGAATGCTACGGTCT (SEQ ID |
| | (SEQ ID NO. | ID NO. 130) | NO. 187) |
| | 129) | | |
| u15pp187 | AATGGACAGG | ATTCCATACATT | AATGGACAGGCCTGGAATAAAGT |
| | CCTGGAATAA | TTTATTCCATTC | GGAATGCTACGGTCTCGAATGGAA |
| | AGTGGAATGC | GAGACC (SEQ ID | TAAAAATGTATGGAAT (SEQ ID |
| | (SEQ ID NO. | NO. 132) | NO. 188) |
| | 131) | | |
| u15pp183 | AATGGAATGG | ATTCCATTTCTT | AATGGAATGGTCTGGAATGGAAT |
| | TCTGGAATGG | TATATTCCATGC | GAACACGAATGTAATGCAACCCA |
| | AATGAACACG | CATTCG (SEQ ID | ATAGAATGGAATCGAATGGCATG |
| | (SEQ ID NO. | NO. 134) | GAATATAAAGAAATGGAAT (SEQ |
| | 133) | | ID NO. 189) |
| u15pp207 | AGTGGAATGG | ATTGTATTCGAT | AGTGGAATGGAATCGAATATAAT |
| | AATTGAATAT | CCCATTCCTTTC | GGAGTCGAATGAAATGGAATTGA |
| | AATGGAGTCG | AATTCC (SEQ ID | AAGGAATGGGATCGAATACAAT |
| | (SEQ ID NO. | NO. 136) | (SEQ ID NO. 190) |
| | 135) | | |
| u15pp176 | AATGCAATGG | CCATTCGTTTCG | AATGCAATGGAATCTAATGAAAC |
| | AATCTAATGA | ATTCCTTTCCAT | GGAAAGGAAAGGAATGGAATGGA |
| | AACGGAAAGG | TCCAGC (SEQ ID | ATGGAATGGGCTGGAATGGAAAG |
| | (SEQ ID NO. | NO. 138) | GAATCGAAACGAATGG |
| | 137) | | (SEQ ID NO. 191) |
| u15pp177 | AATGGCATCA | ATTCCAGTCCAT | AATGGCATCAAACGGAATGGAAT |
| | AACGGAATGG | TAGTTTCGACTC | GGACAGCCACGGAATGGAATGCA |
| | AATGGACAGC | CATTGC (SEQ ID | CTCGAATGCAATGGAGTCGAAACT |
| | (SEQ ID NO. | NO. 140) | AATGGACTGGAAT (SEQ ID NO. |
| | 139) | | 192) |

Amplicon Seq02 from Table 5 was aligned against the corresponding human genome reference sequence using UCSC public browser software (https://genome.ucsc.edu). Visualized is a Y chromosome region relevant to the aligned sequence chosen, Seq02, which is on the q arm of the Y chromosome. This visualization indicates the Seq02 amplicon sequence occurs at least 16 times on the Y chromosome. It is important to note that these regions were identified bioinformatically when selecting for regions that are repeated multiple times but also are unique to the Y chromosome. Unexpectedly, experimental data obtained in the following Examples showed that this region is much more highly repeated than the alignment to the reference sequence suggests. In part this could be caused by the fact that repeat regions are very hard to map and hence the accuracy of the reference genome is low in these regions.

### Example 5. Realtime Assay from Regions with Multiple Copies of Target Sequences

In order to determine if amplification of a highly repetitive Y-chromosome region (HRYR) translated into greater amplification efficiency, especially as it pertains to circulating cell-free DNA (ccfDNA), several amplicons from the HRYR using quantitative real-time PCR were examined. Genome equivalents (GE) were used to describe the amount of material being amplified per loci. Single copy sex-determining region Y gene (SRY) was used to define genome equivalents for comparisons between samples and assays. An assay from the Y-chromosome specific repetitive DNA family (DYZ1) region was also incorporated of the Y chromosome as a reference. The amplification efficiency of this region was determined to be 50-100 times greater than that of SRY (e.g., 90 GEs of DYZ1 were generated for each 1 GE of SRY). FIG. 11 shows the findings from several HRYR loci based on a serial titration of GEs (100-1) with male ccfDNA. As shown in FIG. 11, the four loci all show a clear decrease in GEs with decreasing input as expected for a true quantitative measurement as does DYZ1. Additionally, all four assays from the HRYR show a 30-40-fold increase in GEs amplified relative to DYZ1 which equates to a greater than 1500-fold increase relative to SRY.

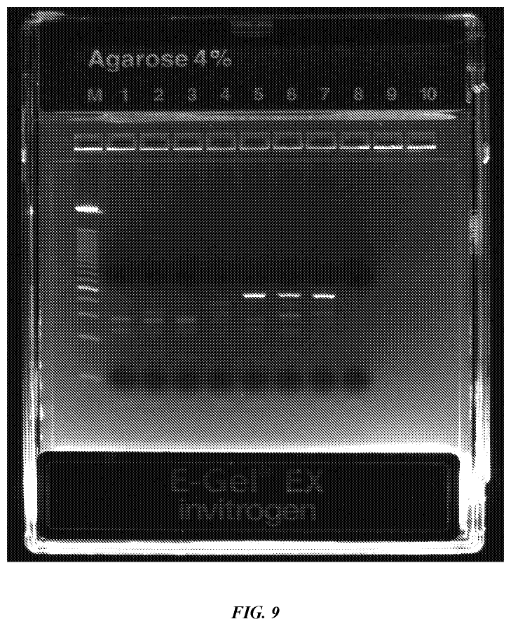

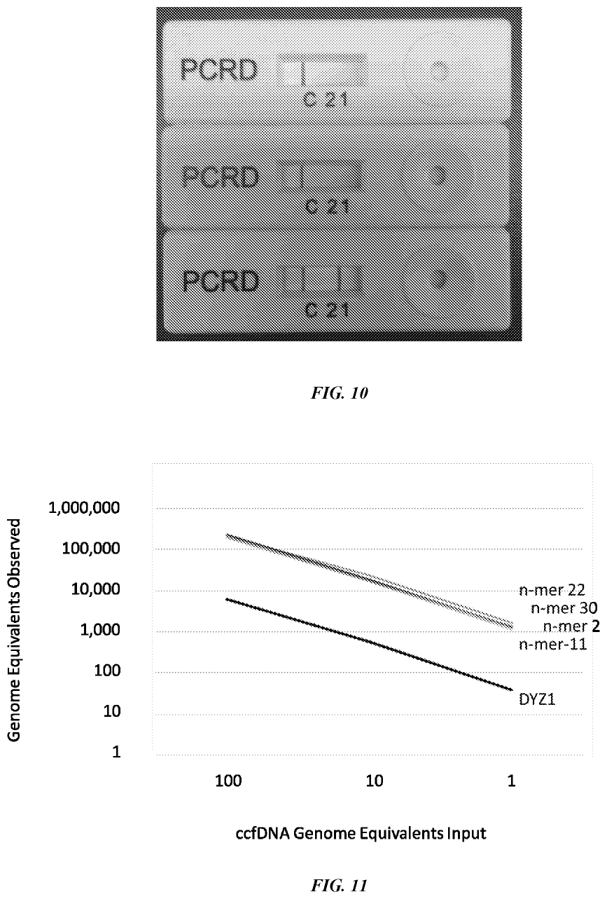

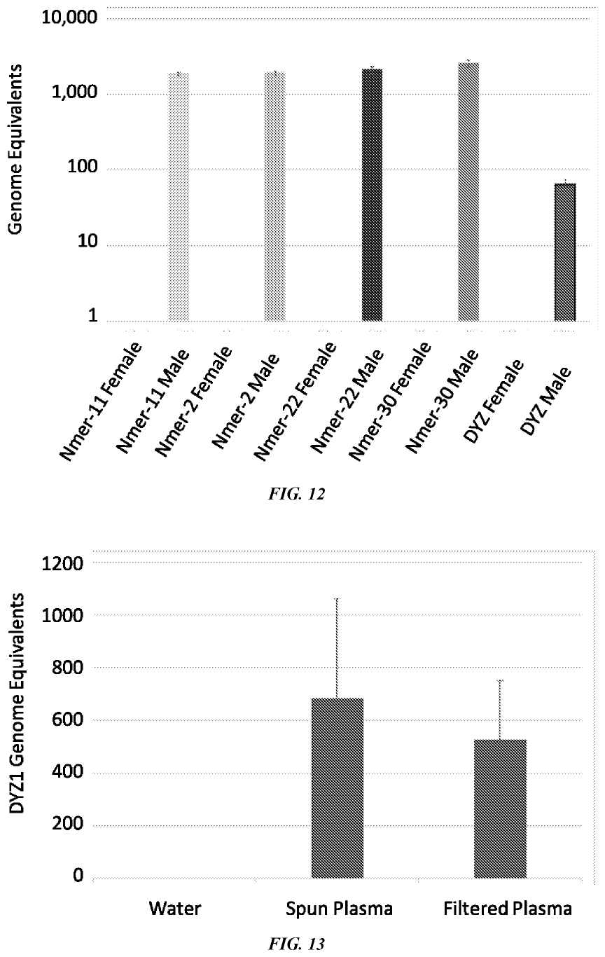

The specificities of the HRYR loci were also tested by comparing the GEs generated from male or female ccfDNA samples in order to establish that the HRYR is truly unique to the Y-chromosome. As seen in FIG. 12, no GEs were generated for the female samples, whereas the male samples generated between 1800-2600 GEs as expected using 1 SRY GE equivalent as a template.

### Example 6. Region Independent Data from Less than 50 μl of Blood, Separated with a Pall Vivid™ Membrane

One important functionality of an exemplary device is the separation of plasma from whole blood in order to minimize host DNA (or in case of prenatal testing, maternal DNA) background. This separation improves sensitivity and specificity. For non-invasive determination of the fetal gender early in pregnancy, the device specifically detects fetal Y-chromosome copies from ccfDNA. Multiple membranes were tested for their ability to filter the plasma component from human whole blood. The most effective was found to be the Pall Corporation Vivid™ Plasma Separation Membrane (hereinafter the Vivid™ Membrane). FIG. 13 shows a comparison between plasma separated from less than 50 microliters (μ1) of male whole blood using the Vivid™ Membrane vs. the standard centrifugation methodology standardly used for the measurement of ccfDNA biomarkers. Viability of the plasma was measured based on the amount of Y-chromosomal ccfDNA as assayed by DYZ1 via qPCR. Nucleic acid (ccfDNA) was isolated and purified from 10 μl of plasma using a paramagnetic bead-based methodology modified for use (MagMax™ kit from Ambion/Thermo Fisher downscaled to use with a 10 μl sample) with such low volumes of plasma. As shown in FIG. 13, both the spun plasma and Vivid™ Membrane separated plasma yielded 500 or greater GEs of DYZ1 as normalized to 1 GE of SRY. The mean copies yielded from the two methods showed no difference based on paired t-test of the two methods (p-value 0.087, accept null hypothesis).

This example shows that plasma can be generated with a filtration step. The amount of blood used in the experiment is small (50 μl), so this is proof that small amounts of plasma can be obtained efficiently from low input amounts of blood without centrifugation. This is important because a centrifuge cannot be used as a method in point of care (also referred to as point of need) devices.

### Example 7. Data from DNA Extraction with Low Amounts of Plasma (10 μl to 40 μl): Bead-Based Versus Column-Based

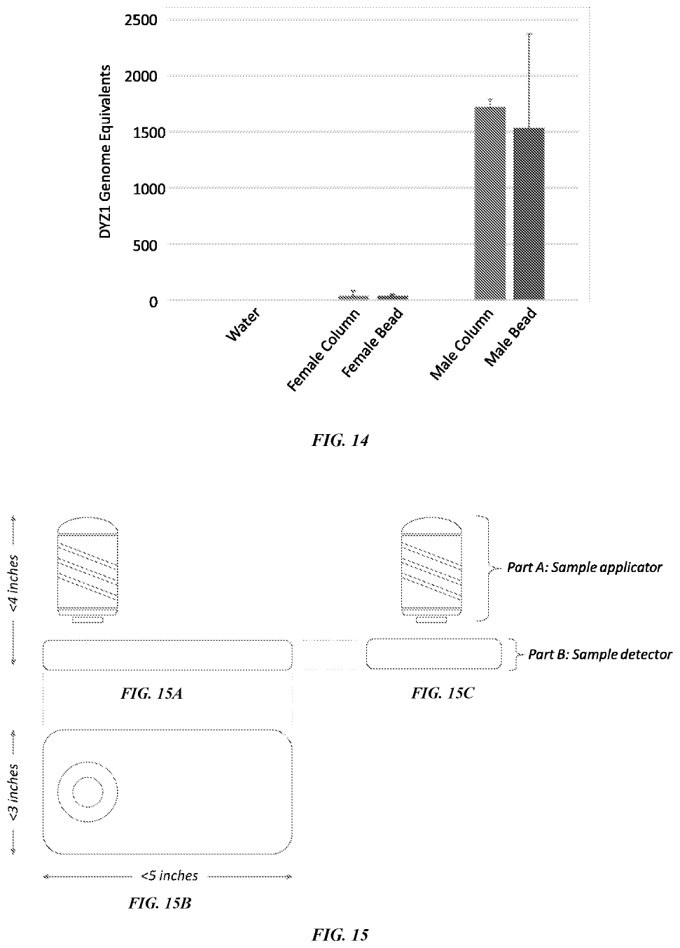

Robust isolation and purification of ccfDNA from low volumes of plasma (10 μl to 40 μl) is an important characteristic of the fetal sex determination device. Two primary methods of nucleic acid isolation were modified and tested to accommodate such low volumes, column-based and paramagnetic bead-based. Commercial versions of these methods were employed for proof-of-principal purposes. Column-based extractions were performed with the Qiagen Investigator Kit and bead-based extractions using the ThermoFisher MagMax Kit. Efficacy of the methods to extract amplifiable ccfDNA was measured using qPCR with primers for DYZ1. FIG. 14 shows the comparative yields from the two methods with 20 μl of human plasma as input for extraction from male and female subjects. As seen both methods yielded greater than 1500 GEs as measured by DYZ1 normalized to 1 GE of SRY. No difference was observed between the two methods based on a paired t-test (p-value 0.732, accept null hypothesis). Extraction of water or female plasma samples yielded negligible amounts of amplifiable target. The data demonstrate that extraction of amplifiable ccfDNA from very low plasma volumes (10 μl to 20 μl), such as those expected from a 20 μl to 40 μl blood sample, is achievable. FIG. 13 also demonstrates the viability of ccfDNA extraction from 10 ul of plasma following plasma membrane separation of 40 μl of blood.

While preferred embodiments of the present methods, devices, systems and kits disclosed herein have been shown and described herein, it will be obvious to those skilled in the art that such embodiments are provided by way of example only. Numerous variations, changes, and substitutions will now occur to those skilled in the art without departing from the methods, devices, systems and kits disclosed herein. It should be understood that various alternatives to the embodiments of the methods, devices, systems and kits described herein may be employed in practicing the methods of using the devices, systems and kits disclosed herein. It is intended that the methods and structures within the scope of these claims and their equivalents be covered thereby.

What is claimed is:

1) A device comprising:
a) a sample purifier for removing a cell from a biological fluid sample to produce a cell-depleted sample; and
b) at least one of a detection reagent and a signal detector for detecting a plurality of cell-free DNA fragments in the cell-depleted sample.

2) The device of claim 1, wherein a first sequence is present on a first cell-free DNA fragment of the plurality of cell-free DNA fragments and a second sequence is present on a second cell-free DNA fragment of the plurality of cell-free DNA fragments, and wherein the first sequence is at least 80% identical to the second sequence.

3) The device of claim 2, wherein the device comprises at least one nucleic acid amplification reagent and a single pair of primers capable of amplifying the first sequence and the second sequence.

4) The device of claim 2, wherein at least one of the first sequence and the second sequence is repeated at least twice in a genome of a subject.

5) The device of claim 2, wherein the first sequence and the second sequence are each at least 10 nucleotides in length.

6) The device of claim 2, wherein the first sequence is on a first chromosome and the second sequence is on a second chromosome.

7) The device of claim 2, wherein the first sequence and the second sequence are on the same chromosome but separated by at least 1 nucleotide.

8) The device of claim 2, wherein the first sequence and the second sequence are in functional linkage.

9) The device of claim 1, wherein the sample purifier comprises a filter, and wherein the filter has a pore size of about 0.05 microns to about 2 microns.

10) The device of claim 9, wherein the filter is a vertical filter.

11) The device of claim 1, wherein the sample purifier comprises a binding moiety selected from an antibody, antigen binding antibody fragment, a ligand, a receptor, a peptide, a small molecule, and a combination thereof.

12) The device of claim 11, wherein the binding moiety is capable of binding an extracellular vesicle.

13) The device of claim 2, wherein the at least one nucleic acid amplification reagent comprises an isothermal amplification reagent.

14) The device of claim 1, wherein the signal detector is a lateral flow strip.

15) The device of claim 1, wherein the device is contained in a single housing.

16) The device of claim 1, wherein the device operates at room temperature.

17) The device of claim 1, wherein the device is capable of detecting the plurality of biomarkers in the cell-depleted sample within about five minutes to about twenty minutes of receiving the biological fluid.

18) The device of claim 1, comprising a communication connection.

19) The device of claim 1, comprising a transdermal puncture device.

20) A method comprising:
a) obtaining a fluid sample from a subject, wherein the volume of the biological sample is not greater than about 120 microliters;
b) contacting at least one cell free nucleic acid in the fluid sample with an amplification reagent and an oligonucleotide primer that anneals to a sequence corresponding to a sequence of interest in order to produce an amplification product; and
c) detecting the presence or absence of the amplification product, wherein the presence or absence indicates a health status of the subject.

21) The method of claim 20, wherein the fluid sample is a blood sample.

22) The method of claim 20, wherein the fluid sample is a plasma sample from blood.

23) The method of claim 22, wherein the volume of the plasma sample is not greater than 50 μl.

24) The method of claim 22, wherein the volume of the plasma sample is between about 10 μl and about 40 μl.

25) The method of claim 20, wherein the sample contains about 25 pg to about 250 pg of total circulating cell free DNA.

26) The method of claim 20, wherein the sample contains about 5 to about 100 copies of the sequence of interest.

27) The method of claim 26, wherein the copies are at least 90% identical to one another.

28) The method of claim 20, wherein the sequence of interest is at least 10 nucleotides in length.

29) The method of claim 20, wherein contacting comprises performing isothermal amplification.

30) The method of claim 20, wherein contacting occurs at room temperature.

31) The method of claim 20, wherein the method comprises incorporating a tag into the amplification product as the amplifying occurs, and wherein detecting the presence of the amplification product comprises detecting the tag.

32) The method of claim 31, wherein the tag does not comprise a nucleotide.

33) The method of claim 31, wherein detecting the amplification product comprises contacting the amplification product with a binding moiety that is capable of interacting with the tag.

34) The method of claim 33, comprising contacting the amplification product with the binding moiety on a lateral flow device.

35) The method of claim 20, wherein the steps (a) through (c) are performed in less than fifteen minutes.

36) The method of claim 20, wherein the method is performed by the subject.

37) The method of claim 20, wherein the method is performed by an individual without receiving technical training for performing the method.

38) The method of claim 20, wherein obtaining, contacting, and detecting is performed with a single handheld device.

39) The method of claim 20, wherein the health status is selected from the presence and the absence of a pregnancy.

40) The method of claim 20, wherein the health status is selected from the presence and the absence of a neurological disorder, a metabolic disorder, a cancer, an autoimmune disorder, an allergic reaction, and an infection.

41) The method of claim 20, wherein the health status is a response to a drug or a therapy.

42) A device comprising:
a) a sample purifier that removes a cell from a fluid sample of a female subject;
b) at least one nucleic acid amplification reagent;
c) at least one oligonucleotide comprising a sequence corresponding to a Y chromosome, wherein the at least one oligonucleotide and nucleic acid amplification reagent are capable of producing an amplification product; and
d) at least one of a detection reagent or a signal detector for detecting the amplification product.

43) The device of claim 11, wherein the oligonucleotide comprises a sequence corresponding to a gene selected from DYS14 gene or a TTTY22.

44) A method comprising:
a) obtaining a fluid sample from a female pregnant subject, wherein the volume of the biological sample is not greater than about 300 microliters;
b) contacting at least one cell free nucleic acid in the fluid sample with an amplification reagent and an oligonucleotide primer that anneals to a sequence corresponding to a sex chromosome; and
c) detecting the presence or absence of an amplification product, wherein the presence or absence indicates the gender of a fetus of the female pregnant subject.

45) The method of claim 43, wherein the fluid sample is a blood sample.

46) The method of claim 44, wherein the volume of the blood sample is not greater than 120 μl.

47) The method of claim 43, wherein the fluid sample is a plasma sample from blood.

48) The method of claim 46, wherein the volume of the plasma sample is not greater than 50 μl.

49) The method of claim 46, wherein the volume of the plasma sample is between about 10 μl and about 40 μl.

50) The method of any one of claims 43 to 48, wherein obtaining comprises performing a finger prick.
